(19) United States
(12) Patent Application Publication
(10) Pub. No.: US 2026/0204387 A1
(43) Pub. Date: Jul. 16, 2026

(54) SYSTEM AND METHOD FOR ARTIFICIAL INTELLIGENCE ANALYZATION AND SCORING OF CONSUMABLES

(71) Applicant: IndulgeRx Brands Inc., Woods Cross, UT (US)

(72) Inventors: John L. Hastings, III, North Salt Lake, UT (US); Sarah E. Hastings, North Salt Lake, UT (US); John A. C. Hastings, West Bountiful, UT (US); Ernest R. Dragoo, Eden, UT (US)

(73) Assignee: IndulgeRx Brands Inc., Woods Cross, UT (US)

(21) Appl. No.: 19/355,278

(22) Filed: Oct. 10, 2025

Related U.S. Application Data
(60) Provisional application No. 63/745,875, filed on Jan. 16, 2025.

(51) Int. Cl. G16H 20/60; G16H 10/60; G16H 15/00

(52) U.S. Cl. CPC G16H 20/60; G16H 10/60; G16H 15/00; G16H 50/30; G16H 50/70

(57) ABSTRACT

The present application is directed to a system which integrates advanced scientific methods, and artificial intelligence (AI) enabled software, applications and algorithms to assess and optimize health impacts of existing consumables and theoretical consumable formulations, artificial and natural ingredients, and associated bio-functional effects, for purposes of generating actionable Relevant Nutrition Scores (RNS's). RNS AI-enabled software, algorithms and applications, systems & methods analyze comprehensive food information, food preparation practices, additives, preservatives and packaging by weighting user-aligned inputs. RNS supports global product assessments inclusive of health benefits, opportunities and risks, dietary choices, food innovations, purposeful health certifications, and healing and recovery protocols, achieved through dynamic, multi-dimensional nutritional research and applied health impacts, each and all aligned in support of specific diverse users. System outputs optimize existing, speculative and future consumable formulations, deliberate dietary recommendations, purposeful products production, treatment protocols, and pharmaceutical efficacies, while promoting wellness, disease resilience and natural immunities.

### FIELD OF THE INVENTION

The present invention is directed to a proprietary system⁽¹⁾ which integrates advanced scientific methods⁽²⁾, and artificial intelligence (AI) enabled software, applications⁽³⁾ and algorithms⁽⁴⁾ to assess and optimize health impacts⁽⁵⁾ of existing consumables and theoretical consumable formulations⁽⁶⁾, artificial and natural ingredients⁽⁷⁾, and associated bio-functional effects⁽⁸⁾, for purposes of generating actionable Relevant Nutrition Scores (RNS's)⁽⁹⁾. More particularly, RNS AI-enabled software, algorithms and applications⁽¹⁰⁾, systems & methods analyze comprehensive food informationd⁽¹¹⁾, food preparation practices⁽¹²⁾, additives⁽¹³⁾, preservatives⁽¹⁴⁾ and packaging⁽¹⁵⁾ by weighting user-aligned inputs⁽¹⁶⁾. RNS supports global product assessments⁽¹⁷⁾ inclusive of health benefits, opportunities and risks, dietary choices⁽¹⁸⁾, food innovations⁽¹⁹⁾, purposeful health certifications, and healing and recovery protocols⁽²⁰⁾, achieved through dynamic, multi-dimensional nutritional research and applied health impacts, each and all aligned in support of specific diverse users. System outputs optimize existing, speculative and future consumable formulations, deliberate dietary recommendations, purposeful products production, treatment protocols, and pharmaceutical efficacies⁽²¹⁾, while promoting wellness, disease resilience and natural immunities⁽²²⁾. For references numbered herein, please see the Glossary of Terms beginning on page 14 of this specification.

### BACKGROUND OF THE INVENTION

Many people struggle to make informed, intentional or purposeful consumable, food and nutrition choices given the many misunderstandings, misapplications and misappropriations of simple nutrition or ingredient panel terms and concepts like calories, fats, carbohydrates, sugars, sodium, proteins, artificial or natural ingredients, or other. In the broad marketplace, people do not truly have transparent lines of sight to anything about good or bad sources or qualities, or any short or long-term health impacts related to the benefits or risks of product sourcing, producing, processing, preserving, packaging, or preparing. And even so-called regulatory “standards” are often incomplete or insufficient to protect consumers, given large loopholes and known workarounds, domestically and globally.

And unfortunately, neither consumers nor consumer package goods (CPG) manufacturers truly appreciate the realized or comprehensive health impacts of final products, once consumed. Health objectives or appreciated consequences of eating foods from “fresh with pesticides” to “packaged with preservatives”, are simply only as good as the product or packaging disclosures made for any foods consumed, especially given largely incomplete, inconsistent or intentionally misrepresented health claims, so-called benefits or claimed qualities. There is no valid standard.

Understanding how casual or causal consumption of foods or needed nutrition is complex and not easily understood, especially as it relates to how ingredients or nutrition will functionally interact with and impact a person or relevant user. And beyond general scoring, grading or so-called health tracking applications for targeted general populations or afflicted subsets of at-large populations (for any age, life stage or lifestyle, culture, environment or health status), transparency of understanding of the real health risks and benefits is a complex inter-related challenge for consumers and CPG businesses alike, as well as for medical providers and governing bodies. This lack of vantage-point applied or relevant actionable understanding is due to the highly individualized and functional nature of dietary needs and drivers, and the myriad of related and disparate factors influencing both health and resulting nutritional impacts throughout entire food supply chains from “soil to stomach”.

For consumers, the difficulty lies in easily identifying the relevant functional nutrients in foods that actually support consumer-specific health conditions or objectives, lifestyle demands, and genetic or chronic predispositions. These health drivers differ widely even among like individuals of the same age or health status, opt-in or imposed dietary preferences, and the many influential external factors of life and stresses beyond dietary habits. Businesses, particularly in food, and health & wellness industries, face ever-mounting challenges in developing products and services that are broadly effective, yet relevant and customizable to individual health and nutrition needs, always balancing convenience and price vs. accessibility and quality. Governing bodies also face ever-changing impacts of scientific research, environmental contaminants, deficient dietary understandings, production, preservation and packaging practices, combined with ever-evolving medical studies, often updating or even reversing prior health understandings. As a result, manufacturing processes, supply chains, dietary drivers, nutritional understandings, and biological implications, as well as health legislation and applications are often mass-customized, incomplete, erroneous and outdated, including but not limited to: USA: US FDA/USDA health claims' guidelines, nutrition panels, GRAS or “Generally Recognized as Safe” standards, and Food Pyramids, etc.; European: (EC) No 1924/2006 on nutrition and health claims; (EU) No 1169/2011 on food information to consumers (nutrition labeling); European Food Safety Authority (EFSA) scientific evaluation of health claims; EU Register of nutrition and health claims; Asian: China, GB 28050-2011 standard for nutrition labeling; Chinese Health food registration and filing system for functional claims; Chinese Food Guide Pagoda; Japanese Foods with Health Claims system; Japanese Foods with Nutrient Function Claims (FNFC); Japanese Foods for Specified Health Uses (FOSHU); Japanese Foods Function Claims (FFC); Japanese Food Guide Spinning Top; Southeast Asia, ASEAN Common Principles for Health Claims; Country-specific regulations (e.g. Malaysia, Singapore, Thailand); Southeast Asia, Food-based dietary guidelines & regional food guides; Arab Countries, Gulf Cooperation Council (GCC) standards on nutrition labeling and health claims; Arab Food Dome dietary guidelines; Country-specific food guides (e.g. Lebanese Cedar Food Guide, Saudi Food Palm), etc.

IndulgeRx Brands Inc. (“IRX”), Relevant Nutrition Score (“RNS” and “RNS AI”), enabling the integrated methods, systems, software applications and services known as IRX FOODSCORE, a dynamic AI-enabled ecosystem including the aforementioned RNS, as well as relevant Consumable or Food Health Risk Profiling & Warnings, and Consumable or Food Health & Claims Compliance. These complexities are further compounded and exacerbated by need for continuous adaptations of dietary reactions and recommendations related to short-term or protracted health issues, and to situational or environmental impacts, subject to evolution of biological and scientific understandings, and as individuals progress through different life stages and health scenarios. The failures of “one-size-fits-all” solutions necessitate advanced tools and technologies, such as IRX Artificial Intelligence (AI), IRX Machine-Learning (ML) and IRX Deep learning (DL), as well as IRX Cloud Services including but not limited to SaaS (IRX Software as a Service), PaaS (IRX Platform as a Service), DaaS (IRX Device as a Service), IaaS (IRX Infrastructure as a Service), DaaS (IRX Desktop as a Service), and BaaS (IRX Back-end as a Service) to analyze, interpret, integrate, and score user-specific relevance of the vast disparate amounts of data related to personal, target groups and societal dietary health, making relevant nutrition a promising & challenging frontier in consumables, foods, medications and healthcare.

The IRX FOODSCORE invention revolutionizes the way people perceive and assess real benefits, relevant risks, and engage to consume foods, intentional nutrition, dietary choices and pharmaceuticals, objective and situational. This invention integrates cutting-edge AI-enabled technologies, bio-scientific advancements and dynamic data-driven analytics to address the acute challenges of evaluating and understanding purposefully functional nutritional values, individual health impacts and personal relevancy, short and long-term. By providing a comprehensive user-friendly solution, IRX FOODSCORE fundamentally reshapes the landscape of nutritional understandings and applied biosciences, enabling individuals to make informed, purposeful dietary and real health-risk decisions tailored to unique health goals and personal preferences.

The IRX FOODSCORE invention represents a holistic, significant advancement in integrating and applying persistent, harmonized data related to diverse global populations, cultures, diets, foods, nutrition, and situational environments and climates, aligned with respective health practices, and considering all available bioscientific data and research, while applying all available medical, health and wellness learnings, and combined with big data analytics in furtherance of simplified resolutions to real-world problems or opportunities related to food safety, nutrition, health and environmental sustainability.

The IRX FOODSCORE outputs are tailored to individual and targeted community needs globally, and relevant health & nutrition scoring & grading, health & risk profiling, and health & claims compliance of a food, beverage or medication's purposeful impacts on general or specific health, wellness and situational lifestyles, active or impaired, and across any culturally aligned age (pre-birth to death), lifestyle (situational or opt-in) or life-stage (active or sedentary). And the IRX FOODSCORE is uniquely grounded in the studied bioavailability and immune-response interactions (positive, neutral or negative) with any known disease-treatment or health impact protocols (western or eastern), pharmaceutical or over-the-counter products, supplements or illegal drugs, and their respective side-effects or interactions.

The IRX FOODSCORE invention leverages advanced AI tools to ensure that purposeful nutritional values and applied bio-sciences are reliable and consistent across different countries and cultures, which is a material problem for addressing compliant nutrition standards in support of any health objective or in response to any disease state. This is achieved by an IRX FOODSCORE as it allows for endless non-denominational diet and nutritional application platforms with related risks or benefits, which enable applied nutritional bio-sciences and learnings within a localized framework of unbiased applications in support of international organizations, national governments, food industry stakeholders, and scientific communities. IRX FOODSCORE becomes a universal currency of an applied bio-scientific nutritional exchange, where no other exists today.

There have been global standardization and harmonization challenges and failures across international organizations like the Food and Agriculture Organization (FAO) and the World Health Organization (WHO), both of which play crucial roles in developing global general directional standards (passive vs. applied) and macro guidelines for food composition data. However, the International Network of Food Data Systems (INFOODS) is a global initiative by the FAO aimed at improving food composition data quality and availability worldwide, a framework that would greatly benefit from a nutrition opportunity and risk assessment ecosystem such as IRX FOODSCORE, as it normalizes objectives and exposes applied supply chain issues.

The above referenced organizations (and many others globally) will be potential sources of raw data inputs (as a partial input, not as an end result) in support of IRX FOODSCORE. However, the organizations above will benefit more from never-before available applied nutritional currencies of exchange that are dynamically enabled by IRX FOODSCORE which are created and facilitated proactively or reactively for a single country or across countries. Unlike any other platform, application, software or service, the IRX FOODSCORE platform will support the integrated adoption and implementation of true first-of-their-kind international standards, in furtherance of reliable consistency, relevant applicability and functional impacts of nutritional values (at-work) within and across different cultures and countries, and at any age or stage of life, and regardless of health, disease, activity, biological or situational influencers.

In general, some minimal efforts have been made to harmonize national food composition databases through international collaboration, but these are heavily dependent upon global governance, funding and cooperation, and all politics aside. And yet despite slow progress, some projects like European Food Information Resource (Euro FIR) aim to standardize and harmonize food composition data in Europe. Similar initiatives are slowly adopted in other regions to ensure that food composition data are comparable across countries, wherein the IRX FOODSCORE “neutrally” fills this gap of globally applied, purposeful nutrition and food supply chain product sourcing & procurement, production, preservation, packaging and preparation practices.

Strengthening National Regulatory Frameworks: Countries have tried to establish and enforce strict regulatory frameworks for directionally accurate and informative food labeling and nutritional information. (e.g., FDA, USDA and EFSA, etc.). This includes setting mandatory requirements for the declaration of certain macro nutrients, calories, proteins, fats, sugars, salts, and some limited vitamins and minerals, and others, while adopting standardized methods for any localized or globalized nutrient analyses.

Regular Monitoring and Verification: Global and national food safety and standards authorities episodically, infrequently and arbitrarily monitor and verify nutritional information provided by domestic or international food manufacturers, sometimes involving one-off random testing on food products to ensure that the nutritional values listed on labels match those determined through laboratory analyses. However, this does not translate into any meaningful actions or applied reliability on behalf of consumers. The current information is directional at best, whereas the IRX FOODSCORE provides immediate, transparent nutritional understanding of applicability and purposeful benefits or associated risks of foods to be consumed.

Some governments, random research institutions and private health and diet Apps are investing in scientific research to improve the accuracy and comprehensiveness of reported food composition data by flawed historical standards, not applied. This includes studying the effects and impacts of processing, storage, preservation and preparation methods on the nutritional content of foods. Conversely, the IRX FOODSCORE considers all of this within an integrated (not segregated) framework for defining a new multi-dimensional, applied nutrition standard.

One of the great obstacles to addressing purposeful uses and nutritional impacts has been the failed development of advanced analytical methods. The advancement of AI-enabled analytical technologies and methods is crucial for accurately determining the relevant nutritional content of foods and applied uses of products, but as of today they lack functional frameworks for objective or purposeful uses in their current forms. Laboratories have yet to adopt a standardized, validated analytical method for nutrient impacts, optimization, interactions, metabolization and purposeful use analyses, as recommended by several international organizations like AOAC International. And, while this is deemed somewhat progressive, it falls far short of the IRX FOODSCORE ecosystem that provides defined and relevant functional benefits (e.g., relevant nutrition scores, risk profiling, claims compliance, etc.) inherently within a uniquely single and simple score of products for any target population or for defined end-users.

One of the great challenges today is that there are broadscale industry compliance and consumer education shortfalls. From an industry compliance perspective, food, beverage and medication manufacturers comply with simple national and international standards for use, labeling, nutritional and side-effect information, which include the reporting of so-called nutritional or use content for products and labeling updates due to product formulation changes.

From an education perspective, consumer reading and understanding of product use or nutritional labels is opportunistic, when accurate or applicable, however, current disclosure and transparency efforts fall far short of the stated objectives, whereas the IRX FOODSCORE is founded upon the collective and integrated cornerstones of all things that contribute to a net new, applied health transparency and applicability standards (dynamically), which are immediately relevant for any end-user or population.

IRX is a pioneer in purposeful nutrition while overcoming the persistent problem of available, accurate and consistent data. IRX simply approaches the problem differently through the creation of a first-of-its-kind, AI-enabled Nutritional Refinery that systematically processes many diverse sources of external influential, nutritional, situational, environmental, biological and medical research data and combine it like never before. This proprietary data refinery is deployed to systematically produce a dynamic and persistent portfolio of diverse, value-added functional fuels necessary to drive our IRX FOODSCORE Solution.

Our groundbreaking IRX FOODSCORE innovation revolutionizes the way people perceive, assess and engage to consume foods, beverages and medications (consumables), intentional nutrition and dietary choices, objective and situational. This approach integrates cutting-edge AI-enabled technologies, bio-scientific advancements and dynamic data-driven analytics to address the acute challenges of evaluating and understanding purposefully functional nutritional values, and individual health impacts, with pronounced personal relevancy, short and long-term.

By providing a comprehensive and user-friendly solution, our IRX FOODSCORE reshapes the landscape of nutritional understandings and food sciences, enabling individuals to make informed, purposeful dietary decisions tailored to unique health goals and personal preferences. Critically, IRX solves the problems defined above and herein for end-users.

Simply stated, IRX takes many disparate sources of product uses, medical, scientific, population, environmental, nutritional, dietary, and health & wellness data, and combine them into: Our revolutionary AI-enabled IRX FOODSCORE refinery, which addresses the complex challenges of navigating intricate consumable product uses and nutritional landscapes, offers a dynamic and analytical understanding of each food's potential, purpose and impact on health; A seamless integration of diverse datasets from global sources, including governmental, institutional, academic, and health & wellness data, whereby our platform creates a user-friendly “nutritional currency” and exchange matrix by applying advanced filtering mechanisms and scoring algorithms; A comprehensive and purposeful nutrition approach that empowers individuals and organizations alike to easily assess macro, micro, and phytonutrient benefits, facilitating informed dietary choices aligned with unique health goals and objectives across diverse cultural, lifestyle, and health states. IRX delivers comprehensive consumable impacts through: relevant health & nutrition scores (RNS) or grades; food health risk profiling & warnings; and via relevant food health and consumable claims compliance scores or grades.

Our revolutionary AI-enabled IRX FOODSCORE refinery systematically addresses the many complex consumer and business challenges of navigating intricate and multi-dimensional data and nutritional landscapes, due to unreliable, ever-evolving and inconsistent nutritional values and so-called food or consumable facts in an environment of infinite and often-conflicting dietary guidelines, options or opinions. This is accomplished while we dynamically and analytically foster a holistic understanding of each unique or combined food's actual purpose, relevancy and functional impact on individual or group health and well-being (at the time of consumption), and whether actively, passively, proactively, preventatively, retroactively and/or responsively utilized in support of a specific heath objective or situation.

Our innovative systematic IRX FOODSCORE refinery seamlessly integrates and navigates the intersections of complexly diverse, disparate and expansive consumable or food, nutritional, dietary, health and disease datasets. This holistic approach encompasses institutional, governmental, academic, bio-scientific, food & pharma, and health & wellness data and analytics sources globally, public and private, and published, studied or observed data, each and all made available to us persistently or episodically through public or private sources, facilitated through downloads, uploads, cloud-based API's, either by free access, license, subscription or purchase.

To address the complex challenges of navigating intricate and multi-dimensional health and nutritional landscapes, various systems and methodologies have been developed to assess, score, and grade the nutritional values and impacts of consumables or foods. These systems aim to provide consumers, businesses, medical providers and governing agencies with reliable, consistent, and holistic nutritional information, despite the ever-evolving and often conflicting dietary metrics, guidelines, and opinions. Here are the most relevant efforts found to date:

Nutri-Score: Developed in France, Nutri-Score is a front-of-pack labeling system that simplifies the nutritional profile of food products into a five-color, letter-coded scale from A (healthiest) to E (least healthy). This system considers both positive and negative nutritional elements, such as fiber, protein, fruits, and vegetables versus sugar, saturated fat, and sodium content. Please see https://www.santepubliquefrance.fr/en/nutri-score.

NuVal: NuVal assesses and rates food on a scale from 1 to 100 based on more than 30 nutrients, with higher ratings indicating better nutritional quality (in general). This system is used in some U.S. supermarkets to help consumers make healthier choices. Please see http://healthscienceandlaw.ca/wp-content/uploads/2016/06/Label.Bernier.2016.pdf.

MyFitnessPal: Core functionality revolves around an extensive food database, which contains over 14 million foods, allowing users to log their food intake either manually or by scanning barcodes. Additionally, Meal Scan technology enables users to log meals by simply pointing their camera at ingredients and recipes. This feature, along with the ability to track macronutrient content and total intake of carbohydrates, fats & proteins, provides users with detailed understanding of dietary habits. Please see https://www.myfitnesspal.com/en/.

FOODUCATE: A comprehensive nutrition & health App designed to help users make healthier food choices and improve their dietary habits. Launched in 2010, Fooducate offers a variety of features aimed at educating users about nutritional content and health implications of the foods they consume. Food Grading System: Fooducate uses an algorithm to assign a grade from A to D to foods based on their nutritional value, with higher grades indicating healthier choices. This grading system considers factors such as the naturalness, healthfulness, and level of processing of the food. Food Database: The App includes a large database of foods, allowing users to track their daily food intake and monitor the quality of their calories. It also provides suggestions for healthier alternatives to less nutritious foods. Please see https://www.fooducate.com/.

MyNetDiary: Is a food logging and health tracking App. It provides a letter grade (A, B, C or D) used by MyNetDiary to rank the general healthfulness of a product. Please see https://www.mynetdiary.com/.

Food Compass: Developed by researchers at Tufts University, this system rates foods on a scale from 1 to 100 based upon a comprehensive algorithm that includes 54 attributes across nine domains, such as nutrient ratios, food-based ingredients, and processing characteristics (directional health, but again generalized for mass audience). This system aims to guide healthier consumer choices and inform policy and industry reformulations. Please see https://sites.tufts.edu/foodcompass/.

FoodScore: Japan-based consumer App-AI graded foods based solely upon the presence of harmful additives, with consumer applied tasting notes. It's passive in that it looks for known effects (study of 7,000 people) and observed additives, then applies grade. Claims “Patent Pending” and Nobel Peace Prize worthiness. Please see https://food-score.tech/lp. Yuka: Food/Cosmetic Evaluation App for mobile devices. Analyzes ingredients (60%), Additives (30%), Organics (10%). It evaluates product's impact on general health. Please see https://yuka.io/en/.

EWG: Environmental application is a simple and easy-to-use database that helps you make more informed choices about what you eat and drink based on scientific data and research. Food and beverages are ranked on their nutrition, ingredient and processing concerns. Each product gets a rating on a scale of 1 to 10—best to worst. Details are also available for each product's breakdown, highlighting concerns and known safety hazards that affect the rating. Please see https://www.ewg.org/foodscores/.

Calorie Mama AI: A calorie counter App that uses AI and machine learning algorithms to recognize various foods, including complex dishes and packaged goods, from user-submitted photos. Please see https://www.caloriemama.ai/.

Life Cycle Assessment (LCA): LCA is used to evaluate environmental impacts of foods, which can be integrated with health assessments to provide a more comprehensive view of food sustainability and health impacts. This method assesses environmental, social & health effects of food products throughout their lifecycle. Please see https://greenly.earth/en-us/.

OECD Holistic Food Systems Approach: This approach emphasizes the synergies and trade-offs between various food system outcomes, such as nutrition, livelihoods, and environmental sustainability. It advocates for policy coherence across different dimensions to enhance the effectiveness of food systems. Please see https://www.oecd.org/food-systems/.

HOLiFOOD Project: Funded by the European Commission, this project aims to tackle food system risks by integrating Big Data and AI technologies to develop early warning systems for food safety hazards. It also focuses on improving data and knowledge-sharing infrastructures to support decision-making in food safety. Please see https://holifoodproject.eu/.

NutriAI: A mobile App built with Next.js, Directus, and React Native that uses AI to automatically track nutritional information from meals without manual entry. Users can take photos of their food, and the App recognizes the items and provides nutritional data. Please see https://www.nutriaiplatform.com/.

Nutrition-AI SDK by Passio AI: An AI software development kit that enables on-device food recognition for mobile Apps. It claims to recognize over 1.5 million foods and can be integrated into coaching, fitness, health, and lifestyle Apps. Please see https://www.passio.ai.

SnapCalorie: An AI-powered nutrition App that uses photo recognition to identify foods from meal pictures. It also includes an AI nutritionist chatbot that provides dietary guidance based on user's eating habits and goals. Please see https://www.snapcalorie.com/.

RxFood: A healthcare-focused nutrition App that integrates users' medical history with their dietary patterns to provide personalized nutrition recommendations powered by AI. Please see https://rxfood.co/.

LogMeal: An AI solution for automated food recognition, detailed nutrition analysis, and restaurant self-checkout using advanced food AI. Please see https://logmeal.com/.

Eaternity: Built towards optimal affordability and accuracy with goal of an accelerated transformation of the food industry. Product Environmental Footprint (Eaternity License) details scientific sustainability metric, based on food data, reducing CO₂ emissions, water footprint and whether you are certified for animal welfare, sustainable palm oil or soja. Please see https://eaternity.org/.

Naiya: A free AI nutrition tracker App for iOS that can recognize over 4,000 foods and estimate portion sizes from user-submitted photos. Please see https://naiya.en.softonic.com/iphone.

Bitewell: Bitewell is a company that has developed the FoodHealth Score, a personalized nutrition scoring system designed to simplify healthy eating. The FoodHealth Score evaluates foods on a scale of 0-100, accounting for nutrient density and ingredient quality. The system is tailored to chronic diet-related health conditions. Bitewell aims to help consumers make informed decisions about the foods they eat, potentially contributing to the prevention of diet-related diseases such as Type II Diabetes and heart disease. Importantly, Bitewell does not use any form of Artificial Intelligence in its processes and only aims to impact diet-related chronic conditions including high blood pressure, diabetes, and high cholesterol, not all chronic conditions and health goals. Please see https://www.bitewell.com/foodhealthscore.

The Drop: The Drop is an AI device that is worn as a necklace or shirt pin and is operated by Rex.Fit/BabylonAI Inc. It has a camera that uses AI to recognize food in front of the user and when it is eaten, automatically tracking foods in an accompanying app to provide the user with macros and micros for every meal eaten. Its goal is to make it easier and more streamlined for users to track what they eat so they can achieve nutrition goals easier. This is still in development stage and aims to launch early 2025. In short, this is another meal-tracking app that introduces AI image recognition to automatically track foods consumed by users. Please see https://getdrop.ai/.

Overall and despite numerous introductions and advancements in simplified nutritional tracking Apps, scoring and grading systems, several material challenges, risk gaps and real limitations remain. Nutritional tracking, grading or accounting systems often struggle with the dynamic nature and intersections of applied biosciences and purposeful food sciences, wherein new research can shift understanding of what constitutes a “healthy” relevant diet, even directionally, let alone personally. Additionally, the complexity of food matrices and the individual bioavailability metrics of discreet nutrients can make it difficult to accurately assess the prospective or intended health impacts of certain foods or other consumables. And moreover, cultural biases and regional dietary preferences can affect the applicability and acceptance of universal scoring systems. Critically and by design, the IRX FOODSCORE AI ecosystem overcomes each and all of these challenges for individuals or groups, target or afflicted populations, both domestically and globally by region or country.

In conclusion, efforts to develop universal relevant nutritional scoring or grading and holistic risk assessment systems have failed due to lack of purposeful nutrition and applied biosciences, which are crucial in helping consumers make informed choices, while also assisting manufacturers in creating healthier more purposeful products by intention and design. However, continuous refinement and adaptation of these systems are necessary to keep dynamic pace with scientific advancements and changes in so-called dietary guidelines. The integration of environmental and health assessments, as evidenced by our IRX FOODSCORE AI invention as a holistic purposeful food system approach, represents a material, actionable and scientific leap-forward through a dynamic AI-enabled, generative and ever-learning methodology that addresses the multifaceted impacts of food production and consumption for any global single consumer, or defined user or population, each and all user-aligned through relevant, analytical vantage points.

### Glossary of Terms

The following Glossary of Terms is meant to further define the referenced terms in the Field of the Invention section found on page 1 of this specification, and to describe, disclose and define the various technical terms as used throughout this specification more particularly.

(1) Proprietary System: Relevant Nutrition Scores (RNS) systematically integrates cutting-edge scientific methods and artificial intelligence (AI) to comprehensively assess and optimize the health impacts, purposeful opportunities and transparencies of risks associated with the intake of foods, consumables and ingredients, and their bio-functional impacts and efficacies. This advanced system leverages multiple AI technologies and data sources to provide nuanced, relevant nutritional and risk insights beyond traditional nutrition guidelines and scoring systems.

### Core Components:

A. Advanced Data Integration: The RNS AI system aggregates vast amounts of data from many diverse and global sources, including but not limited to:
   a) Scientific literature, clinical trial results and research studies (e.g., PubMed).
   b) Food composition databases, nutritional and ingredient content and disclosures.
   c) Preservative studies, disclosures and global health impacts databases.
   d) Crop pesticide and other food contaminant databases (raw and processed).
   e) Packaging composition, contaminant and health impact (e.g., leaching) databases.
   f) Global genomic and metabolomic data (studies of small molecules).
   g) Global, country-specific and culture-aligned consumer health and dietary data.

Machine Learning (ML) algorithms continuously process and analyze data to identify complex relationships by and between foods, medications, ingredients, sourcing, bioactive compounds, preservation practices (if any), packaging (types), preparation and user-aligned health outcomes.
B. AI-Powered Ingredient Analyses: Sophisticated RNS AI natural language processing and computer vision models are employed to:
   a) Extract detailed information on ingredients and processing methods from foods or product labels and specifications.
   b) Analyze molecular structure and properties: both natural and artificial ingredients.
   c) Predict interactions between ingredients and bioavailability, absorption, and efficacies.
C. Bioactive Compound Modeling: RNS AI system utilizes deep learning (DL) techniques to:
   a) Model the bio-functional effects of various compounds found in foods, beverages and any consumables.
   b) Predict synergistic or antagonistic interactions between bioactive components.
   c) Estimate the impact of food composition, processing, preservation, packaging and preparation methods on nutrient retention and bioactivity.
D. Personalized Nutritional Profiling: RNS AI advanced machine learning (ML) algorithms create personalized or user-specified, aligned and relevant nutritional profiles by considering:
   a) Individual/Target genetic variations affecting nutrient metabolism and efficacy.
   b) Gut microbiome composition and its influence on nutrient absorption.
   c) User-aligned and defined age, gender, health status, and lifestyle factors.
   d) Identified and purposeful dietary preferences and restrictions.
E. Dynamic Scoring Algorithms: The RNS AI system of sophisticated AI-enabled scoring algorithms to:
   a) Weigh multiple nutritional factors based upon the latest scientific evidence.
   b) Adapt to individual user profiles and stated health goals.
   c) Consider both short-term and long-term health impacts.
   d) Account for portion sizes, typical consumption and preparation patterns.

### Key Features:

A. Theoretical Food Formulation Optimization: The RNS AI system would enable diverse global users, consumers, food scientists and product developers to:
   a) Simulate the nutritional impact of different ingredient combinations.
   b) Optimize formulations for specific health benefits and target demographics.
   c) Predict potential sensory characteristics and consumer acceptance.
B. Real-time Adjustment Capabilities: Machine learning (ML) models continuously update based on new research findings or user feedback: Ensures RNS AI stays current and relevant for target products or populations, nutrition scores, risk profiling and claims compliance.
C. Explainable AI Integration: To enhance transparency and trust, the system incorporates explainable AI techniques to provide clear rationales for its scoring decisions and recommendations, all backed by scientific research and medical community evidence.
D. Regulatory Compliance Check: Integrated country-aligned regulatory databases (e.g., USA—FDA/USDA) and AI-powered compliance checkers ensure that all RNS AI scores and recommendations align with current food safety legislation and labeling regulations across any agencies, countries or jurisdictions. IRX AI Compliance and Risk Standards (CARS).
E. Applications: This IRX AI-enabled system revolutionizes various aspects of the food industry, and prevailing food practices, standards and legislation, including but not limited to:
   a) Product Development: Guiding the creation of healthier, more nutritionally optimized and purposeful food products, or improving existing products.
   b) Personalized/Target User Nutrition: Providing tailored dietary recommendations based upon user or individual health profiles and applied purposes.
   c) Public Health Initiatives: Informing policy decisions and nutritional guidelines, by systematically enabling new standards, policy implementation and industry-aligned nutritional exchanges, efficacy enablers and health directives.
   d) Consumer Education: Offering detailed, easy-to-understand nutritional information to consumers, realized through IRX FOODSCORE applications.

In summary, by leveraging the power of IRX AI and advanced scientific methodologies, this multi-dimensional ecosystem goes well beyond simple nutrient content analyses to provide holistic assessments of foods' health impacts and correlated medication efficacies, considering the many complex user-aligned interactions and individual variations. The resulting Relevant Nutrition Scores Artificial Intelligence Solutions (RNS AI) offer more comprehensive user and personalized approaches to unique nutritional evaluations, purposeful uses and health risks, transforming how users understand or optimize comprehensive health impacts of food choices.

(2) Advanced Scientific Methods: To fight diseases, optimize health & wellness, and to assess potential or realized nutritional impacts, RNS AI uniquely integrates advanced, disparate, ever-evolving AI-enhanced methods, deliverables and disciplines, leveraging diverse bodies of scientific knowledge and published medical research, dynamically considering user inputs, objectives, environments, cultures, lifestyles, life stages, genomic factors, and health influencers.
A. Schools of Sciences (including but not limited to):
   a) Behavioral Sciences: RNS AI incorporates behavioral science studies necessary to apply known or experiential impacts of human behavior on health. Understanding behaviors like diet, exercise, substance use, and lifestyle is critical for developing nutritional interventions to combat diseases or chronic conditions, while promoting healthy lifestyles, immunities and disease prevention.
   b) Biostatistics: RNS AI draws upon biostatistics to apply variable statistical methods to biological and health-related processes, opportunistically enhanced with intentional nutrition intake. It is essential for analyzing data & research to support evidence-based health care and dietary decisions.
   c) Crop Sciences: RNS AI leverages multi-disciplinary source inputs related to sustainable food production: plant biology, physiology, genetics, breeding, agronomy, environmental impacts, precision agriculture, climate-smart practices, post-harvest technologies, pesticides, and their respective impacts upon yields, quality, security, nutrition, resilience, and sustainability.
   d) Environmental Health: RNS AI integrates environmental impacts to health and how environmental factors affect human health, causal relationships and outcomes, not limited to effects of pollutants, climate change, and occupational hazards on health, and their influences over health situations.
   e) Epidemiology: RNS AI invokes epidemiology to integrate studies of how diseases affect health and illness of populations. It helps understand distribution and determinants of health-related states, events and outcomes, crucial for disease prevention and health promotion strategies.
   f) Exercise Sciences: RNS AI sources exercise science studies to assess impact of physical activities on health or disease, and the nutrition necessary to achieve objective health outcomes, and how exercise and nutrition combined can prevent chronic diseases, improve mental health, and enhance overall well-being while supporting individuals with physical ailments.
   g) Genomics and Personalized Medicines: RNS AI employs genomics necessary to apply learnings or studies of specific individual's genes and their interactive responsiveness to external or consumed influencers. Personalized nutrition may rely upon genomic clusters and related information to tailor purposeful diets and intentional nutrition to individual patients which RNS AI optimizes through multi-dimensional analyses and algorithms. This integrated nutrition approach helps in proactively fighting predicted disease risk and customizing treatments.
   h) Health Informatics: RNS AI exploits health informatics, involving information technology to collect, store, and analyze general, situational and specific health data. RNS integrated health informatics support evidence-based practices, health promotion, disease prevention and large dataset insights.
   i) Interdisciplinary Health Care: RNS AI assessments of interdisciplinary healthcare data involve collaboration among various health professionals (general and specific) to provide comprehensive care, inclusive of nutrition. This approach improves patient outcomes by addressing integrated multifaceted natures of health and disease.
   j) Nutritional Sciences: RNS AI leverages nutritional science research, proactively applying impacts of foods and nutrients upon health and disease. RNS considers various aspects of metabolism, dietary patterns, and the role of all nutrients in disease prevention and health promotion. Understanding the nutritional needs of different lifestyles (opt-in or imposed), varying life stages and in different cultural contexts is essential for optimizing health.
   k) Pharmacogenomics: RNS AI screens pharmacogenomics studies as to how genes affect a person's response to drugs naturally, combining dietary and nutritional interactions and bio-availabilities. RNS AI helps to assign personalized nutrition plans in support of medication plans, effectiveness and fewer side effects.
   l) Physiology: RNS AI embeds physiological sciences and disciplines useful for fighting diseases, maintaining health, supporting wellness, assessing nutritional impacts, and optimizing objectively healthy lives. RNS AI focuses on applied functions and mechanisms of living organisms, fundamental to comprehending how bodies maintain health and respond to various stressors and diseases.
   m) Psychoneuroimmunology: RNS AI incorporates psychoneuroimmunology to explore and apply interactions between nervous systems, immune systems, and psychological processes. Understanding interactions can help in developing strategies to manage stress and improve mental health, supporting overall wellness.
   n) Public Health: RNS AI pulls global public health sources to focus on protecting and improving health of select cultures, countries, or communities through integration of public policies, related to local or regionalized nutrition and available foods, preparation, ingredients, storage, claims, distribution, sales and consumption, as well as related research for disease or injury prevention. This provides for a proactive multidisciplinary approach, including biostatistics, historical health sciences and observations, incorporating both short- and long-term environmental impacts to health outcomes.
   o) Social and Cultural Health Sciences: RNS AI uses social disciplines and cultural health sciences to evaluate impacts of social influencers and cultural factors on overall health and nutrition, This includes, but is not limited to, data drivers and related research on social determinants of health such as income, education, social support, and access to culturally aligned, high quality foods. This approach is necessary for addressing disease, health and wellness determinants, and for promoting reactive or proactive nutrition in support of social and culturally aligned health outcomes.
   p) Systems Biology: RNS AI subscribes to systems' biology by opportunistically integrating biological data for the purpose of harnessing complex nutritional interactions within diverse human biological systems. This intentional inclusion is essential for promoting purposeful nutrition and scientific wellness on behalf of select users, unique individuals, and target populations and communities. This allows RNS AI to actively apply “big data” to generate actionable nutrition-inspired insights and applications to promote wellness objectives, disease prevention, intended immunities, and health outcomes, each and all as applied from published public or private studies of bodies, brains and gut microbiomes.

In summary, advanced sciences serve as a cornerstone to IRX FOODSCORE and resulting RNS AI-related health and relevant nutrition optimization scores and actionable deliverables, defining opportunity, function, purpose and risk.
B. Applied and Functional Sciences (including, but not limited to):
   a) Genomics and Personalized Medicine: RNS AI adapts to physiological responses at genetic levels, helping tailor purposeful nutrition and complementary dietary plans as integrated components to comprehensive medical care for individuals/target populations.
   b) Health and Disease Mechanisms: RNS AI uses this field to help differentiate between mechanisms of health and disease, facilitating the development of nutrition strategies to promote health and to complement disease treatments effectively, mitigating side-effects.
   c) Homeostasis and Resilience: RNS AI assesses how a body maintains homeostasis (bodily systems balance and function) and adapts to stressors leveraging nutrition reactively or proactively, which is crucial for maintaining health and preventing diseases.
   d) Interplay with Nutrition and Environment: RNS AI examines the interactions and responsiveness between environmental factors, nutrition, exercise, and disease, providing insights into how these distinct elements influence human function and health.
   e) Mechanistic Understanding: RNS AI uses physiological sciences to enable mechanistic understandings of how various systems in the body work, from cellular to whole-system levels, which is essential for developing targeted nutrition interventions and treatments.
   f) Nutritional Science: RNS AI incorporates diverse physiological insights; crucial components for understanding how nutrients affect bodily functions and overall health.
   g) Public Health and Epidemiology: RNS AI weights impacts of epidemiological studies, which provide a deeper understanding of disease mechanisms and health maintenance, all combined and interacting with bodily functions driven by required nutrition consumption.
   h) Systematic Biology: RNS AI relies upon physiological sciences and systems biology as we assess complex interactions activated by nutrition within biological systems to drive health and purposeful outcomes.

RNS AI integrates advanced scientific methods and disciplines into a multidisciplinary approach necessary to optimize health, prevention and natural immunities through purposeful, functional nutrition, or alternatively to support medication efficacies, treatments and remissions. Processing disparate data related to how products and bodies function under various conditions is critical for developing effective proactive or responsive nutritional strategies. This approach is essential for determining weighted nutrient relevance, impacts and tailored diets for disparate users, cultures, environments, lifestyles, life stages, and genomic factors, each and all necessary to fight diseases and chronic issues, or to optimize health. By integrating knowledge from advanced scientific disciplines, RNS AI develops and applies dynamic, holistic nutritional strategies (proactively and reactively) as required to fight diseases, support health & wellness, and to assess purposeful nutrition impacts; optimizing health for diverse global user populations.

In conclusion, our methods enable IRX and RNS AI-enhanced deliverables, providing the foundations of a proprietary IRX Infrastructure as a Service (IaaS), while delivering key RNS AI components:
a) IRX AI cloud-based computing model. (Clouds: Private, Public, Hybrid and Multi).
b) Provides virtualized computing resources over internet on a Pay-as-you-go basis.
c) Provides functional foundation to leverage IRX FOODSCORE and RNS AI to assess current consumables, foods, pharmaceuticals and future products based upon common exchange of understanding of transparent opportunities and risks, short and long-term.
d) Allows organizations to rent essential IRX IT infrastructure with SaaS or other services.
e) Components. e.g., servers, storage, data sourcing, networking, software applications, etc.
f) IRX FOODSCORE services and Relevant Nutrition Scores (RNS) are leveraged across diverse user communities to provide relevant health impacts, assessments and insights.

(3) AI-enabled Software and Applications (Tools): To fight diseases, optimize health and wellness, and to assess potential for purposeful nutrition(al) impacts, RNS AI integrates and leverages general and generative AI-enabled diverse, disparate and ever-evolving systems, software, tools and technologies. RNS AI creates sophisticated nutritional analyses, assessment and scoring systems to optimize user-aligned, productized and commercialized nutrition impacts, weighted influencers, health opportunity and risk-scored content in support of stated health objectives, imposed obstacles and desired outcomes.
A. Nutritional Analysis Software (Sample RNS AI systematic software and application types being evaluated; May license, acquire, develop and deploy any or none of the following):
   a) Cronometer: RNS AI may leverage comprehensive food tracking tools, monitoring macros, micros & other health metrics, capitalizing on extensive UPC/product databases, target goal setting, integrated food scoring, nutrition tracking, nutritional intake planning.
   b) ESHA Food Processor: RNS AI may use this or other nutrition analysis software combining existing and extensive food or ingredient databases with user-friendly interfaces to analyze 172+ select nutritional components, constructive dietary impacts, prospective meal nutrition, including commercial reporting features.
   c) myfood24: RNS AI may utilize this robust nutrition platform enabling accurate nutrient analysis of 220+ nutrients; supporting dietary insights, applied research.
   d) Nectar for Dietitians: RNS AI may employ this nutrition software to assess nutrition deficiencies and analyze purposeful nutrition opportunity gaps with potential to improve individual dietary habits and nutrition goals.
B. Data Management and Analysis Tools: Data Warehousing and Storage (Examples)
   a) Amazon Redshift: A fully managed data warehouse service in the cloud, optimized for large-scale RNS AI data storage and analysis.
   b) Google BigQuery: A serverless, highly scalable, and cost-effective multi-cloud data warehouse designed for RNS AI business agility.
   c) ALTAIR (AI, Data Analytics and Technologies): RNS AI Data preparation and sourcing solutions enable extraction, cleansing and transformation of RNS AI data in different forms and multiple sources. (ALTAIR has knowledge, tools and ability to deliver many of the RNS AI capabilities required).
C. Data Cleansing and Aggregation (Examples, including but not limited to):
   a) Talend: An open-source data integration platform that provides RNS AI-defined tools for data cleansing, transformation, and aggregation.
   b) Informatica: A data integration tool that offers RNS AI comprehensive data management solutions, including data cleansing and aggregation.
D. Data Extraction (Examples, including but not limited to):
   a) Apache NiFi: A data integration tool that supports powerful and scalable directed graphs of RNS AI data routing, transformation, and system mediation logic.
   b) Alteryx: A data analytics platform deployed for RNS AI that provides data preparation, blending, and advanced data analytics capabilities.
E. Advanced AI, Computing, Algorithm Tools (Examples, including but not limited to):
   a) xAI Colossus, other RNS AI training clusters; systems such as Stargate or others: Microsoft—OpenAI, Meta, Google, Other AI training infrastructures.
   b) IBM Qiskit Quantum Computing: Open-source quantum computing and RNS AI software development framework(s).

Features: Building, optimizing, visualizing quantum circuits, AI-powered optimization of RNS, simplified execution modes; serverless tools for quantum-centric supercomputing. Applications: To be used for developing RNS AI next-generation quantum algorithms and integrating classical and quantum computing resources.
F. Machine Learning & AI Tools (Examples, including Google Cloud Platform below):
   a) TensorFlow: An open-source platform for RNS AI machine learning (ML), used for building and deploying machine learning models.
   b) PyTorch: An open-source machine learning library (Torch library), used for applications: computer vision and natural language processing support of RNS AI.
G. Algorithm Development (Examples, including but not limited to):
   a) MATLAB: A high-level language and interactive environment, potentially used to support RNS AI numerical computation, visualization, and programming.
   b) R: Potentially utilized RNS AI program language, software environment, statistical computing, graphics.
   c) PYTHON: A general-purpose programming language that may be utilized to support RNS AI target tasks, including but not limited to automation, data analyses, data science, software or web development and scientific computing.
H. Cloud Computing Platforms (Examples, including but not limited to):
   a) Amazon Web Services (AWS): Full cloud platform; 200+ fully-featured services. Applications: RNS AI may use AWS for computing power, storage, and content delivery.
   b) Google Cloud Platform (GCP): GCP is a suite of cloud computing services, running on same infrastructure that Google uses internally for end-user products. Applications: RNS AI may use GCP computing, data storage, analytics and machine learning (ML).
   c) Microsoft Azure: A cloud computing service created by Microsoft for building, testing, deploying, and managing applications and services. Applications: RNS AI may use Azure services; computing, analytics, storage & networking.
I. AI Data Visualization and Reporting Tools (Examples, including but not limited to):
   a) Tableau: Data visualization tool; converting raw data into understandable format. Features: Supports RNS AI Interactive and shareable dashboards, real-time data analysis, and integration with various RNS AI data sources.
   b) Microsoft Power BI: Business analytics service by Microsoft that provides interactive visualizations and business intelligence capabilities. Features: RNS AI may use for data preparation, data discovery, and interactive dashboards.
   c) Others Considered: Based upon function, source and dashboards, RNS AI considering: Akiko, Qlik, C3.js, Looker, Sisense, Polymer, Akiko, Zoho, ChartBlocks, Plotly, Geckoboard.

(4) Advanced Algorithms: RNS AI advanced algorithms optimize purposeful nutrition proactively (food planning purposes) or reactively (score foods and grade existing diets) for various specific or compounded health conditions, considering key individual or group objective factors (general disease or opt-in medical records disclosures); following general (evolutionary) stages & steps:
A. RNS AI Core System Components:
   a) RNS AI Data Collection: Gather relevant information; target users, individuals and groups, including (but not limited to):
      i. Age, gender, height, weight, activity level
      ii. Health status, medical conditions, pre-existing allergen, preventative, other?
      iii. Familial and genetic predispositions and preventative concerns
      iv. Cultural backgrounds and related dietary preferences
      v. Societal lifestyles, religions, or other influential factors
   b) RNS AI Nutritional Needs Assessment: Calculate baseline nutritional drivers from:
      i. Basal metabolic rate (BMR); estimated or actual
      ii. Macro and micro-nutrient needs or objectives (proactively or reactively)
      iii. Specific health conditions, concerns, objectives, goals (short and long-term)
   c) RNS AI Food Database Integration: Utilize comprehensive food databases with:
      i. Macro, Micro and Phyto/Nano nutritional composition of target foods.
      ii. Cultural and regional food options and guidelines; personalized restrictions.
B. RNS AI General Optimization Process:
   a) RNS AI General Constraint(s) Definition: RNS AI set parameters based upon:
      i. Dietary restrictions (e.g., vegetarian, kosher, halal, etc.)
      ii. Allergies, intolerances or preventative objectives
      iii. Cultural or Religious preferences; personal limitations
      iv. Health condition-specific objectives or requirements
   b) RNS AI Targeted Nutrient Balancing: Optimize nutrient intake by:
      i. Setting macronutrient targets by distributed sources
      ii. Optimizing micronutrient and Phytonutrient target(s) by source(s)
      iii. Dynamically evaluating; considering nutrient interactions and bioavailability
   c) RNS AI Product or Nutrient Planning: Proactively or Reactively based parameters
      i. Meet target nutritional requirements (AI-enabled min./max.) analyses
      ii. Alignment with individual preferences, limitations or exclusions set
      iii. Provide variety and balance; considering likes or dislikes, filters, or priorities
   d) RNS AI Scoring and Grading: Evaluate existing or proposed product(s) based on:
      i. Purposeful nutritional alignment, PubMed researched impacts or responses.
      ii. Adherence to specific health objectives or disease-aligned dietary guidelines.
      iii. User-aligned predispositions, preferences, restrictions, objectives.

### C. RNS AI Adaptive Elements: (Individuals or Groups)

a) Input and Feedback Integration: RNS AI incorporates general and/or user feedback to:
   i. Adjust recommendations based on general guidelines or personal preferences.
   ii. Confirm and address any issues or concerns; predisposition or preventative issues.
b) Continuous Learning: RNS AI dynamically updates recommendations based upon:
   i. AI-enabled or other technology inspired functions or findings.
   ii. New scientific research, Eastern/Western medical, nutrition, or interaction studies.
   iii. Changes in individual health status or product goals (preventative or reactive).
c) Personalization: RNS AI refines recommendations over time; target user, individual, group or product:
   i. Analyzing patterns or responsiveness in user or product behavior and preferences.
   ii. Incorporating data directly input and/or from wearable devices or health trackers or other assessment technologies.
   iii. Incorporating opt-in data and disclosures (product or person related).

RNS AI algorithms employ machine learning (ML) techniques for predictive modeling inclusive of but not limited to decision trees, neural networks, or genetic algorithms, necessary to optimize recommendations based upon dynamic or defined complex sets of variables and constraints. Quantum computing (QC) will also be employed as practical and available.

RNS AI systems dynamically employ ML models (2 types)—Classification and Regression:
a) RNS AI Classification modeling:
   i. Narve Bayes: classifies new data based upon highest probabilities of each feature. RNS AI applies this to correlation between foods, medicines and health.
   ii. k-Nearest Neighbor (KNN): classifies objects and data based upon proximity and similarities, leveraging distance metrics. RNS AI applies KNN to assess similar or dissimilar products, health issues, nutrients, bio-responses, etc.
   iii. Discriminant Analysis: classifies data and classes based upon linear combinations Gaussian Distributions. RNS AI utilizes this approach to train ML models to find linear or quadratic parameters and boundaries of new data.
b) RNS AI Regression modeling:
   i. Linear or non-Linear Regression: statistical modeling and training techniques utilized to assess data, predictors, variability, continuous responses and relationships between similar and disparate data (linear and non-liner). RNS AI leverages these approaches when first assessing new datasets and sources. RNS AI also deploys a Generalized Linear Regression modeling approach, which differs from the two above, in order to determine a best fit function.
   ii. Gaussian Process Regression (GPR): A probabilistic, non-parametric modeling technique used for regression tasks. GPR is a powerful and flexible approach that RNS employs to analyze complex datasets and make predictions with quantified uncertainty. Key aspects of GPR in RNS AI include:
      a. Probabilistic framework: GPR provides a distribution over possible functions that fit the data, allowing RNS AI to capture uncertainty in its predictions.
      b. Kernel-based approach: RNS AI uses kernel functions to define covariance between data points, enabling it to model various types of relationships or patterns in data.
      c. Non-parametric nature: Unlike linear regression, GPR doesn't assume a fixed functional form, allowing RNS AI to model complex, non-linear relationships without specifying them explicitly.
      d. Uncertainty quantification: GPR provides both a mean prediction and a measure of uncertainty, which RNS AI uses to assess the reliability of its predictions and guide decision-making.
      e. Prior knowledge incorporation: RNS AI incorporates domain knowledge into the GPR model through the selection & customization of kernel functions.
      f. Adaptive modeling: As new data becomes available, RNS AI updates its GPR model, refining predictions and uncertainty estimates.
      g. Hyperparameter optimization: RNS AI optimizes GPR model hyperparameters to best fit the data, improving prediction accuracy.

RNS AI also utilizes techniques like constrained optimization to balance multiple competing objectives, such as meeting nutritional needs while adhering to dietary preferences and restrictions. The main RNS AI goal is to provide personalized or product-aligned, evidence-based nutritional guidance or feedback (relevant scores and grades of purposeful nutrition) tailored to a target product's, individual's or group's unique use circumstances or objectives, promoting optimal relevant health outcomes, all while respecting cultural, religious, personal preferences, predispositions, or situational circumstances. RNS AI advanced algorithms leverage disparate data transformation frameworks to process lifestyle, life stage, disease and health-relevant nutrition in support of specific objectives or challenges.

Data transformation frameworks provide a foundation for processing and analyzing health and nutrition data. RNS AI key frameworks include:
A. RNS ETLIELT Tools: RNS AI may deploy and use Informatica, Alteryx, ALTAIR, Fivetran; cloud-based solutions.
Extract, Transform, Load (ETL) and Extract, Load, Transform (ELT) tools are essential for preparing health and nutrition data for analysis. These frameworks allow for:
   a) Data extraction, various sources (e.g., medical records, nutrition databases, etc.).
   b) Cleaning and standardizing RNS data.
   c) Loading data into RNS AI analytical systems.
B. RNS AI Machine Learning (ML) Frameworks: Crucial for developing RNS AI predictive models and analyzing diverse, complex, user-aligned and individualized health data and nutrition patterns.
   a) TensorFlow: Google-developed; RNS AI can leverage for deep learning; neural networks.
   b) PyTorch: RNS AI may use for research; developing custom neural network architectures.
   c) Scikit-learn: Python library; may be used to develop classical RNS AI machine learning algorithms.

RNS AI frameworks enable the creation of sophisticated RNS nutrition optimization models and health outcome predictions. RNS AI employs many types of algorithms which enable relevant health, purposeful nutrition, medical research and other disparate types of data transformation. Many types of algorithms are used to transform health and nutrition data into actionable insights:
C. RNS AI Types of Algorithms Employed: (inclusive of, but not limited to)
   a) RNS AI Clustering Algorithms: RNS groups similar data points together, which are useful for:
      i. Identifying RNS AI projected nutrition patterns associated with specific health outcomes.
      ii. Grouping afflicted people or treated patients with similar health profiles (macro). RNS samples used: K-means clustering and Hierarchical clustering.
   b) RNS AI Classification Algorithms: RNS AI predicts categories or labels applied to:
      i. RNS AI predicting disease-risk; based upon dietary and nutrition habits (predictive).
      ii. RNS AI categorizing actual or theoretical foods; target nutritional content; macro/micro/Phyto.
RNS samples used: Random Forests, Support Vector Machines and Neural Networks.
   c) RNS Regression Algorithms: RNS AI predicts continuous values, useful for estimating impacts of specific nutrients on health markers, prevention, responsiveness & outcomes:
      i. RNS AI predicting calorie density and nutritional intensity needs based upon unique individual or group characteristics, and the corresponding RNS predictive impacts and scores.

RNS AI Algorithm Examples: Linear regression, polynomial regression, gradient boosting.
d) RNS AI Natural Language Processing Algorithms (NLP): NLP algorithms used to:
   i. Extract RNS AI relevant health or nutritional information from unstructured medical texts (captures and transcribes raw data).
   ii. RNS AI enables ability to analyze food diaries, nutritional descriptions, other files.

RNS AI techniques: Named entity recognition; Sentiment analysis used in this context. Overall, RNS AI deploys progressive AI-enabled algorithms, nutrition(al) processing and health optimization and scoring engines, optimizing purposeful foods and diets for specific user-aligned health objectives, key issues or concerns, proactively or reactively, achieved (but not limited) by:
A. RNS AI Relevant Data Collection and Integration: RNS AI gathers relevant data from various sources, including but not limited to medical records, nutritional databases, and wearable devices.
B. RNS AI Data Cleansing & Normalization: RNS AI standardizes units, fill missing values, remove outliers.
C. RNS AI Feature Engineering: RNS AI crafts relevant raw data features; nutrient ratios, frequency metrics.
D. RNS AI Personalization: RNS AI applies machine learning (ML) algorithms; individual patterns and preferences.
E. RNS AI Health Goal Mapping: RNS AI defines clear, measurable health objectives based upon specific diseases, health issues, or preventative goals or proactive health objectives.
F. RNS AI Nutrition Optimization: RNS AI deploys optimization algorithms (e.g., genetic algorithms, linear programming) to score or create foods that meet nutritional requirements and health goals.
G. RNS AI Outcome Prediction: RNS AI utilizes predictive models to estimate the actual or potential impacts of nutritional changes on proactive or reactive health outcomes for specific health factors.

Critically, RNS AI algorithms source many global research systems (GRS) by leveraging the diverse technologies defined herein to access and extract many major and disparate data and research sources by types of information, either directly or indirectly, and related to or influences upon nutrition, health, and disease, which are summarized and categorized by RNS AI as follows:
A. RNS AI Academic and Scientific Databases:
   a) PubMed/MEDLINE: RNS AI utilizes holistic biomedical literature databases to access 30 million+ citations/abstracts (and growing), covering, but not limited to:
      i. Biochemistry/metabolism: metabolic diseases given age, life stage, culture, +
      ii. Clinical trials: Disease, disorders, allergens, physical/mental impairments, +
      iii. Epidemiology: population distributions, patterns, health/disease determinants
      iv. Nutritional sciences: biochemical, physiological processes, and food impacts on life (user-aligned or defined)
      v. Public health interventions: evidence-based methods for specific public health goals, preventions and responses to foods, diseases, environment, other, etc.
      vi. Systematic reviews and meta-analyses: process evidence related to specific health situations or conditions (actual or hypothetical).
   b) Cochrane Library: RNS AI leverages this database which focuses on systematic reviews and meta-analyses of healthcare interventions, including nutritional studies and impacts.
   c) Web of Science: RNS AI leverages this multi-disciplinary database to index high-impact journals across scientific fields, including nutrition and health sciences, with a diversity of applications.
B. RNS AI Government and International Organization Resources:
   a) World Health Organization (WHO) Global Health Observatory: Provides RNS AI with data on global health indicators, including nutrition-related statistics.
   b) National Institutes of Health (NIH) Databases: RNS AI sources NIH specialized databases, trials, publications and journals:
      i. ClinicalTrials.gov: ongoing clinical trials
      ii. Dietary Supplement Label Database
      iii. DailyMed: drug labeling information
   c) WHO Global Health Observatory: Provides RNS AI with data on global health indicators, including food, crop and nutrition-related statistics.
   d) FAO/WHO GIFT (Global Individual Food Consumption Data Tool): RNS AI uses to harmonize data: individual food consumption, food security, evidence-based policies of The Food and Agricultural Organization of the United Nations (FAO).
C. RNS AI Professional Organizations and Societies:
   a) American Society for Nutrition (ASN): Publishes multiple nutrition journals; hosts annual conference showcasing cutting-edge nutrition research used by RNS AI.
   b) Academy of Nutrition and Dietetics: Evidence-based nutrition guidelines & papers leveraged as applicable by RNS AI.
D. RNS AI Research Types and Study Designs: RNS AI leverages any or many of the following:
   a) Any published studies related to randomized controlled trials (RCT's), random experiments, treatments and control groups.
   b) Cohort studies: studies groups over time, assess incidence & disease cause.
   c) Case-control studies: retrospective observational conditions vs. no controls
   d) Cross-sectional surveys: population data; point in time vs outcome prevalence
   e) Metabolic studies: controlled feeding trials; measurements of metabolic responses
   f) Nutrigenomics research: genetic variations/responses, nutrients, dietary patterns
   g) Microbiome studies: how microbials affect health, disease, and responses to diet.
E. RNS AI Select Research Areas (Including but not limited to studies and sources):
   a) Disease Prevention and Treatment—Studies examining role of nutrition impacts Examples: Cardiovascular, Type 2 diabetes, Cancers, Obesity, Neurodegenerative and any other disorders or disease states, multiple diseases, and any chronic health impairments.
   b) Nutrient Bioavailability & Interactions—Country, culture-aligned research Examples Only: Nutrient absorption mechanisms, Food matrix effects, Nutrient-drug interactions, Nutrigenetics and personalized nutrition, genomic clusters, etc.
   c) Public Health Nutrition—Studies regarding:
      i. Food fortification programs, School nutrition interventions, Community-based nutrition education, Culturally aligned and ethnic foods, etc.
   d) Integrative Approaches—Country, culture-aligned research combining nutrition with other impacting lifestyle influencers:
      i. Physical activity intervention; Stress reduction techniques; Improved sleep, +
   e) Specialized Nutrition—Country, culture-aligned study focused applications of:
      i. Sports, Geriatric and Pediatric nutrition, and nutrition for critical illnesses, +
   f) Food Systems and Sustainability—research studies, trials and data governing:
      i. Sustainable diets (aligned by countries and cultures), Food and crops' security by country and cultures; country-aligned climate change impacts on nutrition.
   g) Emerging Research Tools and Methodologies (individual upload, opt-in data):
      i. Metabolomics and proteomics, AI in nutrition research, Big data analytics in nutritional epidemiology, and Wearable devices for dietary assessment.

By leveraging diverse data research sources and methodologies, RNS AI gains a persistent, dynamic, and comprehensive understanding of complex relationships between nutrition, health, and disease prevention and treatments. Ever-evolving research is a cornerstone of user-select food scores, purposeful nutrition and personalized recommendations for target health conditions or proactive objectives, cultural priorities, and life stages and lifestyles (opt-in or imposed).

In summary, ever-evolving RNS AI-enabled algorithms fuel nutritional data refineries, scientific methods, user-adaptive software and dynamic application environments, which provide for a proprietary IRX Platform as a Service (PaaS) and other services (e.g., Software as a Service or SaaS), enabling many RNS AI deliverables, including but not limited to the following:
a) IRX cloud-based computing models. (Clouds: Private, Public, Hybrid and Multi)
b) Providing subscriber developers with a complete and ever-evolving nutrition and actionable impacts and insights environment to build, deploy, and manage IRX AI applications without complexities of managing underlying infrastructure.
c) FOODSCORE Nutritional Data Refinery/ETL Tools. (System, methods, software)

(5) Health Impacts (Types & descriptions below): RNS AI integration of artificial intelligence (AI) into user evaluations and scientific-based health impacts of foods and nutrition (transformative approach) purposefully leverages advanced scientific disciplines, technologies, and global research. The RNS AI-enabled evaluation system is designed to assess and optimize the many specific observed or likely realized health impacts of foods and nutrition across diverse users, populations, considering variables such as culture, climate, environment, age & life stage.

RNS AI relies upon diverse AI technologies (e.g., generative), including but not limited to machine learning (ML) and deep learning (DL), which increasingly analyze diverse datasets from food composition databases, nutritional studies, and health records. RNS AI leverages these ever-evolving technologies to help us understand complex diet-disease relationships and responsiveness, and for personalizing nutritional advice or RNS AI-proprietary relevancy scoring of foods based on an individual's objectives or target health profiles, genetic information, dietary drivers or socio-cultural preferences.

RNS AI sources and processes disparate global user, food, medical, scientific and other data in order to identify food impacts and medication interactions, efficacies and applied effects of named nutrients within products or upon user-relevant health. Specifically, RNS assesses the direct and derived impacts of pesticides, preservatives, processing, production and packaging upon global crops, ingredients, foods, supplies, consumption and health. Importantly, RNS AI systems facilitate an evolutionary and revolutionary monitoring, learning and evaluation of distinct dietary impacts by integrating all available disparate data from diverse data sources related to foods, nutrients, environments, crops, conditions, cultures and dietary practices. RNS AI allows for a holistic, nuanced understanding of how different foods and distinct nutrients (natural/artificial) impact, affect and optimize specific health outcomes for any health context, incidental or intentional.

The use of RNS AI in this domain supports product relevancy scoring (nutrition-driven, health objective or impairment-responsive) and user-aligned, individualized or target group development of product, personalized or group-purposeful nutrition impacts or plans, adaptive to dynamic health drivers or influencers, and in consideration environmental factors and other evidence-based impacts. The RNS AI applications of AI in nutrition extends to predictive modeling, wherein AI algorithms predict potential health influencers and outcomes based upon prospective, actual, observed or theoretical product(s) consumption or dietary patterns, and identifies potential preventive or responsive measures to mitigate health risks (risk profiling), support health objectives and claims compliance, or to optimize efficacy of foods and medications. This capability is crucial for addressing product, individual and public health challenges, allowing for purposeful nutrition, product formulations, tailored interventions, unique dietary needs, and health risk mitigation of target populations.

In summary, RNS AI attributes complex scores and grades to actual or prospective health impacts and relevancy. This enables users, people, products or brands (existing/theoretical), to dynamically evaluate potential impacts and outcomes (good and bad), with purposeful or circumstantial consumption of foods or nutrients in any form(s), and in consideration of all available research, data and influencers upon likely biological responses or outcomes, necessary for purposeful nutritioning. A persistent and ongoing RNS AI-enabled evaluation (score or grade) of foods, nutrients and products (actual, artificial or theoretical) assesses relevant and contextual influences upon objective health impacts, which signifies a momentous advancement and re-definition of the field of applied nutritional sciences. RNS AI combines cutting-edge nutritional technologies with comprehensive, up-to-date data analyses to optimize health by addressing the intricate interplay and impacts of consumables, diets and medications upon existing or future populations, at any place or time.

(6) Existing Consumables and Theoretical Consumable Formulations (Types & descriptions below):
A. Food, Nutrition, and Ingredients: Summary Overview:
   a) RNS AI assesses consumables, including but not limited to foods, wherein foods are a fundamental necessity for living organisms, providing essential nutrients required for energy, growth, and health. Foods encompass many substances consumed in different forms: raw, processed, or cooked; across all cultures and regions.
   b) RNS AI defines food (existing or theoretical) as any substance consumed proactively or reactively to provide accidental, incidental or intentional nutrition or energy to any person. Foods are typically derived from plants, animals, or fungal sources and contain essential nutrients: fats, carbohydrates, proteins, vitamins, and minerals, which are ingested and assimilated by cells of any person or organism to maintain life, stimulate growth, and for energy. However, RNS AI postulates that foods can be ever-more purposeful beyond their traditional basic needs and uses, casual or intentional.
   c) RNS AI recognizes that nutrition in its simplest form refers to the process by which organisms take in and utilize food substances. The many nutrients found in food are crucial for various bodily functions, including the growth, repair, and maintenance of bodily tissues and organs, as well as the regulation of vital functions and processes.
   d) RNS AI recognizes and leverages core nutrients (macro, micro, Phyto and nano nutrients):
      i. Carbohydrates: Necessary to provide energy; RNS AI optimizes purposeful blends.
      ii. Proteins: Essential for growth & repair of tissue; RNS AI reaffirms sources & quality.
      iii. Fats: Concentrated energy source necessary for absorption of certain vitamins.
      iv. Vitamins and Minerals: Support biochemical processes; contribute to objective health.
   e) RNS AI assesses all ingredients within foods, as well as their sourcing and processing:
      i. Ingredients are individual components used in preparation of a food (one or many).
      ii. Ingredients can be raw or processed, typically undergoing changes from natural states through varied methods: washing, cooking, freezing, adding preservatives. Processing varies, yet often affects the nutritional content of food, as some nutrients are lost during processing, while others may be added to enhance specific nutritional values.
   f) RNS AI considers and assesses all types of distinct food preparation and processing:
      i. Unprocessed Ingredients or Minimally Processed Foods: Include fresh vegetables fruits, grains and meats that have not been significantly altered from an original state.
      ii. Processed Culinary Ingredients: Includes oils (See Section 18 for specific oil uses, benefits and risks), sugars, salts that are used in cooking.
      iii. Processed Foods: Foods that have been altered for convenience or preservation, such as canned vegetables or cured meats. (often use salts).
      iv. Ultra-Processed Foods: Highly processed foods with added sugars, fats (including but not limited to oils; See section 18: oil uses, benefits and risks) and artificial ingredients (colors, flavors, preservatives); often linked to negative health outcomes.
   g) RNS AI considers all cultural, regional and religious variations, as well as global influences:
      i. Food consumption patterns vary widely across different cultures, regions, and religions influenced by geography, climate, economics, convenience trends, used natural and artificial ingredients, and cultural traditions. Globalization, preservation, exportation and importation, fast food trends, expanded food processing, and worldwide accessibility and convenience measures have had material impacts.
      ii. Food is essential for life and for providing nutrients which directly impact health & well-being (physical, mental, spiritual, etc.).

(7) Artificial and Natural Ingredients (Types & descriptions below): RNS AI recognizes that food ingredients are broadly categorized into both artificial and natural types with unique sources, processing and applications within a food industry. Understanding distinctions is a cornerstone for RNS AI to score and grade health-influenced impacts and uses.
A. Artificial Ingredients: Synthetic food colors and additives are used to enhance appearance, taste, and shelf life of food products. However, they can have adverse health effects as listed below and including but not limited to:
   a) Artificial Ingredients: Synthesized in laboratories and are designed to mimic or enhance natural ingredient properties; Often used for cost-effectiveness, stability, and consistency.
   b) Artificial Flavors: Chemically synthesized to replicate natural flavors are used because they are cheaper and more stable than their natural counterparts. Crafted by analyzing molecular structures of natural flavors, recreating them with synthetic chemicals.
   c) Artificial Sweeteners: Sweet substances like aspartame, sucralose and saccharin provide sweetness without calories that natural sugars have; used in diet or low-calorie products.
   d) Color Additives: Synthetic colors, like FD&C Red No. 40, Yellow No. 5 and Blue No. 1, are used to enhance or restore color in foods. They are often more vibrant and stable than natural colorants but are also often associated with cancer and other health issues.
B. Natural Ingredients: Natural is often misleading; no strict definitions or enforcement as listed below and including but not limited to:
   a) Natural Ingredients: Extracted or derived from plant, animal, or mineral sources and are minimally processed. They are often perceived as healthier or more authentic, yet this is not always scientifically substantiated. RNS AI specifically assesses this use, validity of perceived claim and potential impacts, good or bad, and relevant for inquirer.
   b) Natural Flavors: Extracted from natural sources such as fruits, vegetables, herbs, spices, and animal products. Common natural flavors include amyl acetate (from bananas), citral (from citrus fruits), and benzaldehyde (from almonds). Despite being labeled as “natural”, these flavors can be highly processed and contain numerous chemical additives.
   c) Natural Sweeteners: Include sucrose (table sugar), fructose (fruit sugar), and honey, each used to add sweetness to foods & beverages without the use of synthetic chemicals.
   d) Color Additives: Natural colorants derived from sources like fruits, vegetables, and spices. Examples: beta-carotene (carrots) and annatto (from seeds of the achiote tree).

C. Common Uses and Safety: RNS AI realizes that both artificial and natural ingredients are used extensively across various food products and types, from beverages and snacks to baked goods and processed foods. The safety of these ingredients is regulated by country-aligned agencies like the U.S. FDA, which requires that all food additives, whether artificial or natural meet specific safety standards. Overall, artificial and natural ingredients and flavors may be chemically similar, but labeling often influences consumer perceptions and decisions. Artificial ingredients are typically favored for cost-effectiveness & consistency, especially in large-scale food production, whereas so-called natural ingredients or flavors are typically preferred as a claimed less-processed food option.

(8) Associated Bio-functional Effects (Types & descriptions below): The RNS AI assessment of the nutritional weighting of artificial and natural ingredients in consumed foods involves a complex process that evaluates specific ingredient contributions and their bio-functional effects on health and wellness. This process is crucial for understanding how these ingredients can prevent or combat diseases, allergens, aging impacts, and other ailments:
A. Nutritional Assessment Process: An RNS AI nutritional assessment is an opt-in, observed or reported comprehensive evaluation including both subjective and objective parameters and data to determine an individual, group, population or product nutritional impacts and status:
   a) Medical History and Dietary Intake: Collecting detailed information about past or current health objectives, issues and dietary habits; energy, protein and nutrient intake.
   b) Physical Issues: Opt-in report of physical signs of malnutrition; muscle wasting, edema.
   c) Dietary Baselines: Qualify & quantify food intake, types, frequency and caloric sources.
B. Influences of Aforementioned Artificial and Natural Ingredients:
   a) Artificial Ingredients: Synthetic food colors/additives commonly enhance appearance, taste, and shelf-life of food products, but they can have adverse health effects:
      i. Bio-functional Effects: Artificial ingredients alter gut microbiomes, affect digestion or lead to inflammation, impacting overall health negatively. RNS AI assesses this impact.
      ii. Health Risks: Synthetic colors and additives are linked to health issues like mutations, cancers & allergic reactions. Ultra-processed foods often contain artificial ingredients associated with risks of chronic diseases: cardiovascular disease and type 2 diabetes.
   b) Natural Ingredients: Foods and ingredients derived from wholefoods generally offer more health benefits compared to their artificial counterparts. Yet, RNS finds exceptions:
      i. Nutritional Value: Natural ingredients often provide essential nutrients, vitamins, and minerals that support bodily functions, health objectives and disease prevention.
      ii. Bio-functional Benefits: Many natural ingredients have anti-inflammatory, anti-oxidant, and immune-boosting properties that contribute to health and wellness. They can help mitigate or moderate effects of aging; reduce risk of chronic diseases.
C. Specific Contributions to Health and Wellness: Unique contributions of ingredients to health and wellness are assessed by examining food and nutrition roles and influences upon:
   a) Disease Prevention: RNS AI assesses both artificial and natural ingredient influences upon risks of developing diseases. Natural ingredients are often preferred for their protective effects, while artificial ingredients are more likely to be scrutinized for potential risks.
   b) Allergen Management: RNS AI identification of allergens in foods is crucial, especially when artificial ingredients or known exposures are present; can trigger allergic reactions.
   c) Aging & Ailments: Foods or natural ingredients rich in anti-inflammatory compounds or antioxidants may slow down aging processes or alleviate ailments whereas some artificial ingredients may exacerbate these issues. RNS AI analyzes & attributes key contributions.

(9) Relevant Nutrition Score (RNS and RNS AI): The present invention incorporates and leverages persistent and actionably applied bioscientific impact information (as described herein) integrated with global geo-demographic and culturally aligned crop sciences, food sciences, pharmaceutical information, environmental impacts, nutritional data, biological studies, scientific research, medical research, allergen databases, disease information, risk assessments, vitamins & minerals and metallurgy studies, and health & wellness knowledge (physical and mental), known or implied.

This information is dynamically integrated within algorithms that combine and weight relevant health objective(s), or preventative, reflective or responsive outcomes, leveraging macronutrient, micronutrient, phytonutrient and nano-nutritional metrics, absorption and utility through bio-availability metrics, nutrient impacts and associative values into uniquely persistent, reliable, actionable & interactive Relevant Nutrition Score (RNS and RNS AI), enabling the integrated methods, systems, software applications and services known as IRX FOODSCORE.

Critically, RNS AI forms actionable and interactive information that governments, hospitals, physicians, caregivers, food manufacturers, retailers and consumers can reasonably rely upon in use to improve efficacies and efficiencies of purposeful, prospective and persistent lifestyle or life stage consumption of foods, proactively, episodically or situationally as needed. RNS information and attributes enable a hospital, medical provider, caregiver, physician, health insurer, pharmaceutical drug or food manufacturer, as well as retailers, governing agencies and consumers to assess and score health relevance, benefits, risks and impacts of existing, planned or speculative products, consumables, prescriptive plans/care protocols, or alternatively to develop optimized and purposeful future or forward-looking (re)formulations or recipes.

RNS AI dynamically, inherently and specifically quantifies and qualifies nutritional intensity and caloric density required to optimize target immunities, or to support general or specific objective, active, situational or impaired lifestyles, while optimally impacting specific resilience or treatments of known or targeted diseases, allergens, lifestyles or other physical or mental impairments. RNS outputs are generated in support of select micro or mass populations or products across diverse ages, life stages and cultures (objective or imposed), and for purposeful nutritional directives, proactive, prescriptive or responsive dietary plans, and integrated treatment protocols, all aligned to positively impact healthy sustainable or situational lifestyles, defined by/for any single person, select group or global population.

### (10) AI-enabled Software, Algorithms and Applications (Artificial Intelligence-Enabled):

RNS AI is implementing and deploying a persistent and dynamic process that leverages a portfolio of robust and advanced AI-enabled software, algorithms and applications which assess nutrition and optimize user-aligned, health-specific and applied nutritional insights. The AI-driven process leverages a series of proprietarily integrated, concurrent and sequential RNS AI algorithms aimed at improving user, individual or population health outcomes, also assessing and scoring current or prospective products and future food innovations, by associating relevancy of use or purpose. This enables dietary assessments pro- or re-actively for diverse public or private audiences.

### A. Framework for RNS Implementation:

a) AI-enabled Data Collection: RNS AI engages diverse AI tools to source relevant data.
   i. Source and Extract Diverse Datasets: Collect diverse and disparate data from various sources (research defined herein) providing comprehensive datasets for analyses.
   ii. Ensure Data Interoperability: RNS AI employs AI-enabled applications, systems and tools that integrate data from different formats and sources to create unified databases which are crucial for enabling AI algorithms to access or process data effectively.
b) AI-enabled Algorithm Development: RNS AI utilizes AI tools & technology foundations.
   i. Develop Proprietary Algorithms: RNS AI creates algorithms that analyze collected data to identify patterns and correlations related to relevant, purposeful nutrition and health outcomes; algorithms capable of handling complex datasets and providing actionable insights in support of personal, population, medical or other users.
   ii. Leverage Machine Learning (ML) and AI Technologies: Utilize ML models to generate predictive analytics to forecast health risks; recommend personalized nutrition plans.
c) AI-enabled Personalization & Customization: RNS-AI Apps create relevant value.
   i. Create Personalized Health Plans: Use RNS-AI tools to tailor nutrition and health recommendations based upon user-specific or product insights, objectives, situational needs, circumstances, preferences or genetic profiles. This involves scoring specific existing foods, defining prospective new foods, and customizing nutritional plans.
   ii. Dynamic Adjustment of Recommendations: RNS-AI applications that implement sustainable systems, which dynamically adapt recommendations in real-time based on new data inputs, such as changes in health status, lifestyle or stated objectives.
d) AI-enabled Applications Deployment: RNS-AI created custom applications and user-aligned access points.
   i. Developed User-Friendly Interfaces: Design RNS AI user-friendly applications and platforms that are accessible for personal, medical and business end-users, including individuals, healthcare providers, and nutritionists. These interfaces facilitate easy interactions with an RNS-AI system, providing clear insights, product scores and purposeful nutrition insights.
   ii. Deployed on Scalable Platforms: RNS AI engages AI applications with cloud-based solutions and edge computing for scalable deployment, ensuring AI-enabled systems can handle large volumes of disparate data, dynamic scoring, and diverse end-users.
e) AI-enabled Ethical & Regulatory Compliance: RNS AI deploys secure environments.
   i. Data Privacy & Security: RNS AI deploys robust security measures to protect sensitive health data and to comply with data protection regulations such as GDPR or HIPAA.
   ii. Address Ethical Concerns: Develop transparent RNS AI systems that minimize biases and ensure fairness in recommendations. Regular content audits and scoring validations will be conducted to maintain trust and to ensure reliability and consistency, globally.

### B. Continuous RNS AI Improvement and Feedback:

a) Incorporate IRX FOODSCORE User Feedback: Continuously gather feedback from users to improve the RNS AI system's accuracy and user experience. This can involve iterative updates to the AI-enabled algorithms, user filters, conditions, and user interfaces.
b) Monitor and Evaluate Outcomes: Regularly assess effectiveness of RNS AI-driven nutritional recommendations in achieving health objectives and for fighting or preventing diseases. RNS AI uses this data to refine and optimize algorithms to improve system performance.

RNS AI is effectively implementing multi-tiered AI-enabled applications that optimize purposeful food consumption, nutrition insights and scores, while supporting individual, group, population, brand or product health objectives and outcomes (proactively or reactively) across diverse global geo-demographics, cultures, diseases, health concerns, conditions, opportunities and objectives.

(11) Food Information (RNS AI sources global nutrition information; public & private): RNS AI-enabled applications are leveraging multiple approaches to comprehensively source and analyze global food and nutrition information:
a) RNS AI Nutritional Data Aggregation:
   i. RNS AI integrates nutrition data from public nutrition databases like USDA FoodData Central, EuroFIR, and national food composition databases worldwide.
   ii. RNS AI accesses commercial food product databases containing nutrition information for branded and packaged foods.
   iii. RNS AI scrapes nutrition facts panels and ingredient lists from food manufacturers and retailer websites.
   iv. RNS AI collects recipe and menu data from restaurant chains, fast foods companies, and food service providers.
   b) RNS Nutrition(al) Research Integration:
      i. RNS AI mines scientific literature databases: PubMed product and ingredient studies, Web of Science for nutrition studies, clinical trials, nutrition databases & repositories.
      ii. RNS AI analyzes consumer packaged goods, dietary databases, food sciences and nutrition journals for latest research on nutrient bioavailability, health impacts, etc.
      iii. RNS AI incorporates data from large-scale nutrition surveys and cohort studies for single ingredient, purposeful recipe, formulaic functions, processed and natural foods.
   c) RNS Advanced Analytics:
      i. RNS AI uses natural language applications and processing to extract nutrition information from unstructured text in research papers, reports, etc.
      ii. RNS AI applies machine learning (ML) algorithms to identify patterns/relationships in nutrient profiles across foods, by ingredients, sources, production and processing.
      iii. RNS AI develops predictive models to estimate missing nutrient values based on similar foods, which are updated and re-validated persistently.
   d) RNS Global Coverage:
      i. RNS AI aggregates region-specific food composition, provision, production, processing and preparation practices data to account for regional, country or cultural nutrition priorities, sources and differences in fortification, processing, etc.
      ii. RNS AI incorporates traditional & Indigenous foods data; communities and cultures.
      iii. RNS AI tracks and assesses global food supply chains and nutrient changes through transport and storage.
   e) RNS Continuous Updating:
      i. RNS AI automates data pipelines; regularly refresh information at primary sources.
      ii. RNS AI detects and flags potential data inconsistencies & outliers for human review.
      iii. RNS AI allows for user-generated content and feedback to supplement, verify and validate information.

This comprehensive food and nutrition(al) approach allows RNS AI systems to create comprehensive, up-to-date nutrition knowledge-bases, which persistently fuel RNS AI and end-user applications for personalized nutrition, public health initiatives, and food industry innovation. RNS and RNS AI leverage diverse data sources and ensures data quality, while accounting for regional, cultural and country-specific variations in nutrition, foods, diets, availability and preservation methods, practices and substitutions.

(12) Food Preparation(al) Practices: RNS AI applications weigh impacts of food preparation. RNS AI-enabled applications revolutionize the way health impacts, nutrition bioavailability, real metabolization, functional nutrition and pharmaceutical efficacy, and applied understandings of how types of food preparation and cooking practices uniquely impact nutrition; leveraging vast amounts of global source data, nutrition panels, and research studies.

### A. Data Sourcing and Integration:

a) Global Databases: RNS AI-enabled applications access both private and public databases containing comprehensive information on food preparation methods, nutritional content, and known health impacts, including diverse and disparate databases maintained by global health organizations, culinary institutions, food manufacturers and food or product distributors.
b) Nutrition Panels and Studies: RNS AI-enabled applications integrate data published from nutrition panels and scientific studies, which provide insights into the nutritional content of various foods and how different cooking methods affect their nutrient profiles.
c) Research Databases: RNS AI-enabled applications analyze research papers and studies from global research databases to assess food preparation practices and corresponding nutritional impacts, benefits, and detriments of foods in different regions or countries.
B. Food Preparation Practices and Cooking Methods: Cooking methods impact nutritional values of any foods, affecting both retention and loss of vitamins & minerals, while potentially adding detrimental elements: chemicals, fats, oils, salts, charcoal and carbon exposures. RNS AI-enabled applications objectively and subjectively categorize processing and cooking practices, and consider detailed data and information from various regions, countries and cultures related to cooking and serving methods, dynamically attributing nutritional, health and wellness impacts upon users.
   a) RNS AI sources global data and research related to all types of ingredients, foods, preparation and cooking methods, and distinct influences and impacts upon nutrition, vitamins and minerals.
   b) RNS AI looks at all preparation practices and cooking types: e.g., ten prevalent cooking types listed below with basic impact observation:
      i. Raw: RNS AI observes that consuming some types of foods in their natural or raw states preserves the most nutrients. However, there may also be other consequences to health, digestibility or realized benefits, associated with culturally-aligned dietary practices.
      ii. Raw Food Nutrient Retention: Eating raw foods preserves all nutrients, vitamins and minerals since there is no heat involved that could cause nutrient degradation.
      iii. Raw Food Bioavailability: Some nutrients are more bioavailable when certain foods are cooked, as cooking may break down a raw food's cell walls, unlocking nutrients, making it easier for a body to absorb. However, raw consumption maintains highest levels of water-soluble elements like vitamin C and B vitamins.
      iv. Boiling and Steaming: RNS AI observes that boiling foods may cause nutrient loss in water (temperature dependent); steaming generally retains more nutrients.
         a. Boiling Nutrient Loss: Preparation practices such as boiling are known to effect significant losses of water-soluble vitamins such as vitamin C and B vitamins, as these nutrients leach into the water. 50% or more of these vitamins can be lost during boiling.
         b. Boiling Mineral Retention: Boiling foods results in minerals that are generally more stable; some loss of key minerals occurs if cooking water not consumed.
         c. Steaming Nutrient Retention: Steaming is one of the best methods for preserving a food's nutrients, especially water-soluble vitamins and minerals. Steaming minimizes nutrient loss by avoiding direct contact with water.
         d. Steaming Flavor and Texture: Steaming is excellent for nutrient retention, but may result in less flavorful dishes, unless seasonings & spices are used, which may be used proactively for beneficial health objectives and limited diets.
      v. Frying and Air Frying: RNS AI observes that the practice of frying foods (regardless of oils) alters nutritional profiles by adding unhealthy fats, whereas Air Frying often uses less oil, and likely retains more nutrition. However, practices differ by foods, culture, and country.
         a. Frying Nutrient Loss: Frying often results in higher fat content and can degrade certain nutrients due to high temperatures. However, it can also increase the bioavailability of some fat-soluble vitamins.
         b. Frying Flavor Enhancement: Many cultures embrace fried foods; frying often enhances flavors and textures of foods, making them more palatable and tastier. And many people eat fried foods globally, unaware of potentials for detrimental health effects.
         c. Air Frying Nutrient Retention: Air frying uses less oil at higher temperatures for shorter time periods, which helps retain nutrients, especially as compared to traditional frying methods. Air frying actually enhances antioxidant properties in certain vegetables.
         d. Air Frying is Healthier Alternative: Air frying is considered a healthier option than deep frying, as it reduces unnecessary fat content while still maintaining a crispy texture.
      vi. Grilling and Roasting: RNS AI observes this cooking method enhances flavors; may result in nutrient loss; may form harmful compounds if not done properly.
         a. Grilling Nutrient Loss: Grilling often leads to loss of B vitamins and minerals, due to dripping juices in meats, fish or poultry, losing up to 40% of nutrients.
         b. Grilling Health Concerns: Grilling may potentially produce some harmful compounds like polycyclic aromatic hydrocarbons (PAHs); created when fats drip onto heat source.
         c. Roasting Nutrient Retention: Roasting generally minimizes losses of vitamin C compared to other methods (e.g., boiling); uses less water and often shorter cooking times. However, some nutrient loss still occurs due to heat involved.
         d. Roasting Flavor & Texture: Roasting can enhance flavors by caramelizing natural sugars in foods. Deeper, richer, more concentrated tastes appeal to some cultures more than others.
      vii. Baking and Toasting: RNS AI applies nutritional impact of baking or toasting.
         a. Baking Nutrient Loss: Baking can lead to destruction of certain nutrients, particularly in the crust of baked goods, due to high temperatures. Vitamin C and B complex vitamins are often reduced during a baking process.
         b. Baking Benefits: Despite some nutrient loss, baking is good for preserving proteins & some nutrients in meats, poultry & fish; grains are easier to digest.
         c. Toasting Nutrient Changes: Toasting bread can lead to a decrease in certain nutrients, such as thiamine (vitamin B1) and lysine, an essential amino acid. However, the overall nutritional changes are minimal unless bread is burnt.
         d. Toasting Glycemic Index: Toasting can lower glycemic index of bread, making a better option for people with diabetes; Causes slower release of glucose into bloodstream.
         e. Toasting Acrylamide Formation: The browning process during toasting can lead to the formation of acrylamide, suspected as carcinogenic especially if bread is over-toasted.
      viii. Barbecuing: RNS AI observes that there is a need to cook with this method; primary or secondary (e.g., no other available sources).
         a. Barbeque Nutrient Loss: Barbecuing is cooking over open flame, which can result in formation of potentially harmful compounds like polycyclic aromatic hydrocarbons (PAHs). A nutrient loss, particularly of B vitamins, can occur due to dripping juices.
         b. Barbeque Flavor & Risks: Barbecuing may enhance flavors through known reactions, but it may increase risks of cancer with formation of certain detrimental compounds.
      ix. Microwaving: RNS AI values global needs of available microwave cooking.
         a. Microwaving Nutrient Retention: Microwaving is one of the best methods for retaining nutrients, especially in vegetables, due to short cooking times and minimal water use, and it preserves most vitamins and minerals, as well as antioxidants in some foods.
         b. Microwaving Nutrient Loss: Microwaving may create vitamin C loss, yet it is generally less loss than observed with other cooking methods listed above.

### C. Diverse Nutritional Impacts and Bioavailability:

a) Nutrient Retention: RNS AI recognizes varied cooking methods do affect nutrient retention, densities and bio-availabilities differently, such as steaming and microwaving, which tend to preserve vitamins vs. boiling. This is important and specifically speaks to like-food scoring variances, when prepared and consumed by different cooking methods.
b) Bioavailability: RNS AI has found that certain types of cooking can increase or decrease the bioavailability of certain nutrients, minerals and phytochemicals, making them easier or more difficult to digest and absorb, especially when combatting illness. As an example, cooking tomatoes increases the availability of lycopene, a beneficial phytochemical.
c) Impact of Processing: RNS AI applications dynamically assess how food preparation and processing affects nutritional content. For instance, ultra-processed foods often have reduced nutritional values and contribute to health issues like obesity and cardiovascular diseases, and are exacerbated by certain types of cooking and preparation methods.
d) Water-Soluble Vitamins: RNS AI distinguishes between cooking types involving water, like boiling, which can lead to significant losses of water-soluble vitamins, such as vitamin C and B vitamins. Whereas, steaming retains more nutrients; less water dilution.
e) Fat-Soluble Vitamins: RNS AI research shows key vitamins are more stable during cooking; methods involving high heat or prolonged cooking times cause some loss.
f) Mineral Retention: RNS AI observes that some minerals tend to be more stable than vitamins which vary by cooking process; can still be lost when food juices are discarded.

### D. RNS AI Applications in Practice:

a) Personalized Cooking Guidance: RNS AI tools and applications can provide personal cooking suggestions based on type of food and desired nutritional outcomes. RNS AI can suggest optimal air frying settings to preserve nutrients; ensuring food efficacy.
b) Nutritional Analysis: RNS AI tools and applications can offer real-time nutritional analyses of nutrients, foods and meals based upon ingredients and cooking methods used, helping users make informed or purposeful nutrition, foods and proactive dietary choices.

RNS AI applications acknowledge diverse cooking methods have their own distinct impacts on nutritional content of foods, and while cooking can improve digestibility and bioavailability of foods and certain nutrients, it can also lead to the loss of others, particularly water-soluble vitamins. The choice of cooking method should balance nutrient retention with flavor and texture preferences. Methods like steaming and air frying generally preserve more nutrients, while methods like boiling and frying result in greater nutrient loss. Toasting, specifically, has minimal impact on nutrition but can alter glycemic index or increase acrylamide formation if overdone.

In summary, RNS AI-enabled applications will transform many proactive and purposeful practices of food preparation, integrating diverse data sources to provide comprehensive insights into cooking methods and nutritional impacts. This creates more informed choices that enhance health and well-being, fighting diseases naturally across different peoples, regions and cultures.

(13) Additives: RNS AI applications capture additives and preservatives (Artificial or Natural). Below, RNS AI provides a global example list (not exhaustive) of currently known artificial and natural additives along with a list of artificial and natural preservatives used in food products (dynamically evolving over time). RNS AI applications consider these broadly used ingredients in determining purposeful properties or impacts to target users, people, populations and products.

### A. Artificial Additives: (Sweetener, Color, Flavor, Emulsifier, Stabilizer, Thickener, Anti-Cake)

a) Artificial Sweeteners:
   i. FDA-Approved Artificial Sweeteners
      a. Aspartame (NutraSweet, Equal), Acesulfame potassium (Ace-K, Sunett, Sweet One), Sucralose (Splenda), Saccharin (Sweet' N Low, Sugar Twin), Neotame (Newtame), Advantame. (Sources updated by country as identified or approved)
b) Other Artificial Sweeteners
   i. Alitame (approved in some countries, not in the US), Sodium cyclamate (banned in the US, approved in many other countries), Salt of aspartame-acesulfame, Neohesperidin dihydrochalcone.
   c) Unique Sweeteners (Often considered artificial)
      i. Steviol glycosides (Stevia extracts), Monk fruit extract
   d) Sugar Alcohols (Sometimes classified as artificial sweeteners)
      i. Erythritol, Xylitol, Sorbitol, Maltitol, Isomalt, Lactitol
   e) Other Sweeteners
      i. Allulose (considered a rare sugar; sometimes grouped with artificial sweeteners)
      ii. Tagatose

In summary, RNS AI observes that artificial sweeteners vary in nutritional characteristics (beneficial or detrimental), sweetness intensity, caloric content and regulatory approval across different uses and countries. Regardless of FDA or other agencies, research on long-term effects and impacts of sweeteners is ongoing, and relevant recommendations may evolve as new evidence emerges.

### B. Artificial Additives: (Sweetener, Color, Flavor, Emulsifier, Stabilizer, Thickener, Anti-Cake)

a) FDA-Approved Artificial Food Colors (Including, not limited to; Under FDA Review)
   i. Red 40 (Allura Red AC)
      a. Most widely used food dye; May cause allergy-like reactions and hyperactivity in some children; Potential carcinogen, though evidence is inconclusive
   ii. Red 3 (Erythrosine)
      a. Linked to thyroid tumors in animal studies; May cause hypersensitivity reactions; Use has been reduced but still approved for some foods
   iii. Yellow 5 (Tartrazine)
      a. Second most common food dye; May cause allergy-like reactions, especially in aspirin-sensitive individuals; Linked to hyperactivity in some children
   iii. Yellow 6 (Sunset Yellow FCF)
      a. Third most common food dye; May cause allergy-like reactions; exacerbated with age or allergens; Potential carcinogen (evidence remains inconclusive)
   iv. Blue 1 (Brilliant Blue FCF)
      a. May cause allergic reactions; range of effects dependent upon other health; Some evidence of potential neurotoxicity, but more research needed
   v. Blue 2 (Indigotine)
      a. Evidence of potential brain cancer risk in animal studies, but FDA supports that it remains safe for consumption. RNS monitoring PubMed research studies.
   vi. Green 3 (Fast Green FCF)
      a. Rarely used; Evidence of bladder and testes tumors in animal studies; FDA supports that it remains safe for consumption. RNS monitoring PubMed research studies.
   vii. Citrus Red 2
      a. Only approved for coloring orange peels; potentially harmful if concentrated; Potential carcinogen at high doses, but risk from normal consumption is low
b) RNS AI observes, and researches health concerns and impacts of Artificial Colors:
   i. Hyperactivity and behavioral issues in children
   ii. Allergic reactions and hypersensitivity
   iii. Potential carcinogenic effects (though evidence is often inconclusive)
   iv. Possible neurotoxicity
   v. Thyroid issues (specifically linked to Red 3)
   vi. Potential organ damage at very high doses (rarely relevant to normal consumption)
   c) RNS AI general considerations of Artificial Colors:
      i. Individual sensitivity varies greatly
      ii. Most studies showing serious health effects used extremely high doses
      iii. Natural alternatives may also cause reactions in sensitive individuals
      iv. FDA considers approved dyes safe at current usage levels (disputed)
      v. Some countries require warning labels for certain dyes (given observations)
      vi. Avoiding artificially colored foods generally means eating less processed food, which has other health benefits address by RNS AI applications.

In summary, RNS AI applications persistently evaluate global research/clinical studies to determine real detrimental issues and attributes of artificial colors, as the US Food and Drug Administration (FDA) and other global regulatory bodies consider most dyes “safe” at levels typically consumed. However, some health organizations and consumer groups advocate further research and stricter regulations given impacted populations.

### C. Artificial Additives: (Sweetener, Color, Flavor, Emulsifier, Stabilizer, Thickener, Anti-Cake)

a) Artificial Flavor Enhancers:
   i. Glutamates
      a. Monosodium Glutamate (MSG)
         i) May cause headaches, nausea, and other symptoms in sensitive individuals (known as MSG symptom complex)
         ii) Potential link to obesity and metabolic disorders; evidence is inconclusive
         iii) Generally recognized as safe (GRAS) by the FDA
      b. Other Glutamates (Monopotassium glutamate (MSG), Calcium diglutamate, etc.) Similar potential effects as MSG, but less commonly used and studied
      RNS AI recognizes potential MSG Health Concerns: MSG symptoms and side-effects are complex and may serve to exacerbate existing issues or to create new conditions in sensitive individuals:
         i) Headaches, Flushing, Sweating, Nausea, Numbness, Tingling, Palpitations, Drowsiness, Metabolic effects:
         ii) Potential link to obesity and metabolic disorders; evidence is evolving
         iii) Neurotoxicity: Studies suggest potential neurotoxic effect; research ongoing
         iv) Reproductive effects: Studies show potential impacts to reproductive organs
         v) Cardiovascular effects: Studies report changes in blood pressure & heart rate
   ii. Nucleotides
      a. Disodium Guanylate (E627) and Disodium Inosinate (E631)
         i) Generally considered safe
         ii) May cause gout flare-ups in susceptible individuals due to purine content
         iii) Often used in combination with MSG for synergistic effect
      b. Hydrolyzed Vegetable Protein (HVP)
         i) Contains naturally occurring MSG
         ii) May cause similar reactions as MSG in sensitive individuals
         iii) Potential allergen concerns for those with soy or wheat allergies
      c. Maltol (E636) and Ethyl Maltol (E637)
         i) Generally recognized as safe
         ii) Evidence of potential genotoxicity at very high doses in animal studies

RNS AI applications capture impacts from studies and historical reports wherein individuals, groups, regions or populations demonstrate high sensitivities to one or more artificial flavor enhancers, and most studies showing serious health effects used extremely high doses.

However, RNS AI observes that avoiding artificially flavored foods generally means eating less processed food, which often has other health benefits, especially when fighting diverse disease states.

RNS AI applications and research also recognize direct correlations with mass introductions of convenience related products into new retail channels, areas, regions or countries. For instance, studies showing growth of access to cheap frozen foods or fast take-out foods generally reflect higher levels of MSG, sodium and many artificial ingredients and preservatives impacting health.

### D. Artificial Additives: (Sweetener, Color, Flavor, Emulsifier, Stabilizer, Thickener, Anti-Cake)

a) Artificial Emulsifiers:
   i. Lecithin (E322):
      a. Purpose: Acts as an emulsifier to help mix ingredients like oil and water.
      b. Health Impacts: RNS AI research defines lecithin as generally safe, but some studies suggest potential impacts on gut microbiota without significant adverse effects.
   ii. Polysorbate 80 (E433):
      a. Purpose: Used to stabilize and emulsify foods.
      b. Health Impacts: RNS AI data suggests polysorbate 80 causes bacterial translocation and intestinal inflammation, potentially contributes to metabolic syndrome.
   iii. Carboxymethylcellulose (E466):
      a. Purpose: Serves as a thickener and stabilizer.
      b. Health Impacts: RNS AI measures gut microbiota impact; inflammatory response.
   iv. Mono- and Diglycerides:
      a. Purpose: Serves to emulsify and stabilize foods.
      b. Health Impacts: RNS AI observes that glycerides are generally recognized as safe, but excessive intake may contribute to obesity and metabolic disorders.

### E. Artificial Additives: (Sweetener, Color, Flavor, Emulsifier, Stabilizer, Thickener, Anti-Cake)

a) Artificial Stabilizers and Thickeners:
   i. Carrageenan (E407)
      a. Purpose: Used to thicken and stabilize foods like dairy products and desserts.
      b. Health Impacts: RNS AI research shows E407 may cause intestinal inflammation and alter gut microbiota, raising concerns about inflammatory bowel diseases.
   ii. Guar Gum (E412)
      a. Purpose: Acts as a thickener and stabilizer in various foods.
      b. Health Impacts: RNS AI sees E412 as generally safe, but excessive consumption can lead to digestive issues like gas and bloating.
   iii.Xanthan Gum (E415)
      a. Purpose: Used to thicken and stabilize foods.
      b. Health Impacts: RNS AI observes that E415 is generally safe; however may cause potential digestive discomfort if consumed in large amounts.
   iv. Agar (E406)
      a. Purpose: Functions as a gelling agent and thickener.
      b. Health Impacts: RNS AI research shows E406 has minimal adverse effects reported.

### F. Artificial Additives: (Sweetener, Color, Flavor, Emulsifier, Stabilizer, Thickener, Anti-Cake)

a) Artificial Anti-Caking Agents:
   i. Silicon Dioxide (E551)
      a. Purpose: Prevents clumping in powdered foods.
      b. Health Impacts: RNS AI recognized as safe, with no significant health concerns at typical consumption levels; Exceptions in high amounts; Age/health dependent.
   ii. Sodium Aluminosilicate (E554)
      a. Purpose: Used to prevent caking effect and clumping in powdered products.
      b. Health Impacts: RNS AI research shows that excessive intake of E554 potentially affects kidney function, due to concentration of aluminum content.
   iii. Calcium Silicate (E552)
      a. Purpose: Prevents caking in powdered foods.
      b. Health Impacts: RNS AI research shows E552 mostly safe; no major health impacts.

RNS AI recognizes that artificial additives are widely used throughout global food industries in efforts to enhance texture, stability, and shelf life at lower prices. Despite being considered “generally safe” (e.g., US GRAS) by regulatory agencies, artificial additives have been directly linked to health concerns; specifically gut health and metabolic disorders. RNS AI makes it simple for all users to assess food-related risks associated with food additives and an understanding of related and user-relevant health impacts.

As an example, health impacts of artificial additives and ingredients in processed, preserved, and fast foods have been extensively studied across different populations, age groups, and disease states. RNS AI integrates and leverages this global research and clinical studies to distinguish impacts across geo-demographic groups; potential for detrimental or exacerbated effects based upon these findings differ by user-aligned target populations and end consumers:

### G. Artificial Additives; Health Impact Examples by Age and Population:

a) Children:
   i. ADHD and Behavioral Issues: Studies sourced by RNS AI suggest links between artificial food colorings and hyperactivity in children, but evidence is mixed.
   ii. Obesity: RNS AI observes that children are more susceptible to obesogenic effects of food additives due to age, weight, higher relative intake compared to adults.
b) Adults:
   i. Cardiovascular Diseases (CVD): RNS AI research shows that higher consumption of ultra-processed foods, which often contain artificial additives, is associated with an increased risk of varied cardiovascular diseases.
   ii. Metabolic Disorders: RNS AI research shows emulsifiers, and other artificial additives contribute to metabolic syndrome; altered gut microbiota, promoting inflammation.
c) Pregnant Women and Infants:
   i. Developmental Concerns: Some additives may interfere with fetal development, and there is a call for caution in their consumption during pregnancy.
   ii. Infant Health: World Health Organization recognizes 60% of baby foods fail basic nutrition: e.g., 70% failed protein; 44% too much sugar; 20% too much salt.
   iii. Health Impacts: RNS AI observes poor infant/child nutrition early in life promotes chronic diseases: eating disorders, obesity, diabetes, some cancers later in life.

### H. Artificial Additives; Health Impacts—Examples by Disease State:

a) Cardiovascular Health:
   i. Increased CVD Risk: RNS AI recognizes that ultra-processed foods are often linked to higher risks of CVD; poor nutrient profiles and the presence of harmful additives.
   ii. Hypertension and Dyslipidemia: RNS AI acknowledges artificial additives directly influence blood pressure and lipid profiles, contributing to cardiovascular risks.
b) Cancer:
   i. Potential Carcinogens: RNS AI sees some artificial additives and contaminants formed by food processing exhibit carcinogenic properties, increasing cancer risk.
c) Metabolic Health:
   i. Type 2 Diabetes & Obesity: RNS AI data shows artificial additives in ultra-processed foods contribute to insulin resistance and obesity; cause gut microbiota alterations.
   ii. Inflammation: RNS AI analyzes that certain artificial emulsifiers and preservatives can promote inflammation, exacerbating conditions like metabolic syndrome.
d) Gut Health:
   i. Microbiota Dysbiosis: RNS AI research shows that artificial additives like emulsifiers can disrupt gut microbiota, leading to digestive issues and potentially contributing to inflammatory bowel diseases.

Overall, while many artificial additives are considered theoretically safe when consumed at so-called regulated levels, their cumulative effects, especially as used with ultra-processed foods, show direct long-term health impacts. RNS AI captures and reflects this body of evidence within its scores, grades and risks, and in a relevant manner, given other user-defined attributes and filters. Diverse data sources and a body of global evidence and regulations show that these effects can vary based on individual susceptibility, age, predisposed or pre-existing health conditions.

In summary, artificial food additives have come under increasing scrutiny globally (e.g., fast foods, snacks, cereals and beverages, etc.) due to their studied negative impacts on human health, particularly in children. Critically, studies have linked these synthetic chemicals to a range of concerning effects, including neurotoxicity, cytotoxicity, genotoxicity, and carcinogenicity. Some additives have also been associated with behavioral issues in children, exacerbating symptoms of attention deficit hyperactivity disorder (ADHD). And long-term consumption of ultra-processed foods containing multiple additives may contribute to increased risks of obesity, cardiovascular disease, metabolic syndrome, and certain cancers. Artificial sweeteners, in particular, have been linked to an elevated risk of cancer and cardiovascular diseases. Moreover, certain additives like artificial food colors, preservatives, and emulsifiers may interfere with hormonal balance, while potentially affecting growth and development. Given these concerns, there is a growing global call for stricter regulations of food additives, particularly those used in products marketed to children, and a shift towards natural, unprocessed foods to promote better health outcomes, all of which can be measured and risk assessed through RNS scores and user-aligned criteria.

(14) Artificial Additives & Preservatives Types, Health Impacts and Methods (Natural Ahead): RNS AI recognizes that artificial preservatives are widely used throughout the world in the production of various food products, including processed, preserved, and fast foods. They serve to extend shelf life, prevent spoilage, and maintain the quality of food. However, these preservatives often have negative health impacts, which vary across different populations and disease states. RNS evaluates types and impacts of preservation, types of artificial preservatives.

RNS AI monitors a minimum of seven common types (more uncommon and emerging) of artificial preservatives (chemical preservation methods) and their specific health impacts by type, but also recognizes that there are other less used artificial preservatives and methods within specific countries and cultures, dependent upon food processing practices by types and purpose:

### A. Artificial Preservatives: 7 Common Types Artificial Preservatives; Example Health Impacts

a) Nitrates and Nitrites
   i. Purpose: Used in processed meats to prevent bacterial growth and enhance color.
   ii. Health Impacts: RNS AI captures links to increased risks of cancer, particularly colorectal cancer, due to the formation of nitrosamines (carcinogens) within the body.
b) Benzoates (e.g., Sodium Benzoate)
   i. Purpose: Used in acidic foods: e.g., fruit juices, soft drinks, pickles; limit microbials.
   ii. Health Impacts: RNS AI observes allergic reactions and asthma in sensitive individuals; concerns about carcinogenic effects when combined with ascorbic acids (vitamin C).
c) Sulfites (e.g., Sulfur Dioxide)
   i. Purpose: Uses; preserve color, prevent spoilage (dried fruits, wines, processed foods).
   ii. Health Impacts: RNS AI studies show triggering of asthma attacks and allergic reactions in sensitive individuals. Long-term exposures may lead to other respiratory issues.
d) Sorbates (e.g., Potassium Sorbate)
   i. Purpose: Inhibits mold and yeast in foods like cheese, baked goods, and beverages.
   ii. Health Impacts: RNS AI recognizes as generally safe; may cause skin irritation and other allergic reactions and irritations in some people.
e) Parabens
   i. Purpose: Use—Beverages, baked goods, processed meats; prevents microbial growth.
   ii. Health Impacts: RNS AI captures endocrine disruptors, with concerns about their role in hormone-related cancers.
f) Butylated Hydroxyanisole (BHA) and Butylated Hydroxytoluene (BHT)
   i. Purpose: Antioxidants used to prevent fats and oils from becoming rancid in foods like cereals, snacks, and chewing gum.
   ii. Health Impacts: RNS AI monitors possible carcinogens; studies suggest links to cancer and endocrine disruption.
g) Propionates (e.g., Calcium Propionate);
   i. Purpose: Used in baked goods to inhibit mold growth.
   ii. Health Impacts: RNS AI sees that studies show this as generally safe. However, frequent or high consumption may lead to headaches and other progressive digestive issues.

### B. Artificial Preservatives; Example Health Impacts by Population and Disease State:

a) Children:
   i. RNS AI research studies show vulnerability to hyperactivity and behavioral issues due to preservatives like artificial colors and sodium benzoate; also risk asthma & allergies.
b) Adults:
   i. RNS AI documents increased risks of cancer, cardiovascular diseases, and metabolic disorders due to long-term consumption of preservatives: nitrates, nitrites, BHA/BHT.
c) Asthmatics and Allergy-Prone Individuals:
   i. RNS AI shows likelihood of adverse reactions such as allergic reactions, asthma attacks, and other adverse or exacerbated health issue impacts by sulfites and benzoates.
d) Pregnant Women:
   i. RNS AI reflects varied developmental concerns caused from frequent use and prolonged exposure to certain artificial preservatives, necessitating caution during pregnancy.

Overall, while artificial preservatives play crucial roles in food preservation, potential health risks necessitate careful RNS AI incorporation, interpretation and application when consumed by user-populations or when utilized within target products or brands. Moderation and limitation is encouraged when practical. Natural alternatives should be considered when practical or possible.

C. Artificial Preservatives; 3 Types of Preservation Methods (Chemical, Physical, Advanced): Food preservation is a crucial practice that extends the shelf life of food, prevents spoilage, and maintains quality, taste and nutritional values, while unfortunately also introducing adverse health impacts and side-effects. Various artificial preservation methods are used globally by different countries and cultures, each with its own unique processes and impacts. Here is a demonstrable list of food preservation types/practices for artificially preserving foods:
a) Chemical Preservation Methods: (Updated research may change over time)
   i. As previously listed under Artificial Preservatives; Example list of six chemical preservation methods considered by RNS AI applications in our scoring algorithms:
      a. Benzoates (e.g., Sodium Benzoate): Inhibit microbial growth in acidic foods;
      b. Nitrites and Nitrates are used in cured meats to prevent bacterial growth
      c. Sulfites (e.g., Sulfur Dioxide) are used to preserve color and prevent spoilage
      d. Sorbates (e.g., Potassium Sorbate) are used to inhibit mold and yeast in foods
      e. Parabens used in beverages, baked goods, processed meats; prevent microbials
      f. BHA (Butylated Hydroxyanisole) and BHT (Butylated Hydroxytoluene) are chemical antioxidants; prevent fats/oils from becoming rancid: foods, cereals, snacks.
b) Physical Preservation Methods: (Updated research may change over time)
   i. Irradiation:
      a. Purpose: Ionizing radiation; kills bacteria & parasites in foods, spices and meats.
      b. Health Impacts: RNS AI values as generally safe; However, potential nutrient loss.
   ii. High-Pressure Processing:
      a. Purpose: Uses high pressure to inactivate pathogens and enzymes without heat.
      b. Health Impacts: RNS AI sees as maintaining nutritional quality; generally safe.
   iii. Ultrasound and Ozone Treatment:
      a. Purpose: Used to preserve heat-sensitive foods by reducing microbial load.
      b. Health Impacts: RNS AI sees as generally safe; effective to enhance food safety.
   c) Advanced Preservation Techniques: (Updated research may change over time)
      i. Nanotechnology:
         a. Purpose: Utilizes nanoparticles to enhance food preservation and packaging.
         b. Health Impacts: Emerging technology; ongoing research into safety and efficacy.
   ii. Modified Atmosphere Packaging:
      a. Purpose: Alters the atmosphere inside packaging; used to extend shelf life.
      b. Health Impacts: RNS AI observes as generally safe; widely used in the food industry.

In summary, RNS AI persistently evaluates artificial preservation methods, which are integral to modem food production, allowing for longer shelf life and reduced food waste.

However, some chemical preservatives have raised health concerns, particularly regarding allergies, asthma, and carcinogenic effects. RNS AI balances benefits or availability of preservation with potential or realized health risks. Ongoing research continues to explore safer and more natural alternatives.

(14) Natural Additives & Preservatives (Examples Only; Not All Inclusive): Natural ingredients are also widely used in the production of foods, consumables, fast foods, and preserved foods to enhance flavor, color, texture and shelf-life while maintaining a “clean” label. Below are RNS AI examples of natural additives; types, purposes, or objective health impacts:

A. Natural Sweeteners: Natural sweeteners are used as additive alternatives to refined sugar, offering various benefits for health, as natural ingredients. Here is a list of common “natural” sweeteners, purposes in food, and key sample health impacts weighted by RNS AI:
a) Raw Honey
   i. Purpose: Used as a sweetener in beverages, baked goods, bars and sauces.
   ii. Health Impacts: RNS AI recognizes honey contains antioxidants, vitamins, and minerals; exhibits antimicrobial properties and soothes sore throats. Honey is high (dense) in calories. Potential weight gain (good/bad) if excess consumed.
b) Stevia
   i. Purpose: Natural zero-calorie sweetener used in beverages, desserts, snacks.
   ii. Health Impacts: RNS AI captures Stevia's low glycemic index; suitable for people with diabetes; does not spike blood sugar levels. Has bitter aftertaste.
c) Agave Nectar
   i. Purpose: Used in beverages, desserts, and as a sugar substitute in recipes.
   ii. Health Impacts: RNS AI sees lower glycemic index than sugar, but high in fructose; increases triglycerides or lead to fatty liver disease in large amounts.
d) Maple Syrup
   i. Purpose: Used in pancakes, waffles, desserts; natural sweetener in recipes.
   ii. Health Impacts: RNS AI tracks antioxidants & minerals like zinc and manganese. It is still high in sugar and should be consumed in moderation. Dense calories.
e) Coconut Sugar
   i. Purpose: Used as a substitute for brown sugar in baking and cooking.
   ii. Health Impacts: RNS AI tracks trace amounts of vitamins and minerals. It has a lower glycemic index than regular sugar, but is still high (dense) in calories.
f) Date Palm Sugar
   i. Purpose: Used in baking, smoothies and as a natural sweetener in energy bars.
   ii. Health Impacts: RNS AI measures dates as high in fiber, potassium & antioxidants. Dates help with digestion; calorie-dense and should be consumed in moderation.
g) Molasses
   i. Purpose: Used in baking, sauces, and as a sweetener in beverages.
   ii. Health Impacts: RNS AI research validates rich in iron, calcium, and magnesium. It provides nutritional benefits. but is high in natural sugar content.
h) Monk Fruit Extract
   i. Purpose: A zero-calorie sweetener used in beverages and low-calorie snacks.
   ii. Health Impacts: Does not affect blood sugar levels; suitable for diabetics. RNS AI generally considers as safe with no known adverse effects. Ongoing studies.
i) Erythritol
   i. Purpose: Used in sugar-free candies, baked goods, and beverages.
   ii. Health Impacts: RNS AI acknowledges low in calories and does not raise blood sugar levels. May cause digestive discomfort/cramps in large amounts.

In summary and when considering general health and risks of natural sweeteners, RNS AI observes both dietary benefits and cautions:
a. Benefits: Natural sweeteners often contain additional nutrients and have a lower impact on blood sugar levels compared to refined sugar.
b. Cautions: Despite being natural, these sweeteners can still contribute to excessive calorie intake if not consumed in moderation. Some, like agave nectar, have high fructose content, which can have negative health effects if consumed excessively.

Overall, natural sweeteners are a popular choice for those looking to reduce refined sugar intake while still enjoying sweet flavors. However, RNS AI exposes users to a need to be mindful and aware of types, qualities and quantities consumed; seek health-aligned and dietary goal options.

### B. Natural Colors: Plant-Based Colors (Contain Health Beneficial Phyto/Nano-Nutrients)

a) Red/Pink Shades:
   i. Beetroot extract, Anthocyanins from berries and other fruits, Lycopene from tomatoes, Red cabbage extract, Paprika extract
b) Orange/Yellow Shades:
   i. Carotenoids (beta-carotene, lutein, zeaxanthin), Annatto extract, Turmeric and curcumin, Saffron, Carrot extract
c) Green Shades:
   i. Chlorophyll, Spirulina extract, Matcha powder, Spinach powder
d) Blue/Purple Shades:
   i. Spirulina extract, Butterfly pea flower extract, Purple sweet potato extract, Grape skin extract
e) Brown Shades:
   i. Caramel (from heated sugar), Cocoa powder
f) Animal-Derived Colors
   i. Carmine (from cochineal insects), Squid ink (for black color)
g) Mineral-Based Colors
   i. Iron oxides, Titanium dioxide (white)
h) Other Natural Sources
   i. Activated charcoal (black), Huito fruit extract (blue)

RNS AI recognizes that natural food colors are derived from edible sources like fruits, vegetables, spices, flowers and other plants, and often contain many beneficial nutrients. They are used globally in foods to enhance the visual appeal of foods and beverages, while meeting consumer demand for health-positioned and “clean label” products. Specific natural colors used vary by region based upon local regulations, ingredient availability, consumer taste, source quality and health objectives.

C. Natural Flavors: RNS AI differentiates between the function and nutrition associated with natural flavors, which are typically used in small quantities and do not contribute significantly to caloric or nutrient content of foods. They are primarily added to enhance taste or aroma rather than for nutritional purposes.

Overall, RNS AI recognizes that while natural flavors may not directly impact nutrition, they are not necessarily healthier than artificial flavors. RNS supports a user's ability to recognize that both types of flavors undergo processing and may contain additional chemicals or preservatives.

RNS AI observes that natural flavors are generally considered safe for consumption, however research shows that many may contain chemicals and additives. The long-term health effects of many natural flavors are not fully understood, as individual reactions may vary. People with allergies or special dietary needs should be cautious and aware of the specific components and sources of any natural flavors claimed or present in their foods. User-aligned RNS AI scores and insights are created to help expose health concerns or to evaluate certain ingredients, foods and known sources, also denoting and known risks and benefits, if known or studied.

Below, RNS AI has identified a sample list of thirty commonly used naturalflavors, and their associated studied simple risks or benefits, their “natural” source, as well as any know additives or preservatives used with the named naturalflavor. RNS AI provides actionable transparency to both risks & benefits, which is not normally available to an average user; undisclosed in foods.
a) Diacetyl (butter flavor)
   i. Risk: Associated popcorn lung; abnormal lung function & shortness of breath
   ii. Benefit: No known benefits
   iii. Source: Naturally occurring in butter, beer, and some fruits
   iv. Common additive: Often accompanied by acetoin or acetyl propionyl
b) Monosodium Glutamate (MSG)
   i. Risk: Linked to obesity, metabolic disorders, and potential neurotoxic effects
   ii. Benefit: Can reduce overall sodium intake by 20-40% when combined with salt
   iii. Source: Derived from fermented sugar beets, sugar cane, or molasses
   iv. Common additive: Often accompanied by disodium inosinate, disodium guanylate
c) Citral (lemon flavor)
   i. Risk: Skin/mucous membrane irritant; potential reproductive toxicity concerns
   ii. Benefit: Reduce obesity, boost insulin sensitivity, may improve glucose tolerance
   iii. Source: Found in lemongrass, lemon, orange, and pimento
   iv. Common additive: Often stabilized with antioxidants like BHT or BHA
d) Methyl N-Acetyl Anthranilate (berry flavor)
   i. Risk: May cause user phototoxicity leading to skin irritation with exposure to sun
   ii. Benefit: No known benefits
   iii. Source: Derived from grapes and other berries
   iv. Common additive: Often accompanied by propylene glycol as a solvent
e) Castoreum (vanilla-raspberry flavor)
   i. Risk: Derived from beaver anal secretions; may be unpalatable to consumers
   ii. Benefit: No known health risks in low amounts
   iii. Source: Extracted from beaver castor sacs
   iv. Common additive: Often preserved with sodium benzoate
f) Amyl acetate (banana flavor)
   i. Risk: No known risks
   ii. Benefit: No known benefits
   iii. Source: Can be distilled from bananas
   iv. Common additive: Often accompanied by ethyl alcohol as a solvent
g) Benzaldehyde (almond flavor)
   i. Risk: No known risks
   ii. Benefit: No known benefits
   iii. Source: Extracted from almonds and cinnamon oil
   iv. Common additive: Often stabilized with sodium metabisulfite
h) Linden ether (honey flavor)
   i. Risk: No known risks
   ii. Benefit: No known benefits
   iii. Source: Derived from linden flowers
   iv. Common additive: Often accompanied by propylene glycol as a solvent
i) Massoia lactone (coconut flavor)
   i. Risk: No known risks
   ii. Benefit: No known benefits
   iii. Source: Extracted from the bark of the Massoia tree
   iv. Common additive: Often accompanied by ethyl alcohol as a solvent
j) Acetoin (butter flavor)
   i. Risk: No known risks
   ii. Benefit: No known benefits
   iii. Source: Naturally occurring in various foods, including butter and yogurt
   iv. Common additive: Often accompanied by propylene glycol as a solvent
k) Spice extracts
   i. Risk: No known risks
   ii. Benefit: May contain antioxidants and other beneficial compounds
   iii. Source: Various spices such as cinnamon, nutmeg, and clove
   iv. Common additive: Often preserved with potassium sorbate
l) Fruit extracts
   i. Risk: No known risks
   ii. Benefit: Rich in antioxidants, may have antimicrobial properties
   iii. Source: Various fruits such as strawberries, apples, and oranges
   iv. Common additive: Often preserved with citric acid
m) Vegetable extracts
   i. Risk: No known risks
   ii. Benefit: Contain various micro and phyto nutrients, and beneficial compounds
   iii. Source: Various vegetables such as carrots, celery, and onions (earthy, roots, etc.)
   iv. Common additive: Often preserved with ascorbic acid
n) Herb extracts
   i. Risk: No known risks
   ii. Benefit: May have various health-promoting properties depending on the herb
   iii. Source: Various herbs such as basil, oregano, and thyme
   iv. Common additive: Often preserved with rosemary extract
o) Bark extracts
   i. Risk: No known risks
   ii. Benefit: May contain beneficial compounds, but specific effects vary
   iii. Source: Various tree barks such as cinnamon and pine
   iv. Common additive: Often accompanied by glycerin as a solvent
p) Bud extracts
   i. Risk: No known risks
   ii. Benefit: No known benefits
   iii. Source: Various plant buds such as clove buds
   iv. Common additive: Often preserved with potassium sorbate
q) Root extracts
   i. Risk: No known risks
   ii. Benefit: No known benefits
   iii. Source: Various plant roots such as ginger and licorice
   iv. Common additive: Often accompanied by ethyl alcohol as a solvent
r) Leaf extracts
   i. Risk: No known risks
   ii. Benefit: No known benefits
   iii. Source: Various plant leaves such as mint and tea leaves
   iv. Common additive: Often preserved with citric acid
s) Dairy product extracts
   i. Risk: Potential allergen for those with dairy allergies
   ii. Benefit: No known benefits
   iii. Source: Various dairy products such as milk and cheese
   iv. Common additive: Often preserved with sodium benzoate
t) Meat extracts
   i. Risk: Potential allergen for those with specific meat allergies
   ii. Benefit: No known benefits
   iii. Source: Various meats such as beef and chicken
   iv. Common additive: Often preserved with sodium nitrite
u) Poultry extracts
   i. Risk: Potential allergen for those with poultry allergies
   ii. Benefit: No known benefits
   iii. Source: Various poultry sources such as chicken and turkey
   iv. Common additive: Often preserved with sodium erythorbate
v) Seafood extracts
   i. Risk: Potential allergen for those with seafood allergies
   ii. Benefit: No known benefits
   iii. Source: Various seafood sources such as fish and shellfish
   iv. Common additive: Often preserved with sodium benzoate
w) Egg extracts
   i. Risk: Potential allergen for those with egg allergies
   ii. Benefit: No known benefits
   iii. Source: Eggs from various poultry sources
   iv. Common additive: Often preserved with potassium sorbate
x) Coumarin (found in cinnamon)
   i. Risk: Toxic in high doses
   ii. Benefit: No known benefits
   iii. Source: Found naturally in cinnamon, tonka beans, and some other plants
   iv. Common additive: Often accompanied by propylene glycol as a solvent
y) Safrole (found in sassafras)
   i. Risk: Toxic in high doses
   ii. Benefit: No known benefits
   iii. Source: Found naturally in sassafras root bark and some other plants
   iv. Common additive: Often accompanied by ethyl alcohol as a solvent
z) Turmeric (curcumin)
   i. Risk: May cause upset stomach, nausea, or diarrhea; potential for liver injury
   ii. Benefit: Anti-inflammatory and antioxidant properties; may help with arthritis, mood disorders, and metabolic syndrome
   iii. Source: Root of the Curcuma longa plant
   iv. Common additive: Often combined with black pepper extract (Piperine) to increase bioavailability
aa) Vanilla
   i. Risk: No known risks in normal amounts
   ii. Benefit: Contains antioxidants
   iii. Source: Vanilla orchid pods
   iv. Common additive: Often preserved with alcohol
bb) Peppermint
   i. Risk: May cause heartburn in some individuals
   ii. Benefit: May help with digestive issues and headaches
   iii. Source: Peppermint plant leaves
   iv. Common additive: Often accompanied by menthol
cc) Ginger
   i. Risk: May interact with blood-thinning medications
   ii. Benefit: May help with nausea and inflammation
   iii. Source: Ginger root
   iv. Common additive: Often preserved with citric acid
dd) Garlic
   i. Risk: May increase bleeding risk in high doses
   ii. Benefit: May have cardiovascular benefits
   iii. Source: Garlic bulbs
   iv. Common additive: Often preserved with citric acid

In summary and when considering general concentration and usage of natural flavors, RNS AI recognizes that natural flavors are often highly concentrated, which allows for reduced usage rates in food products. This concentration means that even smaller amounts are needed to achieve the desired flavor profile, further minimizing any potential nutritional impact.

However, while RNS AI observes that natural flavors themselves may not directly contribute to nutritional value, they can indirectly affect the nutritional profile of so-called naturally flavored food products in several ways:

Enhancing Healthier Options: RNS AI sees that natural flavors can in fact play a crucial role in making healthier food options more palatable. For example:
a. Taste: May help balance taste profiles in reduced-fat, sugar, or salt products.
b. Appeal: In diet sodas, natural flavors can mask off-tastes from artificial sweeteners, making lower-calorie options more appealing to tastes and palates.
c. Offsets: For protein-enriched foods, natural flavors can help reduce off-notes associated with added nutrients, making these healthier options more enjoyable.

Improving Nutritional Formulations: RNS AI reviews nutritionally enriched foods like meal replacement beverages or protein bars, natural flavors can help mitigate unpleasant tastes from added vitamins, minerals, or proteins. Manufacturers may create seemingly more nutritious products without sacrificing taste.
a. Considerations: It's important to note that while natural flavors may not directly impact nutrition, they are not necessarily healthier than artificial flavors. Both types of flavors undergo processing and may contain additional chemicals or preservatives, and despite claims of being “healthy” or “clean”.

RNS AI aligns with users, and as an example for consumers concerned about additives, choosing whole, unprocessed foods and flavoring them naturally (e.g., adding fresh fruit to plain yogurt) remains the most straightforward way to control ingredient intake, maintaining nutritional value.

In conclusion, while most natural flavors themselves do not significantly affect the nutritional value of food products, RNS AI see that they can play a role in making healthier or nutritionally enhanced foods more palatable, potentially encouraging better dietary choices.

D. Common Natural Flavors: Five common natural flavors, sources, associated health impacts:

Natural flavors are derived from various plant and animal sources and can offer potential health benefits. Here's an overview example of five common natural flavors, sources & health impacts:
a) Annatto—Source: Seeds of the achiote tree (Bixa Orellana); Health Benefits:
   i. Rich in antioxidants like carotenoids, tocotrienols, and vitamin A
   ii. Potential antimicrobial properties
   iii. May aid digestion due to fiber content
   iv. Possible bone-strengthening effects from calcium
   v. May support eye health
   vi. Potential skin healing properties
b) Beet Juice—Source: Beetroot (Beta vulgaris); Health Benefits:
   i. May lower blood pressure
   ii. Potential to boost stamina during exercise
   iii. Rich in nitrates, which can improve blood flow
   iv. Good source of folate, potassium, vitamin C, and fiber
   v. Contains antioxidants
c) Fruit Extracts—Source: Key Fruits (e.g., berries, citrus, pomegranates; Health Benefits:
   i. Rich in antioxidants
   ii. Potential antimicrobial properties
   iii. May help prevent chronic diseases like cardiovascular disease and Alzheimer's
   iv. Possible anti-inflammatory effects
   v. Some extracts may have anti-cancer properties
d) Caramel Coloring—Source: Heat-treated sugars; Health Considerations:
   i. Some types may contain potentially harmful compounds (2-MEI and 4-MEI)
   ii. Possible effects on blood pressure
   iii. Potential immune system impacts
   iv. May cause allergic reactions in some individuals
e) Turmeric—Source: Curcuma longa plant root. NOTE: Vegetable extracts generally offer similar benefits to fruit extracts; including antioxidants & nutrients; Health Benefits:
   i. Contains curcumin, a powerful anti-inflammatory and antioxidant compound
   ii. May help ease arthritis symptoms
   iii. Potential to protect against heart disease
   iv. May boost mood and memory
   v. Possible anti-aging effects
   vi. May help combat oxidative stress
   vii. Potential benefits for joint pain, metabolic syndrome, and anxiety

Overall, RNS AI creates an integrated user-aligned platform, which aims to create and encourage actionable transparency (hence “Eating without the Reading”), while educating or exposing users to the many purposeful benefits, and known or unintended risks associated with foods and products consumed, each and all relevant to health objectives, risks or actionable uses or concerns (implicit or explicit). Many natural additives offer functional properties beyond their primary purpose, such as antioxidant or antimicrobial effects. For instance, some natural colorants also act as preservatives and may confer health benefits when consumed regularly.

However, natural does not always mean safe or beneficial. Some natural additives can cause allergic reactions or other adverse effects in sensitive individuals. e.g., sulfites used in wine or packaged vegetables may trigger respiratory issues in some people. Additionally, the stability of natural additives can be a concern or may lead to undesirable changes in food quality.

While natural ingredients generally align with consumer preferences for clean labels (RNS AI assesses claims and compliance), it's important to note that regulations and safety assessments for natural additives vary globally, with some substances permitted in certain countries but banned in others. Ultimately, the health impacts of natural additives depend upon the specific ingredient, its source, processing methods, and user-aligned or defined sensitivities, all of which can be evaluated and scored by RNS, its systems, software, access points and IRX AI platforms.

In conclusion, RNS AI assesses all natural additives, preservatives, sweeteners, colors & flavors, acknowledging that they are increasingly popular alternatives to artificial ingredients in foods or beverages. And, while perceived as healthier options, natural ingredients can have both benefits and risks.

(15) Packaging: RNS AI evaluates the impacts of food packaging on objective or user-aligned health, diet, risks or benefits, requiring a seamless and integrated approach in creating a holistic score. RNS AI considers multiple sourcing and supply chain factors, today and into the future.

RNS AI recognizes that not only does packaging play a crucial role in preserving food quality, safety, and extending shelf life, it also presents potential health risks and environmental concerns.

As an example, chemical leaching from packaging materials, particularly plastics, can introduce harmful substances like BPA, phthalates, and PFAS into foods, potentially affecting human health over time. Conversely, food packaging also offers benefits such as portion control, nutritional labeling, and protection from contamination, which can positively influence dietary choices and food safety (user-aligned risks).

RNS AI assesses the cost-effectiveness and availability of different packaging types, weighting packaging types by products or foods consumed against their environmental impacts and potential health risks. For instance, while plastic packaging is often economical and widely available, its environmental impacts and potential for chemical migration raise concerns.

Conversely, more sustainable options like biopolymers may offer health and environmental benefits, but are often costlier and less readily available. Ultimately, the evaluation of packaging impacts requires balancing these various factors to find solutions that protect both human health and the environment, while remaining economically viable and accessible to consumers. RNS AI can assist diverse users such as consumers, companies and agencies with scoring and evaluating all impacts (benefits and risks), weighted against user-aligned objectives, combined with economic and environmental impacts, regionally and globally.

Critically, food and consumable product packaging (by brands, products, purpose) plays a crucial role in preserving food quality, ensuring safety, and facilitating distribution. However, the materials used for packaging can vary significantly in terms of health risks, environmental impacts, and suitability for different food types.

Below and ongoing is a comprehensive overview of various types of food packaging that RNS AI considers, as well as their uses, associated health risks, and environmental considerations.

Types of Food Packaging: RNS AI weights and considers all types, sources and uses of packaging and direct and indirect impacts to foods or other consumables.
A. Aseptic Packaging: RNS AI Observations
   a) Uses: Commonly used for dairy products (milk, yogurt), juices, soups, and sauces.
   b) Health Risks: Generally considered safe as it does not require preservatives; however, concerns exist regarding chemical leaching from the layers used in manufacturing.
   c) Environmental Impact(s): More eco-friendly than some alternatives due to reduced need for refrigeration and longer shelf life without preservatives.
B. Plastic Packaging: RNS AI Observations
   a) Uses: Snacks (chips), beverages (soft drinks), ready-to-eat (RTE) meals & frozen foods.
   b) Health Risks: High risk of chemical leaching (e.g., BPA, phthalates) especially when heated or in contact with fatty or acidic foods. PFAS (forever chemicals) are also a concern due to their persistence in the environment and potential health effects.
   c) Environmental Impact(s): Significant contributor to pollution; plastic takes hundreds of years to decompose and is often not recyclable due to contamination.
C. Glass Packaging: RNS AI Observations
   a) Uses: Ideal for sauces, jams, beverages (beer, wine), and dairy products.
   b) Health Risks: Generally safe and inert; however, production has a high carbon footprint.
   c) Environmental Impact(s): Recyclable but energy-intensive to produce. Glass bottles are more environmentally impactful than some plastics, if considering full life-cycle impacts.
D. Metal Cans: RNS AI Observations
   a) Uses: Commonly used for vegetables, fruits, soups, and meats.
   b) Health Risks: Potential lead contamination, tin coatings; BPA can linings also a concern.
   c) Environmental Impact(s): Recyclable but energy-intensive to produce; recycling aluminum is less impactful than producing new cans.
E. Paperboard and Cardboard: RNS AI Observations
   a) Uses: Used for cereals, frozen foods, and takeout containers.
   b) Health Risks: Generally low risk unless treated with harmful chemicals; concerns about inks and coatings that may leach into foods.
   c) Environmental Impact(s): Biodegradable and recyclable but can contribute to deforestation if not sourced sustainably.
F. Flexible Packaging: RNS AI Observations
   a) Uses: Includes pouches for snacks, ready meals, and bulk items.
   b) Health Risks: Lower risk compared to rigid plastics, but still potential for leaching depending on material composition.
   c) Environmental Impact(s): Often lighter and uses less material than rigid packaging; however, recycling rates are low due to contamination.
G. Clamshell Packaging: RNS AI Observations
   a) Uses: Commonly used for fresh produce like berries and salads.
   b) Health Risks: Made from plastics that may leach chemicals; less risk compared to other plastic forms due to shorter contact times with food.
   c) Environmental Impact(s): Difficult to recycle; contributes to plastic waste.
H. Trays: RNS AI Observations
   a) Uses: Used for meats and ready-to-eat meals.
   b) Health Risks: Similar risks as flexible packaging; potential chemical leaching from plastic materials.
   c) Environmental Impact(s): Often not recyclable due to food residues' contamination.
I. Wrappers: RNS AI Observations
   a) Uses: Commonly used for candies and snack bars.
   b) Health Risks: Risk of chemical migration from wrappers into food items.
   c) Environmental Impact(s): Typically single-use and contribute significantly to waste. As of today, RNS AI assesses the above nine packaging types represent a majority worldwide.

Risks by Types of Food Packaging: RNS AI Health Risk Ranking by Packaging Type(s) and Related Risk Concerns: NOTE—Storage Climates and Refrigeration also impact Packaging effectiveness and actual Shelf Life of any Food Products; RNS AI assesses Brands, Products and Packaging Types used; short and long-term impacts; stored or prepared as is.
a) Plastic Packaging (high risk of chemical leaching)
b) Metal Cans (risk of lead/BPA)
c) Aseptic Packaging (moderate risk)
d) Flexible Packaging (lower risk)
e) Glass Packaging (generally safe)
f) Paperboard/Cardboard (low risk)

Impacts by Types of Food Packaging: RNS AI weights/scores Environmental Impact Rankings
a) Plastic Packaging (most environmentally harmful)
b) Metal Cans (high energy use)
c) Glass Packaging (high carbon footprint)
d) Flexible Packaging (moderate impact)
e) Paperboard/Cardboard (least impactful if sourced sustainably)

This comprehensive overview highlights diversities of food types, packaging options and uses available globally today along with respective health risks and environmental impacts, each and all considered by RNS AI (by Packaging Types).

As RNS AI users become more aware of these factors, there is a growing global demand for safer and more sustainable clean packaging solutions within the food supply chain.

Based upon persistent RNS AI search results and analyses, here's a comprehensive summary list of food packaging types, including relative use market shares, studied health risks, observed environmental impacts, estimated percentages of global food supply chain utilization, and an estimated number of items by packaging type (supports user-aligned scoring & impacts): Market Utilization by Types of Food Packaging: (type, source, brand, product, % use)
a) Plastic Packaging (Share % and Use % differ based upon Purpose)
   i. Market share: 41.6% of global food packaging industry. (types may be combined)
   ii. Health risks: High risk of chemical leaching (e.g., BPA, phthalates, PFAS)
   iii. Environmental impact: Significant contributor to pollution; slow to decompose
   iv. Estimated % of global food supply chain: ~40%
   v. Estimated items: Over 1 trillion units globally, annually
b) Paper & Paper-based Packaging: (Share % and Use % differ based upon Purpose)
   i. Market share: 30% of global food packaging market. (types may be combined)
   ii. Health risks: Generally low risk unless treated with harmful chemicals
   iii. Environmental impact: Biodegradable & recyclable; but contributes to deforestation
   iv. Estimated % of global food supply chain: ~25%
   v. Estimated items: 500-700 billion units annually
c) Glass Packaging/Jars (Share % and Use % differ based upon Purpose)
   i. Market share: 10% of global food packaging market. (types may be combined)
   ii. Health risks: Generally safe—glass is inert (source by source, case by case given lids)
   iii. Environmental impact: Recyclable, but energy-intensive to produce (by country)
   iv. % of global food supply chain: ~10%
   v. Estimated items: 830 billion units in 2024, projected to reach 1.03 trillion by 2029
d) Metal Packaging (Share % and Use % differ based upon Purpose)
   i. Market share: 15% of food packaging market. (types may be combined)
   ii. Health risks: Potential lead contamination from tin coatings; BPA in can linings
   iii. Environmental impact: Recyclable but energy-intensive to produce
   iv. % of global food supply chain: ~15%
   v. Estimated items: 364.4 billion cans in 2014, estimated at 430 billion in 2024
e) Flexible Packaging (Share % and Use % differ based upon Purpose)
   i. Market share: 44.3% of food packaging market by type. (types may be combined)
   ii. Health risks: Lower risk compared to rigid plastics but still potential for leaching
   iii. Environmental impact: Often lighter and uses less material than rigid packaging
   iv. % of global food supply chain: ~8%
   v. Estimated items: 200-300 billion units annually
f) Rigid Packaging (Share % and Use % differ based upon Purpose)
   i. Market share: 33.4% of food packaging market by type. (types may be combined)
   ii. Health risks: Varies depending on material used.
   iii. Environmental impact: Generally more resource-intensive than flexible packaging
   iv. % of global food supply chain: ~2%
   v. Estimated items: 50-100 billion units annually

This list covers approximately 100% of the global food supply chain packaging types. The estimated number of items for each packaging type is based on available data and industry trends. Actual figures may vary due to the dynamic nature of the global food packaging market.

Today, food packaging and its negative impacts (short and long-term impacts to people & planet) on health are under evermore scrutiny globally. Critically, RNS AI supports user-aligned decision-taking and actionable insights, aligned with specific or acceptable health impacts to foods, including any adverse or beneficial contributions of packaging. Importantly, RNS AI research exposes users to actionable risks/impacts of food packaging chemicals to human health.

As an example, a comprehensive study published in September 2024 in the Journal of Exposure Science and Environmental Epidemiology has shed light on the extent of human exposure to food contact chemicals (FCCs), which are evaluated and available through IRX FOODSCORE outputs for specified products, production, preservation, packaging and preparation risks thru Relevant Risk Scores (RRS) in support of user-aligned vantage points.
A. Packaging Key Findings: IRX FOODSCORE provides RRS insights aligned with users, assessing short and long-term risks, as well as storage and preparational uses.
   a) Widespread Exposure: Researchers identified 3,601 chemicals from food packaging in human samples, including blood, hair, and breast milk. RNS considers published research lists of all chemicals considered to impact human health, as part of any RNS food score.
   b) Health Risks: Many of these chemicals are known as endocrine disruptors and carcinogens, including PFAS, phthalates, and bisphenols. RNS AI extracts reports and histories by packaging sources, types, brands and products in reaching risk assessments.
   c) Leaching Factors: High temperatures and fatty, acidic foods increase chemical leaching from plastic and paper packaging into food. RNS breaks out and evaluates likely leaching sources and potential impacts for user-selected foods “as is” and “as prepared” in any score.
B. Packaging-specific Concerns: IRX FOODSCORE assesses RRS, product-specific and packaging-related health risks or impacts to food and products delivered for specific users.
   a) PFAS (Per- and Polyfluoroalkyl Substances)
      i. Known as forever chemicals due to persistence in the environment and human body.
      ii. Linked to cancers, liver damage, and developmental defects in children.
   b) Bisphenols (e.g., BPA)
      i. Linked to hormone issues, high blood pressure, type 2 diabetes, cardiovascular disease
   c) Phthalates: (plastics used for food packaging, and other non-food-related uses)
      i. Linked to hormone and reproductive issues.
   d) Research Gaps: RNS AI continuously monitors all chemicals and packaging studies.
      i. Many chemicals' potential hazards have not been sufficiently investigated.
      ii. Actual number of FCCs present in humans is likely higher than currently detected.
C. Regulatory Implications: IRX FOODSCORE assesses product-specific packaging-related health risks & impacts to food/products through RRS for unique users. Recent studies expose current regulations in the US (globally) as insufficient to protect consumers from chemical exposures in food packaging (raw, stored, prepared). Consequently, legislators are advocating for improved safety measures and alternatives thru regulatory bodies, protecting free markets and consumers. Critically, RNS & RRS provide user-aligned transparency to ensure informed decision-making.

(16) Weighting User-Aligned Inputs: RNS AI assimilates a considerable amount of data, and to best use this data, RNS AI weights & calibrates it to ensure it is getting the most accurate picture possible. RNS AI approaches data weighting like adjusting the volume on different instruments in a band. Sometimes you need to turn up the bass and lower the treble to get the perfect sound.
A. Weighting data is important because:
   a) It reduces sampling bias
   b) It makes our results more applicable to the real world by enhancing generalizability
   c) It improves the quality of our data by giving more importance to reliable sources, achieving better insights
   d) It helps meet specific research objectives
B. Weighting data inputs; RNS AI utilizes several methods, including but not limited to:
   a) Probability Weights: Assigned based on the likelihood of inclusion in the survey or dataset, and by known historical studies and observations relevant to specific impacts.
   b) Non-Response Weights: Address issues of respondents who don't participate, reducing non-response bias. This also applies to single questions with no response, as weights (by question/category) will be dynamically redistributed without bias.
   c) Post-Stratification Weights: Adjust the sample to match known population characteristics.
   d) RIM Weighting: Used when the sample doesn't adequately cover all possible combinations of multiple variables.
   e) Calibration Weighting: Adjusts weights based on known population totals for specific variables.
   f) Propensity Score Weighting: Reduces selection bias in observational studies
   g) Cell-based Weighting: Applies specific weights to predefined demographic combinations, ensuring the sample matches known population distributions.
   h) Raking: Iteratively adjusts weights for multiple variables when their joint distribution is unknown, balancing the sample across various demographic factors.
   i) Matching: Pairs cases from the study dataset with representative cases from a reference dataset, creating a balanced sample that mirrors the target population.
   j) Logistic Regression Modelling: Uses statistical modeling to calculate weights that correct for selection bias, particularly useful in complex sampling scenarios.
   k) Inverse Variance Weighting: Assigns weights inversely proportional to the variance of each subpopulation, giving more weight to more precise estimates.
   l) Frequency Weighting: Replicates each observation a specified number of times, useful when dealing with aggregated data or count data.
   m) Survey Weights: Indicates how many individuals in the population each sampled unit represents, crucial for inferring population characteristics from survey data.
   n) Analytical Weights: Specifies the precision of each observation, often used in regression analyses where observations come from groups with different variances.
   o) Full Data Update (FDU): A machine learning (ML) technique that improves model updating efficiency by selectively processing the most informative data points.

Weighting user-aligned inputs in RNS AI-enabled and machine learning (ML) environments is about making our nutritional technology platform work better for real, and diverse users. It creates systems that understand the nuances of country and culturally-aligned human health, preferences, and behaviors. By doing this right, we can offer insights and advice that are truly personalized or regionalized geo-demographically for target products and populations, improving health outcomes and quality of life for people around the world.

(17) Global Product Assessments: The IRX FOODSCORE system, methods and software and its proprietary disparate data refinery and algorithms enable a comprehensive general and generative AI environment in support of a dynamic and persistent assessment of the specific issue or holistic health, nutritional and risk impacts related to any global food, beverage or consumable products.

A. IRX-AI enabled Proprietary Approach: considers multiple factors across the entire global food supply chain and integrates the use of AI, machine learning (ML) and data lakes to capture and analyze dynamic and ever-growing disparate data sources (private and public) combined with analyses and relevant research findings (scientific, chemical, genetic, biological, medical, nutritional, others) to provide robust and relevant consumer or user-aligned evaluations of all food, beverage and consumable products. Quantum computing (QC) will also be employed as practical and available.

As defined prior and below is a summary outline of such a proprietary IRX FOODSCORE, IRX AI and RNS AI framework in support of global product assessments:
a) IRX-AI Data Collection and Analyses:
   i. Agricultural Practices (production sources: company, brand and product-aligned)
      a. Soil histories, treatment and conditioning (chemical & contamination history)
      b. Pesticides and fertilizers usage data (known, observed or prevailing practices)
      c. Organic vs. conventional farming methods (country and culture aligned)
      d. Sustainable agriculture practices (as reported, agency verified or certified)
   ii. Harvesting and Storage (RNS weighted production yields or inspection records)
      a. Harvesting techniques (country and culture aligned)
      b. Storage conditions and duration (prevailing conditions, climate and types)
      c. Post-harvest treatments (pesticides, preservatives, processing, environmental)
   iii. Processing and Packaging (Packaging data defined in number 15 above)
      a. Processing methods (e.g., cold or heat treatments, stripping, fermentation, etc.)
      b. Preservation techniques (natural, organic, artificial or chemical)
      c. Packaging materials and methods (see packaging, number 15 above)
   iv. Ingredient Analysis (RNS assesses all sourcing, processing, preservation practices)
      a. Organic vs. artificial ingredients (as disclosed, verified, analyzed or certified)
      b. Nutrient content and bioavailability (source data, analyses, studies)
      c. Presence of additives and preservatives (as disclosed, verified or certified)
b) IRX-AI Research Integration:
   i. Global Scientific Literature Review and Sourcing (country relevant, validated)
      a. PubMed and other credible study sources
      b. Peer-reviewed studies on health impacts of ingredients and processes
      c. Meta-analyses of nutritional research
      d. Toxicological assessments of food components
   ii. Regulatory Guidelines (Contemporary or prevailing agency guidelines)
      a. National and international food safety standards (global or by country)
      b. Recommended daily allowances for nutrients (target population-relevant)
      c. Acceptable daily intakes for additives or contaminants (risk-reward modeling)
c) IRX-AI Health and Nutrition Impact Assessment(s):
   i. RNS AI Nutrient Profiling (as defined throughout this glossary)
      a. Evaluation of macro and micronutrient content (declared, or certified)
      b. Assessment of energy density (relevant, absorbed, and efficacy)
      c. Analysis of fiber and phytonutrient content (considering 200+ nutrients)
      d. Relevant Nutrition Scoring (RNS) functional, purpose, bio-availabilities
   ii. RNS AI and RRS Contaminant Evaluation (Pesticides, Packaging, Preservation)
      a. Pesticide, mineral or chemical residue or leaching levels (reported or tested)
      b. Heavy metal contamination (environmental, mining, or industrial exposures)
      c. Presence of antibiotic residues or growth hormones (as reported or analyzed)
   iii. RNS AI Processing Impact Evaluation (types and treatments; verified or certified)
      a. Specific processing practices effects; nutrient retention, dilution, contamination
      b. Formation of potentially harmful compounds during processing (by method)
      c. Impacts of preservation methods on nutritional quality, efficacy, functionality
d) IRX FOODSCORE and RNS-AI Proprietary Scoring Methodology:
   i. Leverage of IRX FOODSCORE, IRX AI and RNS-AI Scoring Algorithms
      a. Integration of disparate traditional and non-traditional nutritional factors
      b. Weighting and distribution of positive and negative (risk) product attributes
      c. Consideration of serving sizes, stated uses, purposes and functional impacts
   ii. RNS Scoring Components and Distinct Contributions (not all inclusive)
      a. Nutrient density score (macro/micro/phyto by purpose, interactions, functions)
      b. Contaminant risk score (any source: ingredients, pesticides, processing (additives or chemicals), packaging (leaching), preservation (artificial), other.
      c. Processing impact score: weighting processing-specific impacts/contributions
      d. Prevailing standards compliance score: Assessing 3^{rd} Party voluntary compliance or governmental agency standards of use or disclosure compliance. e.g., ISO Standards (International Organization of Standardization) or GRAS (Generally Recognized as Safe); both dynamically change over time, transparency Sustainability score: evaluating environmental impacts and product benefits e. Benchmark score: comparative categories or product impact assessments
e) IRX-AI Implementation of RNS Scoring System:
   i. Specific Product Assessment (specific user specified; defined use, health purpose)
      a. Application of RNS-AI scoring algorithm; individual user-aligned products
      b. Categorization of products based on nutritional quality, function and purpose
      c. Comparative analysis and benchmarking within food categories (compliance)
   ii. User (e.g., Consumer) Communication of RNS Score Actionability and Function
      a. Development of clear and intuitive labeling or CPG manufacturer compliance certification standards by specific or general health standards. Enables application delivery of IRX FOODSCORE “Eating without the Reading” product assessment transparency of both beneficial and detrimental attributes, which are user-aligned.
      b. Integration of scores and insights into product packaging and marketing, which in effect replace unreliable traditional nutrition panels, ingredient listings and unenforced, non-transparent product health claims.
      c. Educational initiatives to help diverse users consumers interpret scores and user-aligned vantage points, insights and relevant attributes for specified health goals (specific or general).
   iii. Industry Impact: IRX FOODSCORE—RNS disrupting global food supply chains.
      a. Incentivizing product reformulation and optimization for better scores/health.
      b. Promoting transparency in food supply chains to ensure removal of end-user risks; transparency of understandings of risks and benefits (beyond taste or price)
      c. Encouraging adoption and deployment of sustainable and healthy practices
      d. Offering compliance and risk certification standards and benchmarking
      e. Providing governing agencies, legislators, retailers and consumers alike with a transparent and aligned product assessment currency (Scores) focused on general health optimization and specific health function and purpose; Tangible metrics.
f) IRX-AI Continuous Improvement and Adaptation:
   i. Monitoring and Evaluation (tracking, trending and compliance certifications)
      a. Tracking changes in product scores over time (general or specific health)
      b. Assessing impacts on consumer behaviors and objective health outcomes
      c. Identifying trends and improvements in global food industry practices
   ii. User-aligned Stakeholder Engagement (IRX FOODSCORE has 12 User Types)
      a. Collaboration between governing agencies, food manufacturers and retailers
      b. Supports consultative currency for nutrition experts & public health officials
      c. Supports incorporation of consumer-specific feedback, purpose & preferences
   iii. Regulatory Alignment: IRX FOODSCORE support regulatory reformation
      a. RNS AI provides common language, “currency of exchange” for compliance between agencies and producers with ever-evolving food labeling regulations.
      b. RNS AI-available insights support evidence-based food industry policy changes and legislative health objectives for general and at-risk populations.
      c. RNS AI supports global harmonization of nutritional, health & risk standards.

This simplified summary of our IRX FOODSCORE and RNS AI framework provides a global, comprehensive approach to assessing the health and nutritional impacts of food products.

By integrating disparate data from across the food supply chain, incorporating the latest scientific and medical research, and by utilizing a proprietary IRX-AI scoring methodology, this product assessment approach offers a robust tool for evaluating the overall quality of any food products.

This proprietary IRX FOODSCORE health and nutrition scoring methodology plays a crucial role throughout the food supply chain for any consumable product, as it is disruptive by design. Yet it uniquely enables, empowers and aligns disparate users around fully integrated purpose-driven health objectives, previously unavailable to any user, by:
1. Synthesizing complex and seemingly unrelated data into easily understandable scores
2. Providing standardized, compliance methods for comparing diverse global food products
3. Encouraging transparency and improvement throughout the global food industry
4. Empowering consumers to make informed, transparent and purposeful dietary choices

By implementing the IRX FOODSCORE, governing bodies, food manufacturers, retailers, medical professionals and consumers can work collaboratively to promote healthier, more purposeful, and more sustainable products and food systems, ultimately contributing to improved public health outcomes for all involved.

(18) Dietary Choices: For RNS AI, it's crucial to understand how various dietary choices affect nutrition and health goals. Here are some key dietary choices and their implications that RNS AI considers: (Not all inclusive)

A. Whole Foods vs. Processed Foods:
a) Whole foods: foods that are minimally processed and do not contain added sugars, preservatives, or artificial ingredients. Examples include fruits, vegetables, whole grains, nuts, and seeds. These foods are generally rich in nutrients, fiber, and beneficial plant compounds, however relevancy to health goals or concerns still needs to be determined.
b) Processed foods: foods that have been altered from their natural state for safety or convenience. Highly processed foods often contain added sugars, sodium, and unhealthy fats, or have been stripped of essential vitamins or benefits, and potentially negatively impacted by the processes themselves. Processed examples include packaged snacks, soft drinks, and ready-to-eat meals.

Choosing whole foods over processed foods can significantly improve overall nutrition while supporting various health goals, such as weight management, heart health, and diabetes prevention. However, user-aligned objectives & purpose need to be considered; RNS AI creates actionable transparency to dietary drivers, real food impacts on health and user-aligned priorities.

B. Plant-Based vs. Animal-Based Diets:
a) Plant-based diets: focus on foods derived from plants, including fruits, vegetables, nuts, seeds, oils, whole grains, legumes, and beans. These diets can be rich in fiber, vitamins, minerals, and antioxidants. We often hear dietary terms like vegetarian/vegan, which can be further evaluated for strict compliance and benefit or risk through RNS AI insights.
b) Animal-based diets: include a higher proportion of foods derived from animals, such as meat, fish, eggs, and dairy products. These foods can be good sources of protein, vitamin B12, iron, and zinc. However, we have all heard or used the terms “eat a balanced diet” or “everything in moderation”, which can be further verified, measured or purposefully applied and assessed through the use of RNS AI scores.

Shifting towards a more plant-based diet has been associated with lower risks of heart disease, certain cancers, and type 2 diabetes. However, balanced animal-based diets can also be nutritious when consumed in moderation and contain more sources of complete proteins. Ultimately, individual consumers need to assess and determine diets that deliver purposeful calories, as relevant and necessary to maintain or to improve health situations and outcomes.

C. Low-Carb vs. High-Carb Diets:
a) Low-carb diets: restrict carbohydrate “carbs” intake, often focusing on proteins and fats. These diets can be effective for weight loss and blood sugar control in some individuals. The issue becomes that the term carbohydrates is too broad and general, and that there are healthy carbs required for proper and healthy bodily functions including immunities.
b)High-carb diets: emphasize carbohydrates as the primary source of calories. When based on whole grains, fruits, and vegetables, high-carb diets can be rich in fiber and nutrients

The optimal carbohydrate intake varies depending on individual health goals, metabolic health, activity levels and situational priorities. Eating the right carbs, quantity and quality comes down to user-aligned health objectives, impairments and activities. RNS AI delivers the transparency necessary to take proactive or reactive dietary actions to achieve desired health outcomes.

D. High-Protein vs. Low-Protein Diets:
a) High-protein diets: emphasize protein intake, often from sources like meat, fish, eggs, and dairy. These diets can support muscle growth, weight management, and satiety. RNS AI assesses quality, quantity, macro, micro and phyto nutrients as well as relevant risks.
b)Low-protein diets: contain less protein and may be recommended for certain health conditions, such as kidney disease. However, adequate protein intake is essential for overall health and bodily functions. Thus, RNS AI considers, weights and values user-aligned priorities, benefits & risks in achieving health objectives or purposeful dieting.
   E. Low-Fat vs. High-Fat Diets:
a) Low-fat diets: restrict fat intake, often to manage weight or heart health. However, it's important to include healthy fats in the diet. This concept falls woefully short, as it does not discuss sources, types, quality and quantity of fats. Fats are in fact healthy and require an understanding and exposure of benefit. A human body needs fats to function, which are further considered by RNS AI scores, insights and risks, all user-aligned.
b)High-fat diets: such as the ketogenic diet, emphasize fat as primary source of calories. These diets can be effective for certain health conditions, but may not be suitable for all users. Again, quality and quantity of fats to be consumed need a context, relevancy measure and metrics in place to ascertain benefits or risks, all achieved by RNS AI.
F. Specific Popular Diets and Their Impacts:
   a) Vegan Diet: Excludes all animal products, including meat, dairy, eggs, and even honey. It's based entirely on plant foods, which is assessed in whole or part by RNS AI scores, while considering user-aligned benefits, risks and deficiencies for any period(s) of time;
      i. Nutritional Impact(s): RNS supports user-specific health information and objectives.
         a. High in fiber, vitamins C and E, folic acid, magnesium, iron, and phytochemicals.
         b. Often lower in saturated fat and cholesterol. But types and sources of fat matter.
         c. May reduce vitamins B12 or D, calcium & omega-3 fatty acids if no proper plan.
         d. May result in positive benefits or detrimental risks, if not purposeful, complete and transparent in support of user-driven purpose. RNS AI delivers this actionable transparency with perspectives and suggestive dietary corrections.
      ii. Health Implication(s): RNS AI creates transparency with aligned purpose, necessary to ensure objectives.
         a. Potential benefits for heart health and diabetes management. Actual diet pros/cons
         b. May help with weight loss and reducing cancer risk; need to know risks/benefits.
         c. Requires careful planning to avoid nutrient deficiencies; may exacerbate issues.
   b) Ketogenic (Keto) Diet: The keto diet is a high-fat, very low-carbohydrate diet that aims to put the body into a state of ketosis.
      i. Nutritional Impact: RNS AI evaluates and exposes purpose-aligned benefits or risks.
         a. Very high in fats (70-80% of calories); quantity, quality & sources of fats matter.
         b. Moderate in protein; type/sources, quality & uses define actual benefit or efficacy.
         c. Very low in carbohydrates (typically less than 50 g per day); again quality matters.
      ii. Health Implications: RNS AI exposes both risks and rewards aligned with user-aligned objectives.
         a. Can be effective for short-term weight loss; actual benefit & sustainability matter.
         b. May improve insulin sensitivity; RNS AI supports user-aligned health outcomes.
         c. Potentially beneficial for epilepsy management; user-aligned health objectives.
         d. Long-term effects are still being studied; RNS AI leverages latest actionable study data available at time of analyses or scoring.
   c) Paleo Diet: Aims to mimic the eating patterns of our hunter-gatherer ancestors.
      i. Nutritional Impact: RNS AI captures & considers many benefits of ancient cultures.
         a. High in lean proteins, fruits, vegetables, nuts, and seeds; back to nature benefits.
         b. Excludes grains, legumes, dairy, and processed foods; RNS reaffirms benefits.
         c. Can be high in saturated fats depending on food choices; quality of fats matter.
      ii. Health Implications: RNS AI aligns user goals & objectives considering total health.
         a. May aid in weight loss and improve blood sugar control; foods are low glycemic.
         b. Potential benefits for heart health. RNS AI assesses foods & product compliance.
         c. Risk of calcium deficiency due to dairy exclusion; RNS AI assists user-alignment.
   d) Mediterranean Diet: This diet is based on the traditional eating patterns of countries bordering the Mediterranean Sea. Ancient cultures, climates and crops had resilient immunities, which can be replicated through dietary choices; RNS AI user-alignment.
      i. Nutritional Impact: Nutritional was originally used to mean Natural; RNS AI assesses benefits.
         a. Rich in fruits, vegetables, whole grains, legumes, nuts, olive oil; quality matters.
         b. Moderate in fish & poultry; original definition of balanced diet; RNS AI insights.
         c. Low in red meat and processed foods; Protein is critical for function & immunity.
      ii. Health Implications: Historical cultural benefits to healthy “balanced” eating.
         a. Linked with reduced heart disease risk or stroke. RNS AI creates user-alignment..
         b. May improve cognitive function and reduce risk of Alzheimer's; RNS AI verifies.
         c. Likely benefits for weight management & diabetes prevention; RNS AI verifies.
   e) Gluten-Free Diet: Diet eliminates all gluten; a protein found in wheat, barley, and rye.
      i. Nutritional Impact: RNS AI assesses dietary relevancy and compliance balanced with user-aligned concerns, objectives and health concerns.
         a. Excludes many common grains & processed foods; RNS AI assesses deficiencies.
         b. May be lower in fiber or B vitamins if not properly planned; RNS AI measures.
         c. Can be nutritionally complete if balanced with other whole grains and nutrients.
      ii. Health Implications: RNS AI assesses the user-relevant impacts, benefits or risks.
         a. Essential for people with celiac disease, gluten sensitivity; RNS AI assesses risk.
         b. No proven benefits for those without gluten-related disorders; RNS AI scored.
         c. May lead to nutrient deficiencies if not carefully planned; RNS AI evaluates diets.
   f) Intermittent Fasting: This is not a specific diet but an eating pattern that cycles between periods of eating/fasting; RNS AI assess user-aligned nutritional deficiencies over time.
      i. Nutritional Impact(s): RNS AI accounts for nutritional properties, benefits and risks.
         a. Can lead to reduced calorie intake; RNS AI assesses health risks and shortfalls.
         b. Nutrient intake depends on food choices during eating periods; RNS AI assesses.
      ii. Health Implications: RNS AI assesses all known or studied health implications.
         a. May aid in weight loss & improve insulin sensitivity; RNS AI evaluates benefits.
         b. Potential benefits for cellular repair & longevity; RNS AI ensures user-alignment.
         c. Effects vary greatly depending on specific fasting protocol; RNS AI user-aligned.
   g) Raw Food Diet: This diet consists mainly or entirely of uncooked or unprocessed foods.
      i. Nutritional Impact: RNS AI evaluated user-aligned impacts, benefits, and any risks.
         a. High in fruits, vegetables, nuts, seeds, and sometimes raw animal products
         b. Rich in vitamins, minerals, and enzymes; RNS AI scores dietary sources.
         c. May be low in some nutrients like vitamin B12, iron, calcium; RNS AI identifies.
      ii. Health Implications: RNS AI aligns user reported health information & objectives.
         a. May aid in weight loss & improve digestion; RNS AI aligns nutrition imperatives.
         b. Potential risk of foodborne illness; RNS AI exposes risks of raw animal products.
         c. Often difficult to meet nutritional needs, especially over time; RNS AI assesses.

RNS AI considers these diverse dietary approaches, given that each diet has its own nutritional strengths and challenges. RNS AI can evaluate food choices within the context of these various diets, ensuring that users receive recommendations that align with their chosen dietary approach and objectives, while still meeting their nutritional needs and health goals.

Moreover, RNS AI is flexible enough to accommodate users who may be following modified versions of these diets or combining elements from different approaches. RNS AI flags potential nutrient deficiencies, imbalances or risks that may arise from strict adherence to certain diets, prompting users to seek professional advice when necessary.

By incorporating a comprehensive understanding of various dietary choices, RNS AI provides more personalized, relevant, transparent and effective nutrition scores, insights and suggestions to a diverse global user base from many vantage points.

G. Nutrition Deficiencies and Dietary Risks Deserve Transparency: RNS AI observes diets rich in processed or highly processed foods pose significant health risks, as evidenced by numerous current studies and expert opinions. Processed and highly processed foods are often characterized by long lists of difficult to understand ingredients or chemical additives, and minimal whole food content, many of which are linked to major health concerns.

Scientific research and medical studies indicate that regular consumption of highly processed foods increases risks of obesity, type 2 diabetes, cardiovascular disease, certain cancers (e.g., Colon), and even mental health disorders. Now today, an alarming prevalence of these foods in modern diets—accounting for up to 67% of calories consumed by children and teenagers in the U.S. today alone—has exposed serious concerns about health, both domestically and globally, as this has become a global crisis and epidemic.

RNS AI research shows that western lifestyles are in part to blame, given the past several decade introduction of western foods, convenience foods, take-out & fast foods combined with larger refrigerators & freezers, serving sizes and anytime meal consumption. Altogether, these changes have caused global increases in certain cancers, obesity, diabetes and heart disease.

RNS AI observes that the combination of added sugars, salts, and unhealthy fats (e.g., Oils below), preservatives and artificial ingredients in foods directly lead to chronic inflammation, nutrient deficiencies, and metabolic disturbances, and contribute to higher risks of premature death. Yes, diets are driving death domestically and globally, and a new approach to measuring, monitoring and creating informed and user-relevant transparency is needed.

IRX FOODSCORE and RNS AI deliver that transparency, relevancy and actionable insights to secure change. Oil example below:
a) The Use of “Processed Foods in Processed Foods”: RNS AI Creates Transparency i. RNS AI and related risk analyses recognize that seed oils, which are in fact processed foods from inception, undergo significant manufacturing steps to transform seeds into many everyday cooking oils (See below). In fact, the production of most commercial (and some artisanal) seed oils involves several industrial processes and steps to final products:
   a. Extraction: Seeds are crushed, and oil is extracted using mechanical pressing and/or chemical solvents like hexane.
   b. Refining: The extracted oil undergoes various refining steps, including:
      i. Degumming: Removing phospholipids and other impurities.
      ii. Neutralization: Removing free fatty acids.
      iii. Bleaching: Removing pigments and other compounds.
      iv. Deodorization: Removing volatile compounds that cause odors.
   c. Additives: Manufacturers often add synthetic antioxidants and preservatives, as they help to extend oil product(s) shelf life.
RNS AI recognizes that this extensive processing distinguishes seed oils from minimally processed oils like extra-virgin olive oil, which is simply mechanically pressed without use of chemical solvents or high heat (preserves nutrients without infused risks). While seed oils are derived from natural sources (plant seeds), the final product is far removed from its natural original state due to the aforementioned industrial processing. Sadly, the refining process strips away many of the natural compounds found in seeds, including beneficial antioxidants and vitamins. RNS AI also distinguishes that some seed oils (e.g., those labeled as “cold-pressed” or “extra-virgin,” undergo less processing and may retain more of their natural compounds. However, the majority of commercially available seed oils are highly refined products, which directly impacts nutritional and risk properties. In summary, most seed oils are considered “processed foods” rather than natural or artificial. They typically do not contain added preservatives in pure form, but the refining process itself acts as a form of preservation by removing complex natural compounds that could lead to spoilage. Thus, the use of these oils deserves further attention.
b) Oils use in Processed Foods: Substantive Risks to People Consuming Processed Foods
   i. Seed oils are vegetable oils extracted from various plant seeds through processing methods, as described above. Common examples of seed oils include canola, sunflower, grapeseed, soybean, corn, and safflower oils.
   ii. These seed oils are widely used in food production and cooking due to affordability, availability, and high smoke points.
   iii. Use in Processed Foods (RNS AI evaluates all sources/types of fats used in products)
      a. Seed oils are extensively used in ultra-processed foods, including but not limited to ten common types of convenient ready-to-eat (RTE) prepared foods and snacks:
         i) Snack foods (chips, crackers, tortilla chips, cheese puffs, popcorn, etc.)
         ii) Baked goods (cookies, cakes, muffins, breads, etc.)
         iii) Fried foods (French fries, chicken nuggets, fish sticks, etc.)
         iv) Condiments and dressings (mayonnaise, sauces, salad dressings, etc.)
         v) Frozen meals/Packaged food (entrees, side-dishes, pizza, sandwiches, etc.)
         vi) Canned foods (canned soups, some canned vegetables, meats, fish, etc.)
         vii) Granola bars and protein bars
         viii) Some dairy products and plant-based alternatives
         ix) Energy drinks and soft drinks
         x) Ice Cream and Frozen Desserts (Ice creams, bars, pies, cakes)
      b. Seed oil benefits (RNS AI creates relevant benefit and risk scores; user-aligned):
         i) Rich in omega-6 fatty acids, which can help to improve cholesterol levels or to decrease heart disease risk when used in place of saturated fats.
         ii) Contain vitamin E and phenols; yet these are reduced during processing.
         iii) Affordable and widely available cooking oils with high smoke points.
      c. Seed oil risks (RNS AI creates relevant benefit and risk scores; user-aligned):
         i) Recent PubMed/Other Studies: raise concerns about potential global health impacts from “cheap” fats and processed oils.
         ii) Inflammation: High levels of omega-6 fatty acids contribute to chronic inflammation in a body. RNS AI creates user-aligned risk transparency.
         iii) Colon Cancer: A recent study published in Gut Journal found high concentrations of bioactive lipids in colon cancer tumors, potentially linked to seed oil consumption. RNS AI incorporates all credible research.
         iv) Oxidation: The polyunsaturated fats in seed oils can oxidize, which produces compounds; contributes to RNS AI user-aligned health issues.
         v) Processing: Refining processes may remove beneficial compounds and potentially create harmful chemicals.

In summary, it's important to note that while valid concerns about seed oils exist, user and use-specific moderation and overall dietary patterns play a significant role in objective or responsive health outcomes. Critically, RNS AI actively aligns health objectives or concerns around user-defined purpose(s) proactively or reactively, providing details and support of benefits or risks associated with any foods in support of any dietary practices.

(19) Food Innovations: The seamlessly integrated IRX FOODSCORE system, methods and software deliverables (IRX AI and RNS AI), which are accessible through a SaaS-supported, and generative, interactive IRX AI-enabled advisory environment (with other RNS AI-driven, integrated services & deliverables), altogether facilitate a proprietary and comprehensive food, beverage and consumable product development platform.

This one-of-a-kind product optimization and innovation platform enables country-capable, culturally-aligned, dietary-driven, legislation-compliant and purposeful health-relevant food innovations, proactively or reactively, opportunistically improving health benefits for target global consumers, producers or manufacturers, while mitigating observed, known or potential product consumption risks.

This proprietary persistent developmental platform and functional formulation framework supports general and purposeful health formulations or reformulations through user-aligned and defined objectives, health purposes and dietary drivers, utilizing all available, relevant and pertinent product and purpose-related disparate datasets. Critically and specifically, this IRX AI and RNS AI platform integrates, analyzes and combines all available user and product(s) inputs and objective outcomes with all available datasets, including but not limited to all available formulaic, scientific, biological, medical, nutritional, and historical data related to product-aligned supply chains, ingredients, production practices, food or drug interactions, side-effects, disease-responsiveness, preservation methods, packaging protocols, preparation practices.

The IRX AI and RNS AI platform supports new product innovation practices necessary to establish a “good”, “better”, or “best” model for any ingredients, food products, or consumables enabling evolutionary product innovations, formulations or reformulations aligned with product positioning, purposeful product or brand strategies, and while ensuring mandated or objective health compliance, certifications and outcomes, minimized or maximized as aligned with claims.

### IRX FOODSCORE Framework for Global Food Innovations:

### A. RNS AI Assessment of Current Food Systems:

a) Analytical Framework: The IRX FOODSCORE data refinery enables an analytical framework that evaluates the health, environmental, social, and economic impacts of any food system globally, aligning with product-specific goals, business drivers, consumer priorities, regulatory compliance and health standard(s) objectives, with step-up options for “good”, “better” and “best” product performance metrics (benefits and risks) and target nutritional composition. This includes methodologies such as Life Cycle Assessments (LCA) and Health Impact Assessments (HIA), necessary to understand the implications of reformative recipes or prospective product positioning, purposeful food production, user-aligned and cost-optimized health objectives, relevant nutrition scores, and other specified benefits or resulting product risks.
b) Nutritional Research and Optimization: See footnote (1) under Proprietary System: Relevant Nutrition Scores (RNS's), RNS AI systematically integrates cutting-edge scientific methods and artificial intelligence (AI) to comprehensively assess and optimize the health impacts of foods, consumables and ingredients, and their bio-functional impacts and efficacies. This advanced system leverages multiple AI technologies and data sources to provide nuanced, relevant nutritional and risk-related insights beyond traditional nutrition guidelines and scoring systems. RNS AI evaluates all available relevant ongoing research. (e.g., NIH's Nutrition for Precision Health initiative to gather data on how different populations respond to various dietary interventions; supports product development & innovation tailored to unique health needs).

### B. RNS AI Enablement and Implementation of Innovation Ecosystems:

a) Collaborative Partnerships: RNS AI fosters user-aligned partnerships around nutrition valued-vantage points utilizing RNS AI scores as a nutrition crypto currency of collaboration and exchange among governments, CPG manufactures/businesses, farmers and food supply chains, scientific & nutritional researchers to co-create clean and transparent solutions that are health relevant, and which actionably address specific food challenges and health outcome objectives. The establishment of RNS AI-enabled Food Innovation Hubs can facilitate this collaboration, providing clear direction and transparency initiatives to drive purposeful, constituency-aligned and realistically actionable product innovations or reformations.
b) Functional Regulatory Frameworks: Implement RNS AI-enabled, robust regulatory frameworks that ensure governmental-aligned health objectives and food safety measures, while encouraging purposeful free-market product innovation, all within realistic, achievable and cost-considerate supply chains. These RNS-empowered and fueled functional frameworks should be adaptable to emerging technologies, like environmentally clean and sustainable ingredient sourcing, supporting health and product-aligned outcomes.

### C. RNS AI-enabled Product Development Models:

a) “Good”, “Better” and “Best” Frameworks: Create tiered product development models:
   i. Good: Basic nutritional, general health products which meet minimum target health standards for general health, risk reduction, practical removal of toxins or chemicals.
   ii. Better: Enhanced products with functional ingredients (e.g., probiotics, omega-3s); transparency of health claims and intentional inclusion of beneficial ingredients.
   iii. Best: Tailored, purpose-aligned products; high quality nutritional properties; user-aligned health benefits. (e.g., cancer compliant, heart disease proficient, health-driven).

### D. RNS AI Consumer-enabling Engagement:

a) Education and Transparency: Engage consumers thru Relevant Nutrition Scores (RNS) AI and insights; align relevant product innovation to consumer-responsive, and purposeful health outcomes (clear labeling and nutrition claims which enable consumers to understand).
b) Leverage IRX FOODSCORE Platform: Aligns product innovations, formulations and purpose with consumer needs and health priorities, subject to health standards and claims.
c) Utilize RNS AI Ecosystem: Educates consumers about specific brand and product health advantages, product purposes, clean ingredient sourcing, safe packaging, clear health claims, and functional foods relevancy through product transparency; RNS AI certification platform. This will all build trust and encourage healthier dietary choices within target consumer bases.
d) Feedback Mechanisms: Establish RNS AI enabled channels for target consumer feedback loops, necessary to continuously improve purposeful product offerings based on use and relevancy preferences, priorities and objective health outcomes.

### E. RNS AI Continuous Evaluation in Support of Innovation:

a) Incorporate RNS AI Monitoring and Adaptation: Implement continuous evaluation processes necessary to assess the realized impacts of new products on objective general or specific health outcomes and environmental sustainability. This could involve collaborative care and integrated health initiatives with healthcare partners and providers, in order to assess the actualized effectiveness of “food as medicine” initiatives; supports product innovation and purpose claims within a positive, productive and purposeful functional feedback loop.
b) RNS-AI Supported Scalability Assessments: Regularly evaluate scalability and product feasibility of innovations to ensure they can be adopted widely across different regions and cultures without compromising product quality or safety.

IRX FOODSCORE frameworks, IRX AI and RNS AI-enabled SaaS and serviceable platforms, empower product innovators, brand drivers and manufacturers to reactively reformulate or proactively formulate powerful products, which are innovated through integrated and actionable health transparency. By integrating these components into a cohesive framework, product manufacturers can drive forward-looking innovations in global food systems, not only enhancing public health, but promoting sustainability and resilience in food production. This health attribute-applied and aligned approach will empower consumer packaged goods (CPG) manufacturers to innovate towards healthier standards, while mitigating known or misunderstood risks associated with product contaminants, dietary deficiencies and chronic health conditions.

(20) Healing and Recovery Protocols: RNS AI enables intentional, functional, situational and purposeful nutrition in support of the crucial healing and recovery protocols, and as an integral part of integrated medical care processes. Critically, by incorporating intentional nutritional strategies, healthcare providers can enhance healing processes and outcomes, empowering impaired individuals and at-risk patients to make informed, transparent and participatory decisions about personal healing and recovery. By doing so, this proactive nutrition approach (prescriptive dieting) promotes positive, empowered outcomes and improved recovery.

### A. RNS AI Aligns Nutrition as a Foundational Component of Care:

a) Nutrition is no longer an afterthought in medical treatment, but a fundamental aspect of holistic patient care. When integrated into healthcare protocols, purposeful nutrition serves as an equal complement to traditional treatments, medications, and care plans, seamlessly supported by RNS AI tools and technologies. This approach recognizes that planned, proper and purposeful nutrition is essential for (including but not limited to):
   i. Promoting healing, injury recovery and tissue repair
   ii. Supporting immune function and aligned antibodies
   iii. Managing chronic impairments and specific diseases
   iv. Improving overall and optimized health outcomes
B. RNS AI Supports Healing and Recovery:
   a) Nutrition plays a vital role in the body's ability to heal and recover from illness, injury, or surgery. Key nutritional strategies include:
      i. Optimizing quality and sources of protein intake
         a. RNS AI suggests quality sourced protein consumption to support tissue repair.
         b. RNS AI recommends lean meats, fish, legumes, and dairy products.
      ii. Optimizing essential nutrients based upon RNS AI assessed nutritional impacts on user-aligned, health-specific issues, treatments and recovery, focusing on vitamins and minerals crucial for healing, such as (Examples, as coordinated collaboratively between medical care professionals and patients, with collaborative access to RNS AI tools):
         a. Vitamin C for collagen synthesis
         b. Vitamin A for immune function
         c. Zinc for wound healing
      iii. Managing inflammation naturally through purposeful nutrition, as identified through RNS AI coordinated user, patient and medical practitioner assessments:
         a. Incorporating the use of health issue responsive anti-inflammatory foods/nutrients.
         b. Recommending health-aligned omega-3 fatty acids, antioxidants & phytonutrients.
      iv. Managing post-operative nutrition through procedure and health-aligned purposeful nutrition planning and intentional dietary guideline drivers, to ensure positive outcomes:
         a. RNS AI implements specific nutritional protocols for user-aligned, medical practitioner or nutritionist assigned, RNS AI designed dietary drivers, necessary to optimize surgery-specific recovery and outcomes.
         b. RNS AI addresses user-aligned increased energy and nutrient needs during healing process, considering all health issues, impairments and opportunities.
         c. RNS AI utilizes purposeful, natural nutrition for pain reduction or swelling management support and a purposeful complement to ensure prescriptive or over-the-counter (OTC) medication efficacy and impacts (beneficial or adverse).
         d. RNS AI considers all available patient-aligned health histories and dietary issues.

### C. RNS AI Empowers Informed and Comprehensive Decision-Making:

a) RNS AI enables consumers, nutritionists, medical providers and patients to make proactive and purposeful nutrition-aligned health decisions, providing all parties and users:
   i. Transparency in Purposeful Nutritional Information
      a. RNS AI provides clear, evidence-based and patient-aligned nutritional insights and interactive support, side-effect implications & clean relevant product sourcing options.
      b. RNS AI explains rationale behind dietary recommendations and expected results.
      c. RNS AI offers detailed information about nutrient content, interactions and effects.
   ii. Published Medical Sourcing, Nutrition Education and Consultative Considerations
      a. RNS AI offers users timely and relevant nutritional education tailored to individual and medical provider health goals and objective outcomes.
      b. RNS AI explains the evidentiary connection between diet and specific health outcomes, recovery timing and lifestyle impacts.
      c. RNS AI provides practical strategies for implementing dietary changes.
   iii. SaaS-driven platform for collaborative goal-setting (patients and practitioners)
      a. RNS AI allows patients to establish realistic and purposeful nutritional objectives.
      b. RNS AI supports regular reviews and adjustments to goal-setting based on progress
      c. RNS AI encourages patients' involvement in their proactive care and recovery plan
         D. RNS AI Integrates Purposeful Nutrition into Medical Care Processes:
a) RNS AI supports ability to fully leverage the power of nutrition in healthcare, employing and encouraging several strategies to support successful healing, risks and recovery:
   i. Institutionalizing Interdisciplinary Collaboration to Support Health Objectives
      a. RNS AI fosters teamwork between dietitians, physicians, and other specialists.
      b. RNS AI ensures that purposeful nutrition is considered in all aspects of patient care.
      c. RNS AI aligns comprehensive care plans, integrating actionable nutritional needs.
   ii. Enables Persistent Dietary and Nutritional Impact(s) Monitoring and Adjustments
      a. RNS AI enables regular assessment of nutritional status and intervention efficacy.
      b. RNS AI adapts nutritional strategies based on patient response and changing needs.
      c. RNS AI utilizes nutritional technologies for tracking and analyzing dietary intake.
   iii. Addressing Barriers and Availability of Nutritional Care
      a. RNS AI identifies and defines how to overcome obstacles to proper nutritioning.
      b. RNS AI provides dynamic, interactive and collaborative support for patients facing personal obstacles, dietary challenges, side-effect mitigation & drug interaction issues.
      c. RNS AI ensures access to nutritional resources and opportunistic interventions.

By embracing these principles, RNS AI empowers healthcare providers with the tools and technologies necessary to harness and optimize the power and potential of purposeful nutrition necessary to enhance healing, support recovery, and to improve overall patient outcomes. This integrated health approach not only addresses immediate health concerns, but also promotes long-term wellness and empowers individuals to take an active role in their own health journeys.

(21) Treatment Protocols and Pharmaceutical Efficacies: Treatment protocols and pharmaceutical efficacies are crucial aspects of modern healthcare that can be significantly influenced, facilitated or exacerbated by nutrition. RNS AI takes these interactions into account; providing personalized recommendations to optimize care outcomes. A treatment or treatment protocol encompasses any planned, standardized, or experimental intervention, procedure, or regimen medical, cellular, genetic, pharmacological, nutritional, dietary, behavioral, or otherwise intended to prevent, manage, or cure a disease or medical condition, including but not limited to medications, surgeries, radiation treatments, gene therapies, cell- and tissue-based therapies such as stem cell and regenerative medicines, biologics, behavioral health strategies, and medical devices.

### A. Treatment Protocols and Nutrition:

Treatment protocols are standardized approaches to managing specific health conditions and there are innumerable studies investigating how a patient's nutrition and dietary choices enhance or hinder the effectiveness of protocols; Simple, non-limiting examples considered by RNS AI:
a) Malnutrition and Treatment Outcomes: Severe energy or protein malnutrition reduce enzyme levels involved in drug metabolism; may alter the effectiveness of treatments.
b) Dietary Composition: The balance of macronutrients in a patient's diet can influence drug action and efficacy. For example, high-protein diets have been shown to increase drug oxidation rates compared to high-fat or high-carbohydrate diets.
c) Nutrient-Specific Interactions: Certain nutrients can directly impact the effectiveness of specific treatments. For instance, in Parkinson's disease patients, a high-protein diet can inhibit the transport of levodopa across the blood-brain barrier, reducing its efficacy.

### B. Pharmaceutical Efficacies and Nutrition:

The effectiveness and efficacies of medications can be significantly affected by a patient's diet and nutrition. RNS AI considers these interactions to provide tailored recommendations relevant to the user. Below are a few non-limiting examples of how nutrition can enhance pharmaceutical, prescriptive or over-the-counter (OTC) medications and resulting efficacies:
a) Absorption: Certain foods can increase or decrease the absorption of medications into the bloodstream. RNS AI advises on optimal timing of meals and medications to maximize absorption.
b) Metabolism: Dietary choices can affect how quickly the body processes and breaks down drugs, potentially altering their duration of action.
c) Excretion: Nutrition plays a role in how quickly medications are eliminated from the body, which RNS AI considers when making recommendations.

### C. Specific Nutrient-Medication Interactions:

As stated previously, there are numerous studies investigating nutrition and medication interactions. There are new studies published every day in this field, and they are all consolidated and tracked by RNS AI. Here are a few non-limiting examples:
a) Calcium Interactions
   i. Calcium can decrease absorption of tetracycline antibiotics.
   ii. Calcium carbonate (antacids) may decrease absorption of iron, zinc, magnesium, and fluoride.
b) Vitamin K Interactions
   i. High vitamin K intake (from green leafy vegetables) can reduce the effectiveness of warfarin, a blood thinner.
c) Grapefruit Interactions
   i. Grapefruit juice can interfere with blood pressure medicines, organ transplant medicines, and cholesterol-lowering drugs by affecting their metabolism.
d) Tyramine Interactions
   i. Foods high in tyramine (aged cheeses, processed meats, some wines) can cause dangerous blood pressure spikes in people taking MAO inhibitors.
e) B Vitamin Interactions
   i. Metformin may decrease absorption of vitamin B12.
   ii. Long-term use of proton pump inhibitors (PPIs); possible vitamin B12 deficiency.
f) Mineral Interactions
   i. Diuretics can cause loss of potassium, magnesium, and zinc.
   ii. Antibiotics like ciprofloxacin can decrease zinc absorption.
g) Iron Interactions
   i. PPIs and antacids may reduce iron absorption.
h) Protein Interactions
   i. High-protein diets may affect warfarin efficacy by raising albumin levels or cytochrome P450 activity.
i) Alcohol Interactions
   i. Alcohol can interfere with the metabolism of many medications, potentially increasing their effects or duration.
j) Vitamin C Interactions
   i. Vitamin C deficiency can decrease the activity of drug-metabolizing enzymes, especially in older adults.

These interactions highlight the importance of discussing both prescription and over-the-counter medications, as well as dietary habits, with healthcare providers not only to ensure safe and effective treatment, but also to optimize the benefits, efficacies and health outcomes.

### D. RNS AI and Optimizing Treatment Efficacy:

RNS AI integrates knowledge of treatment protocols and pharmaceutical efficacies with individual patient data to provide personalized or target population recommendations:
a) Chronic Condition Management: For chronic health conditions including but not limited to diabetes, hypertension, cancer, and heart disease, RNS AI suggests dietary choices that can enhance medication effectiveness.
b) Nutrient Timing: RNS AI provides guidance on when to consume specific nutrients in relation to medication schedules to optimize absorption and efficacy.
c) Dietary Adjustments: Based on a patient's medication regimen, RNS AI recommends increasing or decreasing intake of certain foods or nutrients to support treatment efficacy
d) Supplement Recommendations: In cases where medications may deplete certain nutrients, RNS AI suggests appropriate supplementation to maintain overall health and treatment effectiveness.
e) Monitoring and Adjustment: RNS AI continuously analyzes user data to detect potential nutrient-medication interactions and adjusts recommendations accordingly.

By considering the complex interactions between nutrition, treatment protocols, pharmaceutical and medicinal efficacies, RNS AI aims to optimize health outcomes and to improve the overall effectiveness and outcomes of medical interventions.

E. Cancer and Nutrition—Cancer as a non-limiting example of Chronic Conditions and their interactions with Nutrition: Cancer treatments and their interactions with nutrition are complex and multifaceted. The most common cancer treatments include chemotherapy, radiation therapy, and surgery, often used in combination. RNS AI considers these treatments and their nutritional implications when providing recommendations.

### a) Chemotherapy and Nutrition:

Chemotherapy uses powerful drugs to kill fast-growing cancer cells. However, it can also affect healthy cells, leading to various side effects that impact nutrition:
i. Appetite Changes: Chemotherapy can cause nausea, vomiting, and loss of appetite. RNS AI suggests foods that are easy to digest and nutrient-dense to maintain adequate nutrition during treatment
ii. Taste Alterations: Many patients experience changes in taste perception. RNS AI recommends flavor-enhancing strategies and alternative food choices to ensure continued nutrient intake.
iii. Nutrient Absorption: Some chemotherapy drugs can affect the gut lining, impacting nutrient absorption. RNS AI may suggest specific nutrients or supplements to counteract these effects.
iv. Drug-Nutrient Interactions: Certain foods can interact with chemotherapy drugs, affecting their efficacy. For example, grapefruit can interfere with the metabolism of some medications. RNS AI alerts users to potential interactions and suggests appropriate dietary adjustments.

### b) Radiation Therapy and Nutrition:

Radiation therapy uses high-energy beams to kill cancer cells. Nutritional considerations often include:
i. Localized Effects: Depending on the treatment area, radiation can cause localized side effects like mouth sores or digestive issues. RNS AI provides targeted nutritional strategies to manage these symptoms.
ii. Increased Nutritional Needs: Radiation can increase the body's energy and nutrient requirements. RNS AI recommends increased intake of proteins, healthy fats, and micronutrients to support healing and maintain strength.
iii. Frequent Hydration: Proper hydration is crucial during radiation therapy. RNS AI emphasizes the importance of fluid intake and suggests hydrating foods.

### c) Surgery and Nutrition:

Surgical interventions for cancer often require specific nutritional support:
i. Pre-surgery Nutrition: RNS AI may recommend a high-protein, nutrient-dense diet before surgery to support healing and recovery.
ii. Post-surgery Recovery: After surgery, RNS AI suggests easily digestible foods and gradually reintroduces a balanced diet to support wound healing and prevent complications.

### d) Emerging Treatments and Nutrition:

Newer cancer treatments, such as immunotherapy and targeted therapy, also have nutritional implications:
i. Immunotherapy: This treatment boosts the body's natural defenses to fight cancer. RNS AI may recommend foods that support immune function, such as those rich in antioxidants and omega-3 fatty acids.
ii. Targeted Therapy: These drugs target specific genes or proteins in cancer cells. RNS AI considers potential side effects and drug-nutrient interactions when making dietary recommendations.

### e) Dietary Interventions in Cancer Treatment:

Recent research has shown promising results for specific dietary interventions during cancer treatment:
i. Ketogenic Diet: Some studies suggest that a ketogenic diet may enhance the effects of chemotherapy and radiation therapy in certain cancers
ii. Fasting and Caloric Restriction: Short-term fasting or caloric restriction before chemotherapy may protect normal cells while making cancer cells more vulnerable to treatment
iii. Plant-based Diets: Diets rich in plant-based proteins, fruits, and vegetables may help manage treatment side effects and potentially improve outcomes

In the example above, RNS AI integrates emerging published scientific and medical research into its recommendations, tailoring dietary advice to individual patient needs, treatment protocols, and by types of cancer, all user-aligned. By considering a complex interplay between nutrition, cancer treatments, and individual patient factors, RNS AI aims to optimize treatment efficacy, while minimizing side-effects and improving overall quality of life for cancer patients. It is anticipated that stem cells in all forms, including but not limited to cellular products, gene therapies, targeted, cloned, and monoclonal, as they become readily available, along with precision nutrition, will be employed by RNS AI as intended by integrating any available emerging published scientific and medical research into its recommendations, tailoring dietary advice to individual patient needs, and user-aligned treatment protocols. Precision nutrition is a field that provides tailored dietary recommendations and interventions, analyzing an individual's unique biological, lifestyle, and environmental factors, such as genetics, metabolic responses and microbiomes, with a goal to optimize health, prevent and treat diseases, and improve overall well-being by recognizing that a “one-size-fits-all” approach to nutrition is ineffective.

In summary, the RNS AI approach is extended and offered for any chronic health or disease state, as well as preventative measures, episodic issues or objective health outcomes, and in support of any user-aligned issues or opportunities, impairments, or health outcomes defined and desired. Scientific, biological and medical research, interactions and planned, purposeful, and proactive interactions and efficacy empower users to make intentional and transparent decisions, enabled by RNS AI. From actionable insights to truly integrated health, knowledge is now much more than just a score!

(22) Promoting Wellness, Disease Resilience and Natural Immunities: RNS AI enables the creation of purposeful nutrition that promotes user-aligned wellness, disease resilience, and natural immunities, supporting numerous benefits and opportunities across various demographics and life stages. This approach to nutrition can significantly impact overall health and quality of life, tailored to individual needs and circumstances.

### A. Benefits of Purposeful Nutrition:

a) Enabling Enhanced Immune Function (Examples, including but not limited to):
   i. A user-aligned, purposeful and nutrient-rich diet supports a robust immune system, helping the body defend against pathogens and reduces the risk of infections. RNS AI refinery identifies crucial roles that specific nutrients can play. (Simple examples below)
      a. Vitamin C: Found in fruits and vegetables; may reduce common cold duration.
      b. Vitamin D: From fatty fish, egg yolk, and fortified dairy products; Supports immune function and natural preventions.
      c. Zinc: Found in lean meats, poultry, and beans. Supports immune cell production.
      d. Selenium: Found in Brazil nuts, tuna, meats, some grains and vegetables; is a key antioxidant with studied cancer-fighting properties.
b) Improving Disease Resilience (Examples, including but not limited to):
   i. RNS AI enables purposeful nutrition necessary to prevent/manage chronic diseases:
      a. Cardiovascular health: Omega-3 fatty acids from sources like salmon and olive oil have anti-inflammatory properties that may reduce risk of heart disease.
      b. Diabetes management: Specific user-aligned diets can help to regulate blood sugar levels, reducing risk of type 2 diabetes.
      c. Cancer prevention: Antioxidant-rich foods help to reduce risk of certain cancers.
c) Mental Health and Cognitive Function (Examples, including but not limited to):
   i. RNS AI identified nutrition can play vital role in mental health/cognitive performance:
      a. Mood regulation: B vitamins, found in whole grains and leafy greens, support healthy brain function and can help reduce stress levels.
      b. Cognitive health: Omega-3 fatty acids and antioxidants may help improve cognitive function and to reduce the risk of age-related cognitive decline.
d) Physical Performance and Body Composition (Examples, including but not limited to):
   i. Proper nutrition supports physical health and body composition:
      a. Muscle maintenance: Quality protein intake is crucial for maintaining muscle mass, especially in older adults.
      b. Energy levels: Complex carbohydrates provide sustained energy over time, and metabolize efficiently to support active lifestyles of children or adults.

### B. Opportunities for Personalized Nutrition:

a) Age-Specific Approaches (Examples, including but not limited to):
   i. RNS AI supports nutritional needs that vary across life stages:
      a. Children and adolescents: A focus on purposeful nutrients essential for growth and development, such as calcium and iron are opportunistic. However, RNS AI enables specific diets aligned with prevention, chronic health issues, diseases, etc.
      b. Adults: Emphasize disease prevention and maintenance of optimal health or achievement of key health outcomes and objectives. This is commonly implemented as gamification within applications. Critically, RNS AI identifies specific health benefits and risks of user-aligned accidental or intentional diets.
      c. Older adults: RNS AI addresses age-related changes in nutrient absorption and increased protein needs, as well as age-related illnesses and medication protocols.
b) Lifestyle-Based Nutrition (Examples, including but not limited to):
   i. RNS AI tailors nutritional strategies to individual lifestyles:
      a. Athletes: RNS AI emphasizes calorie-dense and nutrition-intense foods and proper hydration for optimal performance, aligned around user-specific health issues or objectives including but not limited to athletics.
      b. Sedentary individuals: RNS AI facilitates a focus on portion control and nutrient-dense foods necessary to prevent weight gain and related health issues.
c) Cultural and Geographical Considerations (Examples, including but not limited to):
   i. RNS AI adapts relevant nutritional recommendations to cultural preferences and local food availability:
      a. Cultural Alignment: RNS AI incorporates traditional foods and cooking methods that align with cultural values, dietary needs, and relevant health-driven purpose.
      b. Cultural Sensitivities: RNS AI considers regional differences in food availability and nutritional deficiencies, necessary to ensure user-aligned health objectives.
d) Life Stage-Specific Nutrition (Examples, including but not limited to):
   i. RNS AI addresses unique nutritional needs during different life stages:
      a. Pregnancy and lactation: RNS AI emphasizes folate, iron, and omega-3 fatty acids for fetal development, also identifying specific nutritional needs of a mother.
      b. Menopause: RNS AI enables diets focusing on natural calcium and vitamin D for bone health, while addressing purposeful nutrition for other health objectives.
e) Addressing Food Supply Chain Issues (Examples, including but not limited to):
   i. RNS AI defines strategies to ensure access to safe, high quality nutritious foods:
      a. Promotes local and sustainable food production to improve food security.
      b. Educates consumers on making healthy choices within available food options.

### C. Implementing Purposeful Nutrition (See Prior Sections of Glossary for More Details):

a) RNS AI creates and enables effective purposeful nutrition strategies: (example points)
   i. Conduct thorough nutritional assessments to identify user-aligned and relevant needs, actionable options, general or health-specific preventative or proactive health objectives.
   ii. Develop personalized meal plans that incorporate a variety of clean, whole foods, including fruits, vegetables, whole grains, lean proteins and healthy fats, each and all necessary to ensure fulfillment of lifestyle, life stage, immunity, stated health outcomes.
   iii. Provide transparency and insights related to purposeful and relevant food sourcing, packaging and preparation (cooking methods), necessary to maximize nutrient retention.
   iv. Assess nutrition-dense dietary drivers necessary to support specific physical activities, aligned with user-relevant health concerns and opportunities, for optimal health benefits.
   v. Monitor purposeful user-aligned nutritional progress and adjust intentional nutritional plans as needed to address changes in health status, new concerns and revised goals.

In summary and by implementing purposeful nutrition and risk-averse strategies, RNS AI users can harness the power of proactive and intentional nutrition to enhance overall health outcomes. RNS AI supports building natural immunities and resilience against diseases (prevention), dietary responses to chronic ailments (impairments), and proactive support of situational or beneficial health outcomes through relevant foods, products and dietary drivers.

In this respect, before explaining at least one embodiment of the Systems and Methods for Artificial Intelligence (AI) Analyses and Scoring of Consumables in greater detail, it is to be understood that the design is not limited in its application to the details of construction and to the arrangement of the components set forth in the following description or illustrated in the drawings. The Systems and Methods for Artificial Intelligence Analyses and Scoring of Consumables is capable of other embodiments and of being practiced and carried out in various ways. Also, it is to be understood that the phraseology and terminology employed herein are for the purpose of description and should not be regarded as limiting.

### SUMMARY OF THE INVENTION

The preferred embodiment of the Systems and Methods for Artificial Intelligence Analyses and Scoring of Consumables is configured to generate an IRX FOODSCORE AI-catalyzed cyber-ecosystem capable of analyzing and scoring consumables using a general and generative AI environment.

The principle advantage of the Systems and Methods for Artificial Intelligence Analyses and Scoring of Consumables generated IRX FOODSCORE AI-catalyzed ecosystem is to enable all-encompassing user-aligned, relevant health impact assessments of consumable products.

Another advantage of using the Systems and Methods for Artificial Intelligence Analyses and Scoring of Consumables generated IRX FOODSCORE AI-catalyzed ecosystem is to enable significantly faster, more actionable access to all-encompassing product health impacts.

Another advantage of using the Systems and Methods for Artificial Intelligence Analyses and Scoring of Consumables generated IRX FOODSCORE AI-catalyzed ecosystem is to enable rapid user-relevant health assessments related to specific ingredient or product consumption.

Another advantage of using the Systems and Methods for Artificial Intelligence Analyses and Scoring of Consumables generated IRX FOODSCORE ecosystem is to enable simpler user-aligned access to product-actionable nutritional research, insights, benefits and risks.

Another advantage of using the Systems and Methods for Artificial Intelligence Analyses and Scoring of Consumables generated IRX FOODSCORE ecosystem, tools and technologies is to provide user-aligned, on-demand health impact evaluations related to specific products.

Another advantage of using the Systems and Methods for Artificial Intelligence Analyses and Scoring of Consumables generated IRX FOODSCORE ecosystem is to enable user-relevant and dynamic all-encompassing product comparisons related to health, nutrition and impacts.

Another advantage of using the Systems and Methods for Artificial Intelligence Analyses and Scoring of Consumables generated IRX FOODSCORE ecosystem, software and applications is to provide user-aligned, health impact evaluations related to planned, purposeful nutrition.

Another advantage of using the Systems and Methods for Artificial Intelligence Analyses and Scoring of Consumables generated IRX FOODSCORE ecosystem is to provide user-aligned, all-encompassing health risk assessment related to actual or planned consumable products.

Another advantage of using the Systems and Methods for Artificial Intelligence Analyses and Scoring of Consumables generated IRX FOODSCORE ecosystem is to provide user-aligned relevant dietary support related to stated health objectives, lifestyle, life stage and culture.

Another advantage of using the Systems and Methods for Artificial Intelligence Analyses and Scoring of Consumables generated IRX FOODSCORE ecosystem is to provide user-aligned and relevant, preventative or situational opportunities for purposeful nutrition mapping.

Another advantage of using the Systems and Methods for Artificial Intelligence Analyses and Scoring of Consumables generated IRX FOODSCORE ecosystem is to provide user-aligned, relevant objective-driven purposeful nutrition mapping to optimize health outcomes.

Another advantage of using the Systems and Methods for Artificial Intelligence Analyses and Scoring of Consumables generated IRX FOODSCORE ecosystem is to provide user-aligned, all-encompassing scientific analyses and actionable insights related to purposeful dieting.

Another advantage of using the Systems and Methods for Artificial Intelligence Analyses and Scoring of Consumables generated IRX FOODSCORE ecosystem is to provide user-aligned and relevant, validation or reconciliation, in support of necessary or objective dietary lifestyles.

Another advantage of using the Systems and Methods for Artificial Intelligence Analyses and Scoring of Consumables generated IRX FOODSCORE ecosystem is to provide user-relevant vantage points to health impacts for actual or planned products, recipes or formulations.

Another advantage of using the Systems and Methods for Artificial Intelligence Analyses and Scoring of Consumables generated IRX FOODSCORE ecosystem is to enable user-relevant and dynamic assessment of product formulations or recipes, substituting healthier ingredients.

Another advantage of using the Systems and Methods for Artificial Intelligence Analyses and Scoring of Consumables generated IRX FOODSCORE ecosystem is to enable user-relevant and requested validations of product health claims and compliance with specified standards.

Another advantage of using the Systems and Methods for Artificial Intelligence Analyses and Scoring of Consumables generated IRX FOODSCORE ecosystem is to enable user-relevant dietary product recommendations to support impairments, chronic conditions or diseases.

Another advantage of using the Systems and Methods for Artificial Intelligence Analyses and Scoring of Consumables generated IRX FOODSCORE ecosystem is to enable user-relevant and dynamic evaluations of consumable products or ingredients that drive at-risk populations.

Another advantage of using the Systems and Methods for Artificial Intelligence Analyses and Scoring of Consumables generated IRX FOODSCORE ecosystem is to enable user-relevant and dynamic health-relevant certifications of intentional ingredients or purposeful products.

Another advantage of using the Systems and Methods for Artificial Intelligence Analyses and Scoring of Consumables generated IRX FOODSCORE ecosystem is to enable user-relevant transparency to intentional or incidental nutrition impacts at times of trauma or treatment.

Another advantage of using the Systems and Methods for Artificial Intelligence Analyses and Scoring of Consumables generated IRX FOODSCORE ecosystem is to enable user-relevant and actional insights as to the nutritional impacts upon absorbency or efficacy of medications.

Another advantage of using the Systems and Methods for Artificial Intelligence Analyses and Scoring of Consumables generated IRX FOODSCORE ecosystem is to enable user-relevant challenges to generally accepted principles and practices related to food supply chains.

Another advantage of using the Systems and Methods for Artificial Intelligence Analyses and Scoring of Consumables generated IRX FOODSCORE ecosystem is to support user-relevant improvements to health attributes associated with products, packaging and preparation.

Another advantage of using the Systems and Methods for Artificial Intelligence Analyses and Scoring of Consumables generated IRX FOODSCORE ecosystem is to enable user-relevant innovation and formulation of purposeful products, inspired by intentional ingredients.

These together with other advantages of the Systems and Methods for Artificial Intelligence Analyses and Scoring of Consumables, along with the various features of novelty, which characterize the design are pointed out with particularity in the claims annexed to and forming a part of this disclosure. For a better understanding of the Systems and Methods for Artificial Intelligence Analyses and Scoring of Consumables its operating advantages and the specific objects attained by its uses, reference should be made to the accompanying drawings and descriptive matter in which there are illustrated the preferred and alternate embodiments of the Systems and Methods for Artificial Intelligence Analyses and Scoring of Consumables. There has thus been outlined, rather broadly, the more important features of the design in order that the detailed description thereof that follows may be better understood, and in order that the present contribution to the art may be better appreciated. There are additional features of the Systems and Methods for Artificial Intelligence Analyses and Scoring of Consumables that will be described hereinafter, and which will form the subject matter of the claims appended hereto.

The preferred embodiment of the Systems and Methods for Artificial Intelligence Analyses and Scoring of Consumables will enable, in accordance with the present invention, a systematic and methodological AI analysis and scoring of consumables, comprising an integration of three key components: proprietary disruptive methods, proprietary systems and proprietary software; structured for the purpose of comprehensively identifying, collecting and refining disparate data that is relevant to diverse, user-aligned nutritional objectives and health outcomes.

With regard to the system component, the present invention employs a uniquely constructed general and generative AI system as its cornerstone. This system delivers global, user-relevant, actionable health and applied functional nutrition insights through concatenated content, scores and grades, related to an animal's, person's or population's incidental or purposeful consumption of foods, or designated consumables. An all-encompassing AI-empowered ecosystem enables and fuels a dynamic, disparate data refinery which privately or publicly sources, extracts, transforms, loads (ETL), conditions and compiles actionable user-aligned and relevant health, opportunity or risk-related information. This seamlessly integrated ecosystem is achieved through deployment of defined tactical and strategic algorithms and data lakes, relationally realized by leveraging AI in all forms, and by utilizing machine learning (ML) and deep learning (DL) technologies. DL technologies include large language models (LLMs) which are advanced, deep learning models trained on vast text datasets to understand and generate human-like text for tasks like translation, summarization, and question answering. Altogether, this process is necessary and leveraged to identify, evolve and optimize intended actionabilities and applications of fresh knowledge put to work through transparent insights, scores and grades. And upon further query or inquiry, which access cloud-based repositories, this systematic environment enables instructive applications, software and services to interactively deliver easy-to-understand and apply health and nutrition benefits and risks, and corresponding comprehensive impacts of any and all food supply chain activities associated with intake of identified foods or consumables for any targeted user, consumer or defined population.

It is anticipated that self-evolving AI, as it becomes available, will be employed as intended by leveraging AI in all forms.

With regard to the software component, the present invention provides dynamic content access through vantage-point driven, user-empowered software, actionable applications and interactive services like SaaS (Software as a Service) or Interactive Generative AI advisory interfaces. With respect to user-aligned software and applications, individual users and user communities ranging from consumers and consumer-packaged goods (CPG) manufacturers to restauranteurs, nutritionists, retailers, health providers, medical professionals, sports activists, educators & academia, researchers & scientists, military personnel, and governmental agencies & legislators, can access user-aligned, dynamic query, opportunity and issue-relevant feedback loops. Moreover, the present invention software platforms provide for user and issue-aligned vantage point, responsive content including but not limited to relevant nutrition scores (RNS) & grades, influencers of impact insights, comparative product scoring, risk-relevant assessment scoring, health claims & compliance validations, absorption impacts & efficacy metrics, each and all available for any existing or prospective consumables, products, formulations, recipes, foods, beverages, pharmaceuticals or medications. These dynamic deliverables are cloud-enabled and made available for licensed access or download.

With regard to the methods component, the present invention uses unique methods, approaches and algorithms for defining, refining and assigning user-aligned benefits and risks associated with an intake of identified foods or other consumables, existing or hypothetical. This is realized through ongoing, all-encompassing analyses of user-aligned and product-relevant data elements including but not limited to product ingredient sourcing, production, processing, preservatives, packaging and preparation and uses, which altogether have impacts on health outcomes and efficacies, accidentally or purposefully, situationally or conditionally, positively or negatively, proactively or reactively, whether opt-in or imposed. Critically, the methods herein defined apply to all global populations and cultures. The invention's AI-enabled methods, and their resulting and corresponding algorithms deployed, provide for seamless sourcing of disparate datasets that are user-aligned, health or dietary-driven, and product pertinent, with the resulting methods embracing the integrated ecosystem above to leverage ever-evolving functional frameworks. This methodical approach persistently provides for delivery of actionable content, in whole or in part. And the integrated methods and systems align to provide for a seamlessly integrated ecosystem comprising of a systematic data refinery and a methodological algorithmic library, engaged by or on behalf of distinct users. Together, the invention's unique methods and systems provide value-added and actionable knowledge repositories, newly discovered and applicable nutrition understandings, as well as refined applications for purposefully functional foods. Altogether, methods and algorithms of the invention facilitate and support proactive or reactive, informed decision-making by unique user-communities, each and all enabled though software, applications and services.

In summary, the present invention uniquely integrates, defines and aligns AI-empowered systems, methods and software necessary to deliver timely, actionable and relevant content to any user(s), reflecting all-encompassing analyses of influential disparate data and health impacts, of and pertaining to dietary influences, functional foods and defined consumables.

With respect to the above description then, it is to be realized that the optimum dimensional relationships for the parts of the Systems and Methods for Artificial Intelligence Analyses and Scoring of Consumables, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present design. Therefore, the foregoing is considered as illustrative only of the principles of the Systems and Methods for Artificial Intelligence Analyses and Scoring of Consumables. Further, since numerous modifications and changes will readily occur to those skilled in the art, it is not desired to limit the Systems and Methods for Artificial Intelligence Analyses and Scoring of Consumables to the exact construction and operation shown and described, and accordingly, all suitable modifications and equivalents may be resorted to falling within the scope of this application.

### BRIEF DESCRIPTION OF THE DRAWINGS

The accompanying drawings, which are incorporated in and form a part of this specification, illustrate embodiments of the Systems and Methods for General and Generative AI Analyses and Scoring of Consumables, and together with the description, serve to explain the principles of this application.

FIG. 1A shows how the Systems and Methods for Artificial Intelligence Analyses and Scoring of Consumables IRX Solution is the overlap of system, methods, and software;

FIG. 1B expands on FIG. 1A showing all services offered by the integrated solution;

FIG. 1C expands on FIG. 1B further defines each service enabled through nutritional technology;

FIG. 1D summarizes the IRX solution frameworks from data sourcing to RNS data deliverables;

FIG. 2 illustrates the user's choice to provide information;

FIG. 3A shows the initial question groups for the Consumer User Group;

FIG. 3B shows the initial question groups for the Consumer Brands & Products User Group;

FIG. 3C shows the initial question groups for the Retail Products & Private Label User Group;

FIG. 3D shows the initial question groups for the Nutritionists & Naturopaths User Group;

FIG. 3E shows the initial question groups for the Health & Medical Professions User Group;

FIG. 3F shows the initial question groups for the OTC/Pharmaceutical Brands User Group;

FIG. 3G shows the initial question groups for the Educational and Institutional User Group;

FIG. 3H shows the initial question groups for the Sports, Fitness and Trainers User Group;

FIG. 3I shows the initial question groups for the Cooks & Culinary Enthusiasts User Group;

FIG. 3J shows the initial question groups for the Researchers and Scientists User Group;

FIG. 3K shows the initial question groups for the Active Military and Veterans User Group;

FIG. 3L shows the initial question groups for the Governmental and Agency User Group;

FIG. 4 illustrates how RNS AI collects and stores Health Research;

FIG. 5 illustrates how RNS AI combines health research and user information;

FIG. 6 illustrates how RNS AI collects and stores product/ingredient information;

FIG. 7 illustrates how RNS AI creates a Complete Food Profile for given Input Product;

FIG. 8 illustrates how the Relevant Nutrition Score is calculated;

FIG. 9A shows that RNS can be used/accessed through the IRX FOODSCORE App on any device;

FIG. 9B introduces the initial dynamic opening options of the IRX App;

FIG. 9C details the three initial dynamic opening options of the IRX App;

FIG. 10A introduces the first initial option in the IRX App—Product Scan;

FIG. 10B shows how the user can utilize the IRX App to directly scan/input a product for information;

FIG. 10C shows how the IRX App can scan QR Code and other information codes to get information;

FIG. 10D defines two of the possible information codes—UPC and QR;

FIG. 10E illustrates key methods the IRX App utilizes to identify, collect, and capture consumable inputs;

FIG. 10F expands on FIG. 10E showing that information collected is digitized into actionable data;

FIG. 10G shows how the IRX App uses the scanned product information to return the user Relevant Nutrition Score;

FIG. 10H shows how the IRX App can score and compare products;

FIG. 11A introduces the second initial option in the IRX App—Rapid User Access;

FIG. 11B shows how the user can get a quick Relevant Nutrition Score by selecting a general baseline group;

FIG. 11C shows how a product can be scanned and scored referencing the baseline user group;

FIG. 11D illustrates how the same product can have different RNS values based on specific users;

FIG. 11E further expands on score differences for the same product between disparate users;

FIG. 12A introduces an example of the third initial option in the IRX App—User Input Access;

FIG. 12B shows 12 initial user groups with the first user (Consumer) highlighted as an example;

FIG. 12C shows the seven initial question groups for the first user group—Consumers;

FIG. 12D shows how the app can be accessed in multiple languages from any device;

FIG. 12E illustrates how the survey group questions will be initially presented in the IRX App;

FIG. 13A shows how each user group is defined and accessible through the IRX App on any device; and

FIG. 13B shows the 12 initial user groups, all in accordance with the preferred embodiments of the present invention.

### DETAILED DESCRIPTION OF THE PREFERRED EMBODIMENTS

As required, the detailed embodiments of the present Systems and Methods for Artificial Intelligence Analyses and Scoring of Consumables are disclosed herein, however, it is to be understood that the disclosed embodiments are merely exemplary of the design that may be embodied in various forms. Therefore, specific functional and structural details disclosed herein are not to be interpreted as limiting, but merely as basic for the claims and as a representative basis for teaching one skilled in the art to variously employ the present design in virtually any appropriately detailed structure as well as combination.

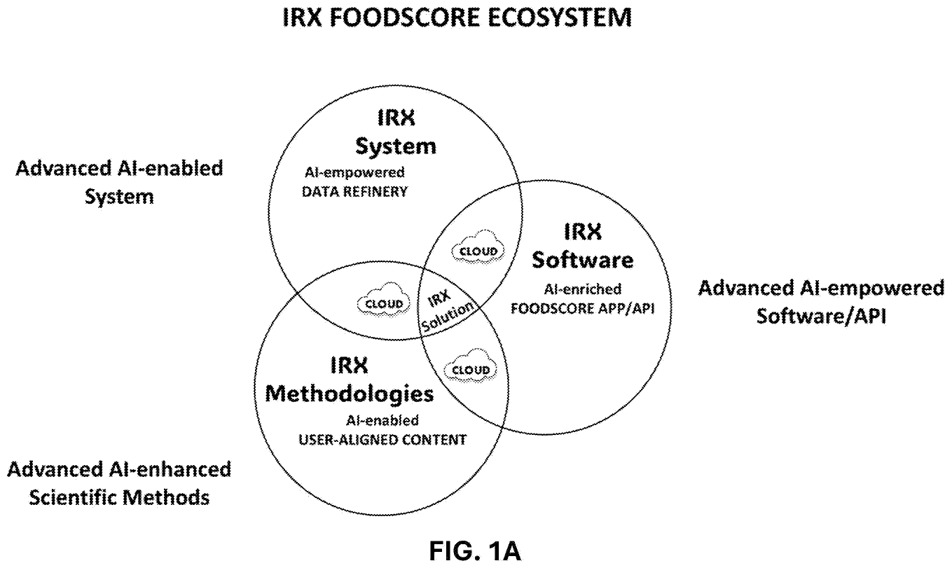

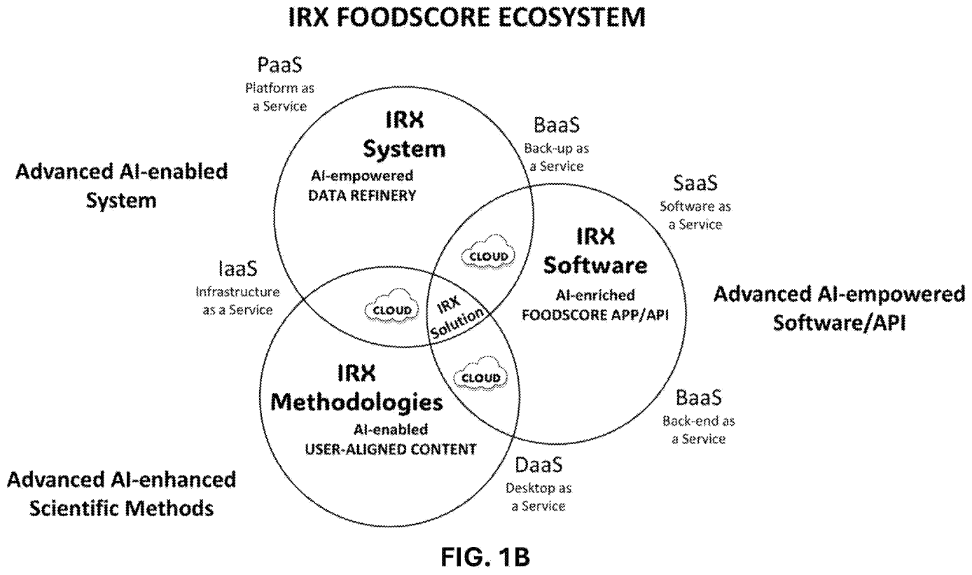

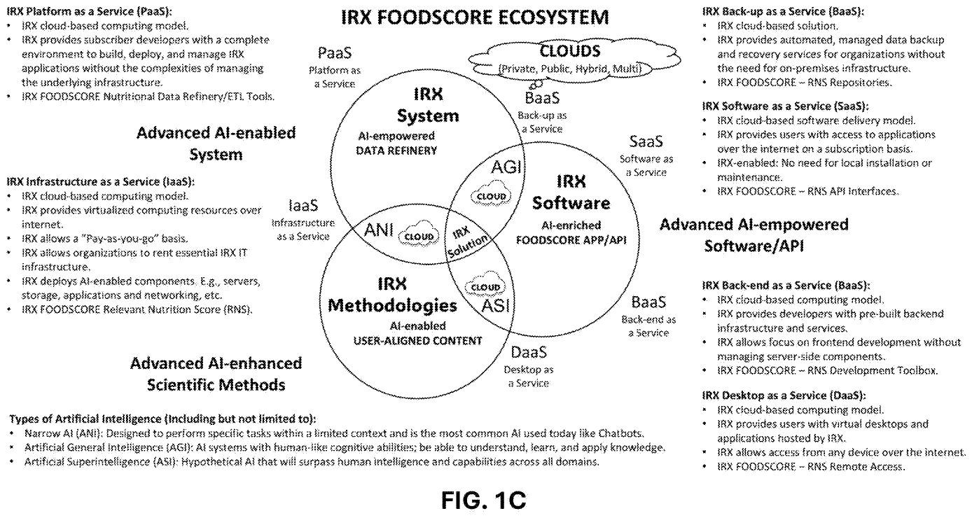

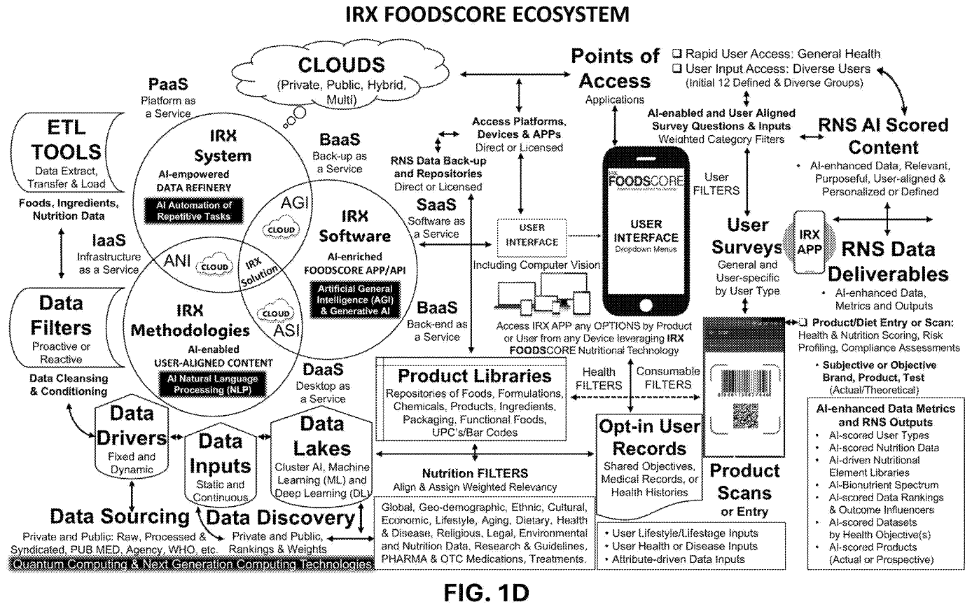

The IRX FOODSCORE solution incorporates three primary sections: IRXData-driven AI-Enabled EcoSystem (IRX System); IRXBusiness Methods (IRX Methods); and IRX Application Software (IRX Software) which are depicted in FIG. 1A. This integrated approach ensures user-aligned access to actionable and relevant health impacts, nutrition information and risks related to consumables, either directly or indirectly through several service options as depicted in FIG. 1B, including but not limited to: Infrastructure as a Service (IaaS), Platform as a Service (PaaS), Back-up as a Service (BaaS), Software as a Service (SaaS), Back-end as a Service (BaaS), Desktop as a Service (DaaS), collectively known as Anything as a Service (XaaS). FIG. 1C further breaks out each of these services into summarized bullet points, while more detailed descriptions of each can be found in the Glossary under (4) Advanced Algorithms. This figure also introduces the need for the solution to have Cloud-based access and support (Private, Public, Hybrid, and Multi).

Building upon the previous three figures, FIG. 1D represents a summarized and simplified depiction of the IRX FOODSCORE Solution framework. Showing the entire process from data sourcing and points-of-access through to and including user-aligned content, scores and other data deliverables and insights. Each item in the figure is supported by the remaining figures in this filing and our defined terms in the glossary. This system is designed to provide real-time, accessible commercial and consumer applications and services that can operate globally, across all languages and cultures, and at any age or stage of life, situational health or for objective health conditions and outcomes. The ETL tools referenced in FIG. 1D are designed to extract, transform, and load data from diverse and disparate sources into the IRX data refinery, automatically capturing detailed metadata for each data transaction, and tagging all scalable data objects to enable robust tracking, governance, and downstream analytics. Further expanding on FIG. 1D, treatments or a treatment protocol encompasses any planned, standardized, or experimental intervention, procedure, or regimen medical, cellular, genetic, pharmacological, nutritional, dietary, behavioral, or otherwise intended to prevent, manage, or cure a disease or medical condition, including but not limited to medications, surgeries, radiation treatments, gene therapies, cell- and tissue-based therapies such as stem cell and regenerative medicines, biologics, behavioral health strategies, and medical devices.

Commercially FIG. 1A—FIG. 1D, support the direct invention or creation of products with specific intent or purpose or alternatively scores health relevance, relevant nutrition score (RNS), or assesses risks of products, relevant risk score (RRS), against a stated objective, health issue or opportunity, or for a user-aligned conditional, situational or impaired dietary objective, any of which may be achieved through publication, prescription, subscription or licensing of the IRX FOODSCORE solution. The system is equally proficient at generating RRSs as by-products of generating RNSs, specifically directed to assess product-specific health-related risks and impacts associated with food and consumable products, and provide transparency to users. In this way, individuals, commercial business entities, medical providers, and governmental agencies can address RRS issues related to packaging, ingredient sourcing, storage, preparation practices, additives and preservatives, and other contaminants in an effort to mitigate health risk exposure to target populations and uses.

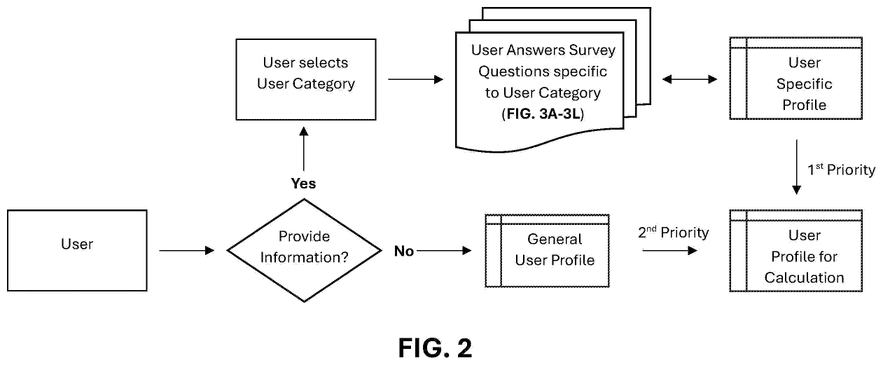

Referring now to FIG. 2, IRX Relevant Nutrition Scoring (RNS) starts with the user because each user is unique in their nutritional needs, goals, and motivations. For example, a 25-year-old female living in the United States with Breast Cancer will have different dietary needs than a 48-year-old male living in Japan with a familial history of heart disease. Given the need to assess each user individually, RNS AI asks the user to volunteer information about themselves dependent on which type of user they are. However, each user also has the option to not answer any questions if they simply wish to score a food item for a general/average individual. This initial process is mapped out in the diagram illustrated in FIG. 2.

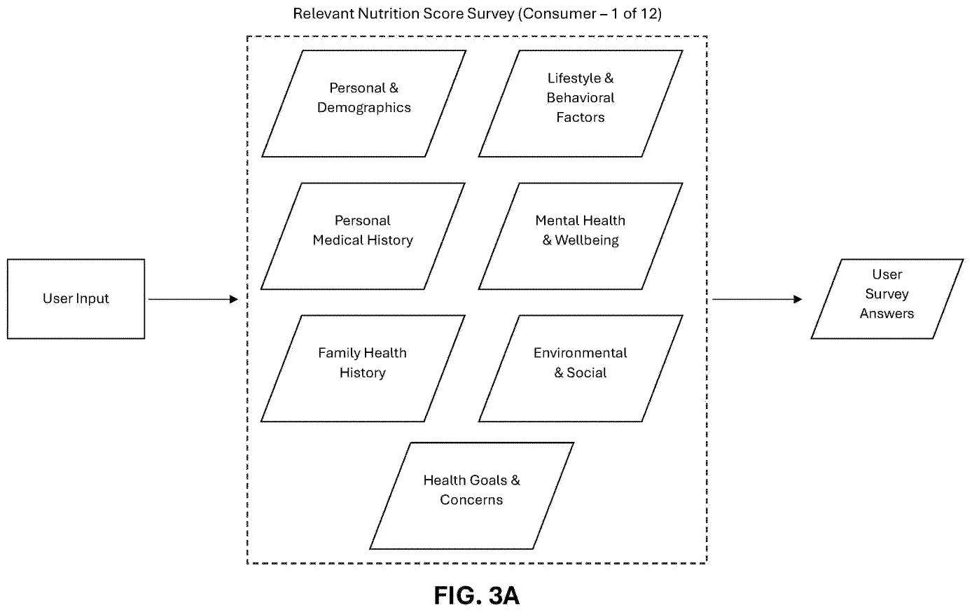

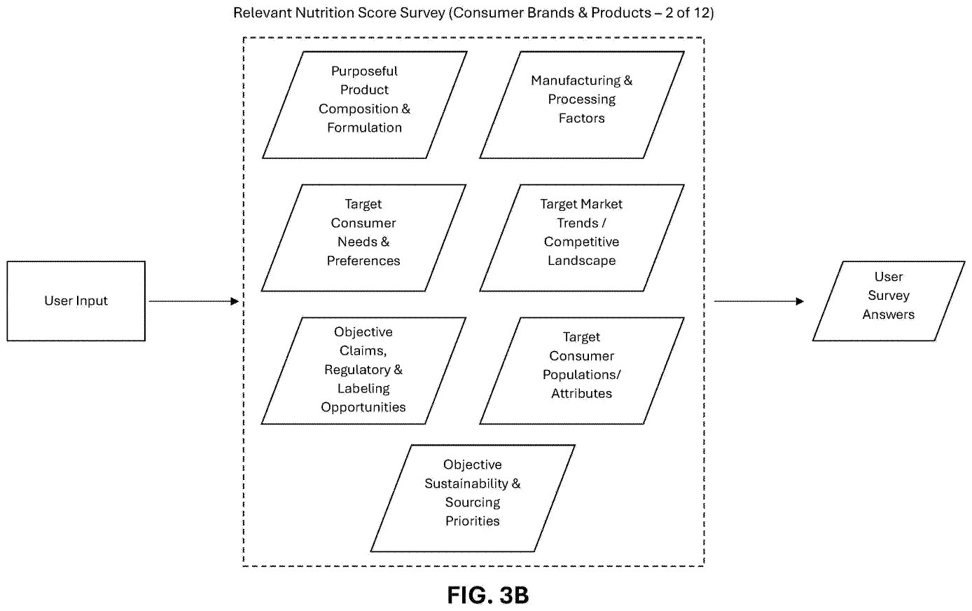

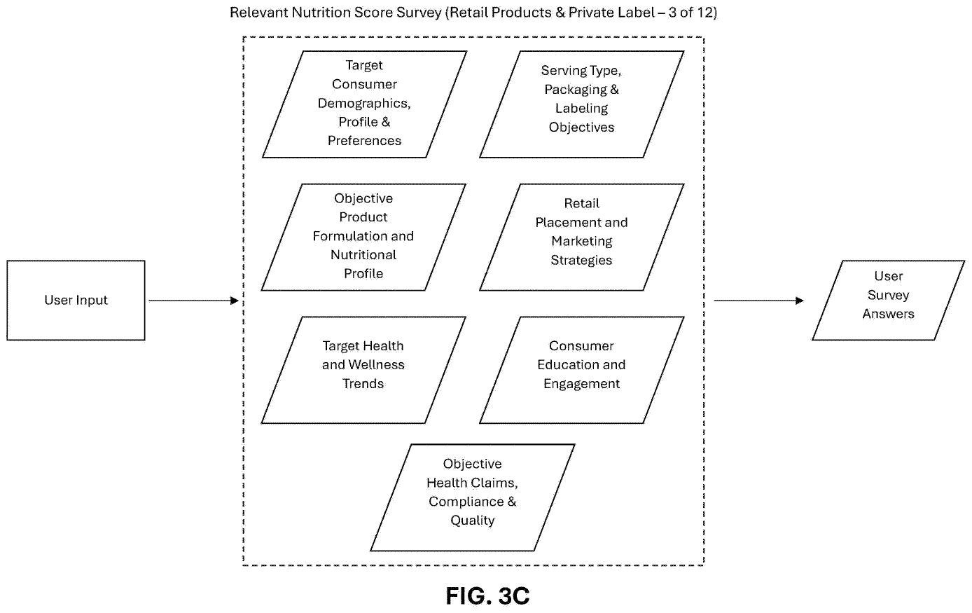

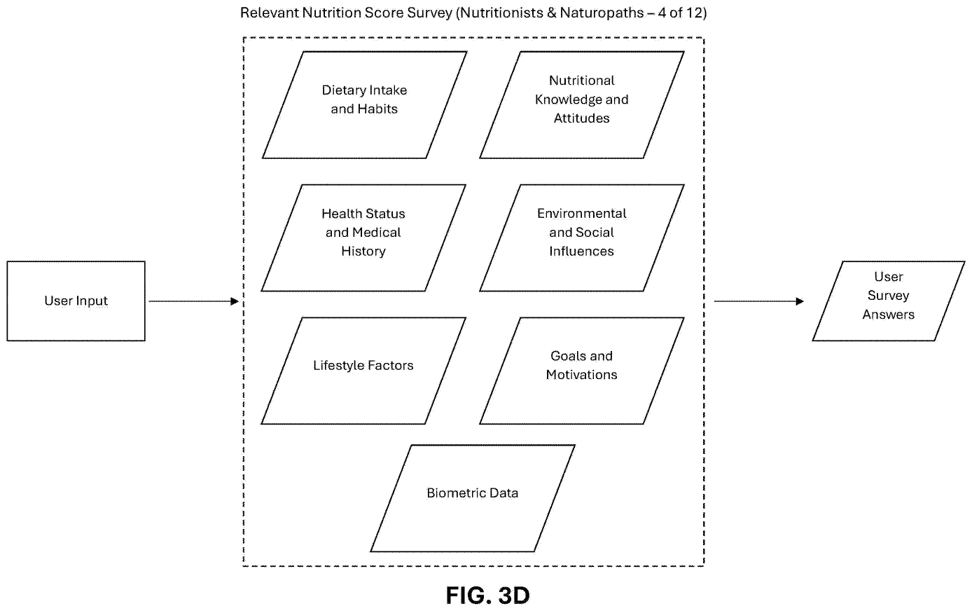

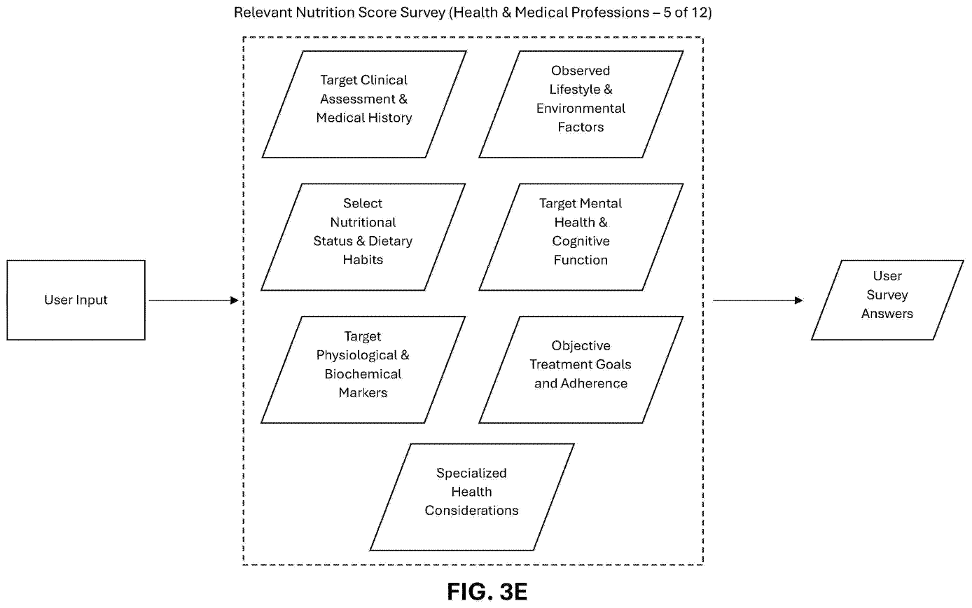

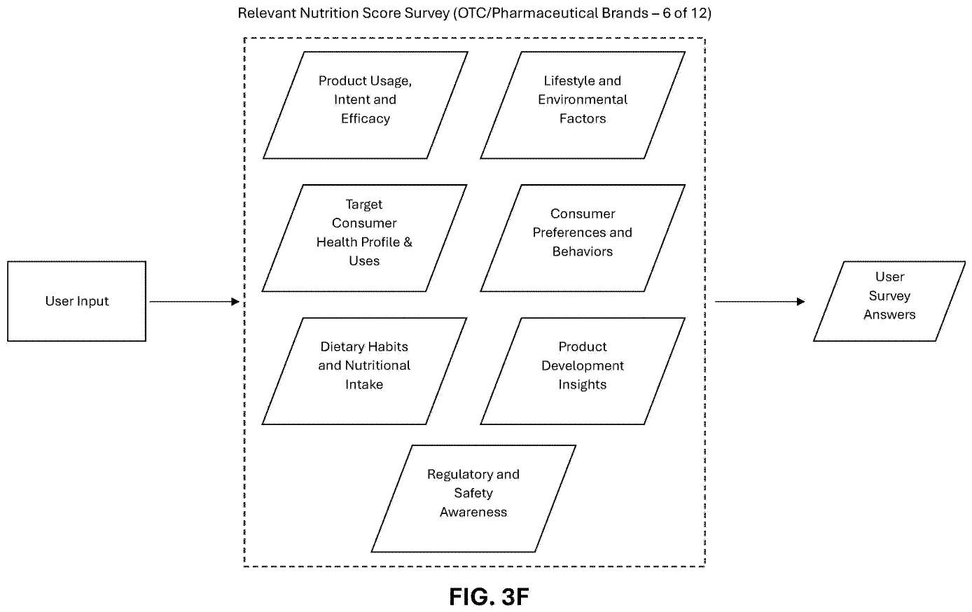

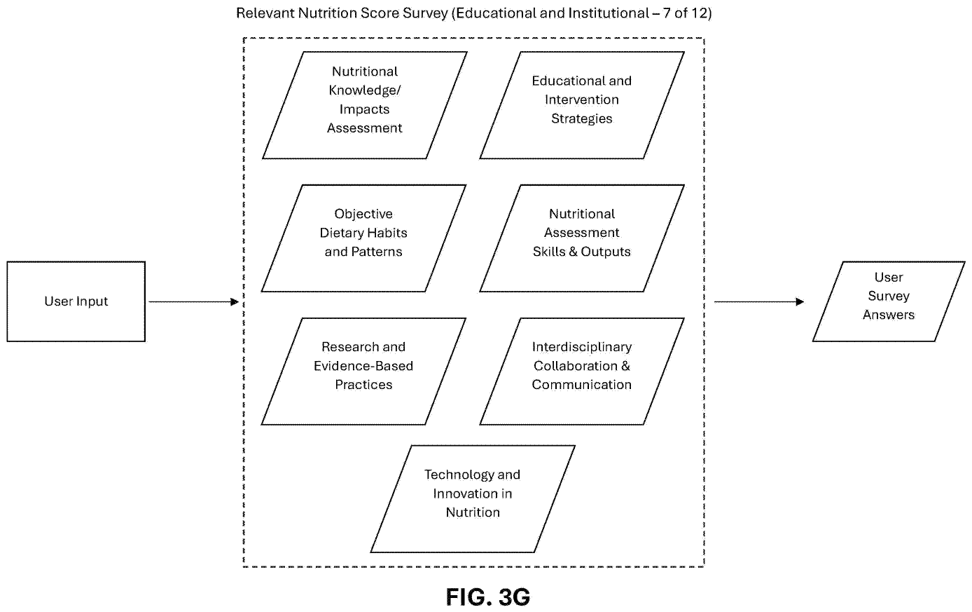

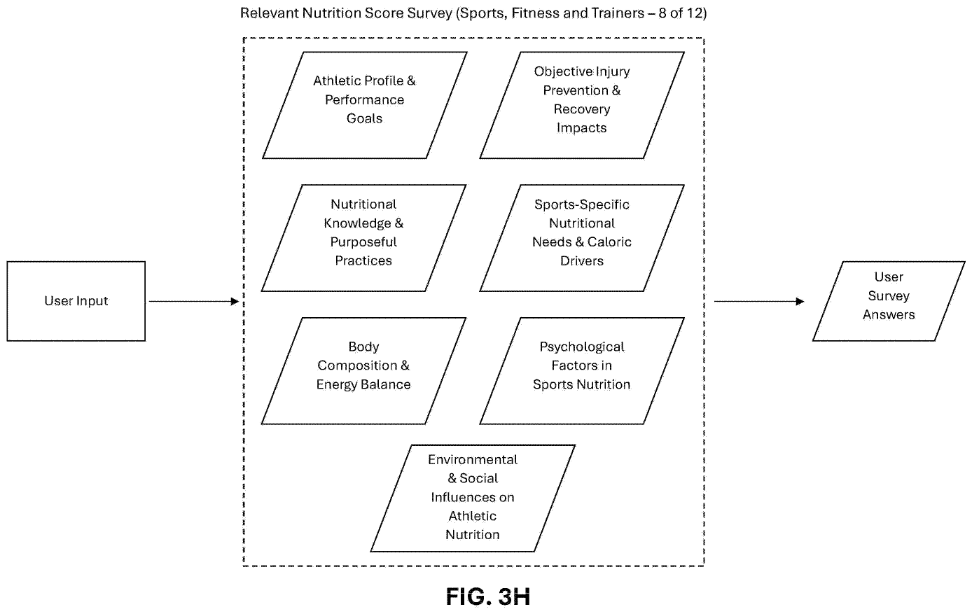

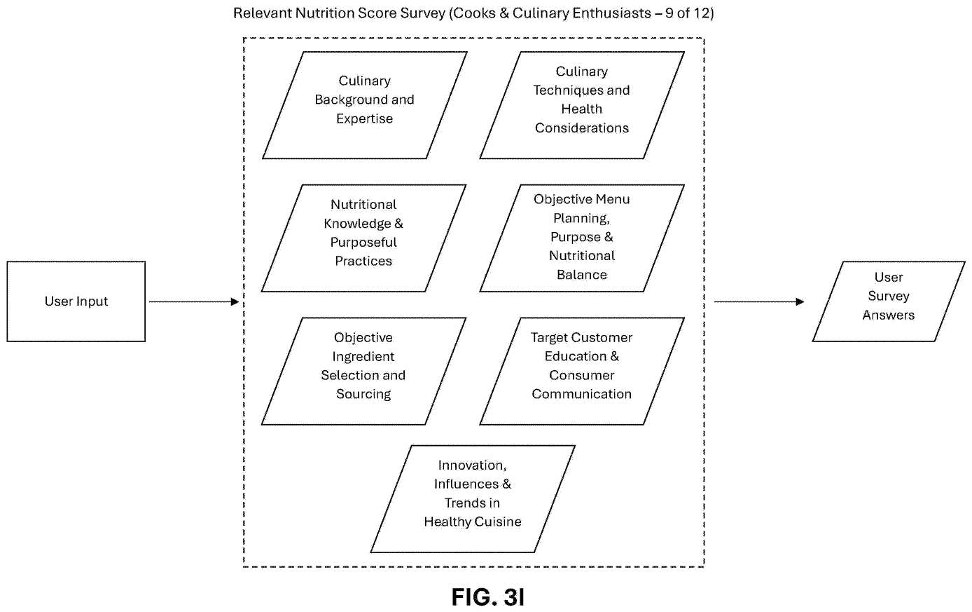

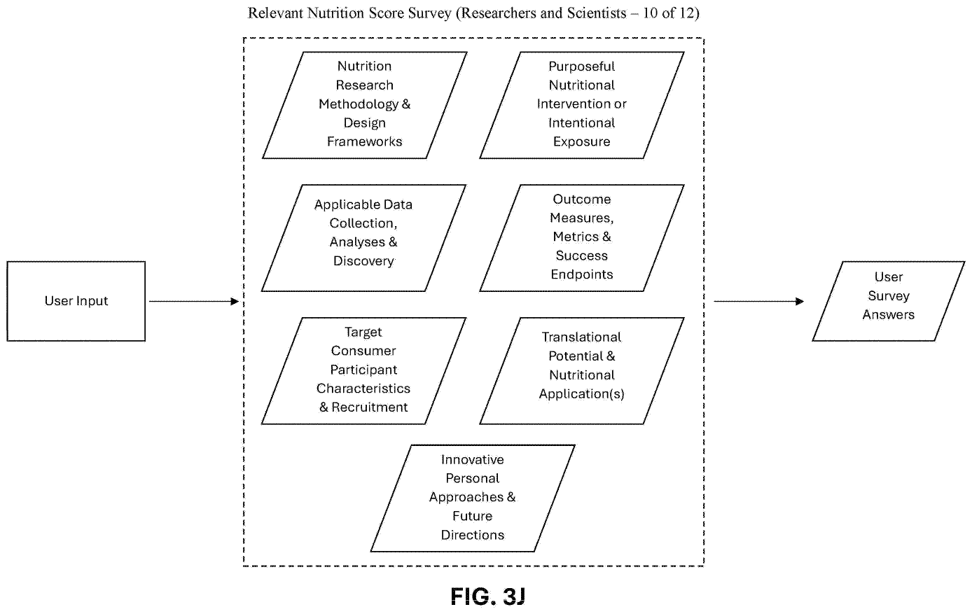

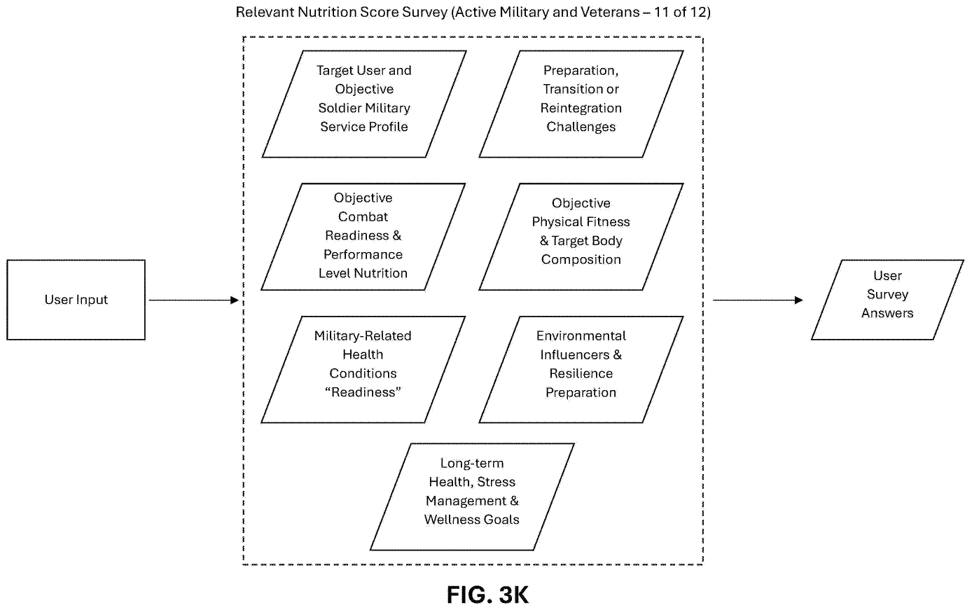

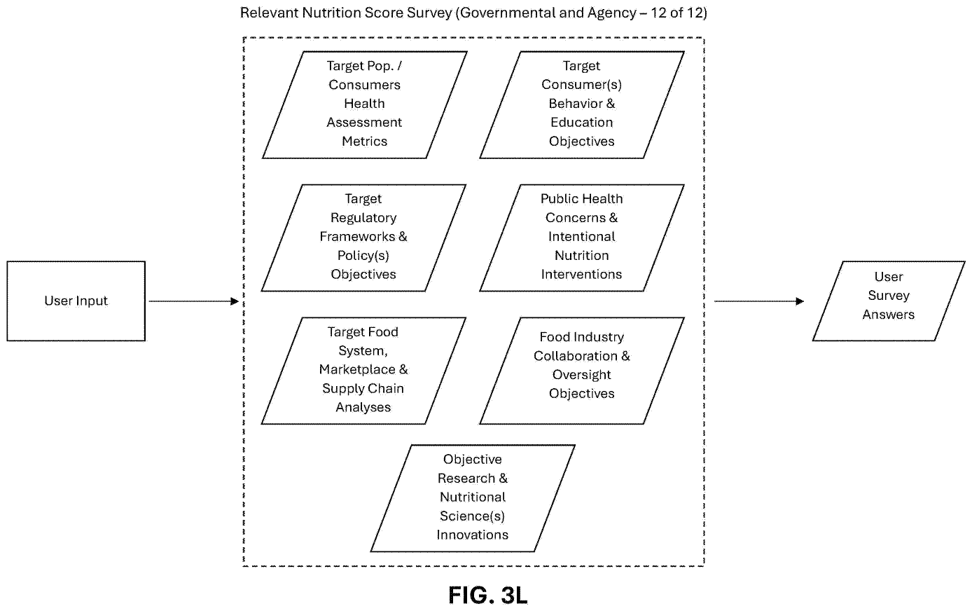

FIG. 3A through FIG. 3L show the 12 different Dynamic User Groups that are currently defined and subject to change as new research deems fit. The current user groups are: Consumer, Consumer Brands & Products, Retail Products & Private Label, Nutritionists & Naturopaths, Health & Medical Professions, OTC/Pharmaceutical Brands, Educational and Institutional, Sports, Fitness and Trainers, Cooks & Culinary Enthusiasts, Researchers and Scientists, Active Military and Veterans, and Governmental and Agency. Each user group contains its own set/group of questions dependent on the primary concerns for each group and these question groups are shown in each figure. Again, it should be noted that the user can choose to answer as many or as few questions as they want, with the understanding that the more questions answered will provide a more personalized User-Specific Profile.

Referring now to FIG. 3A there is illustrated the Relevant Nutrition Score Survey for Consumer (user 1 of 12).

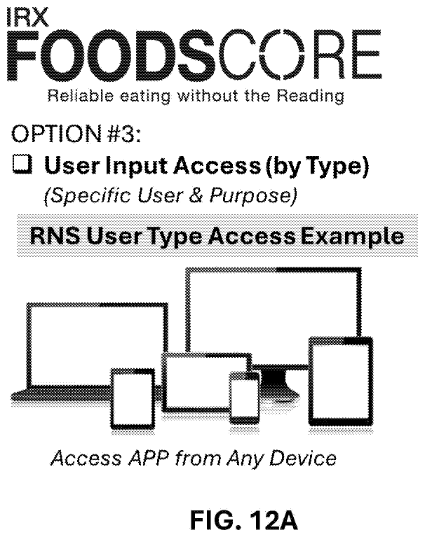

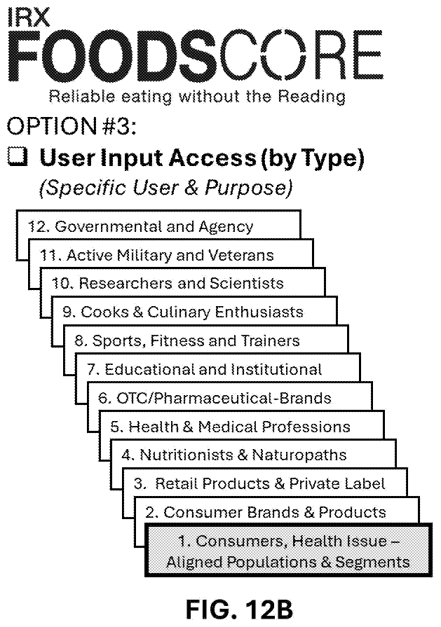

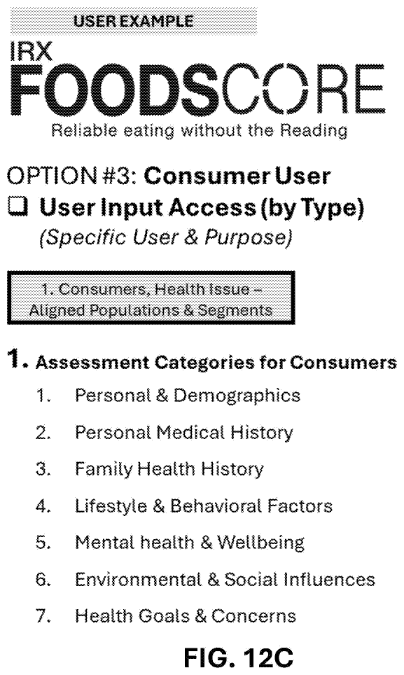

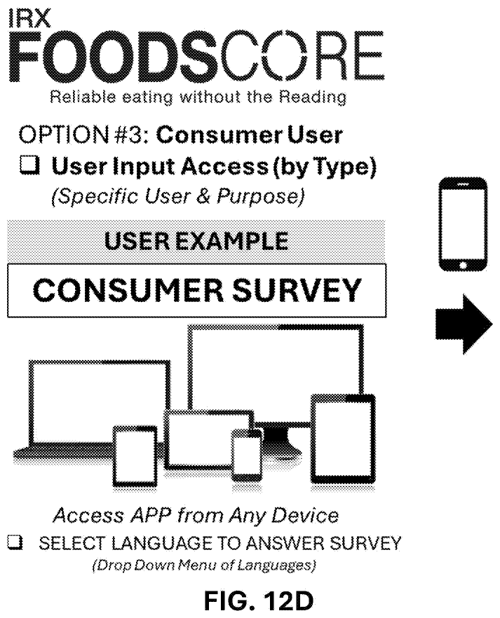

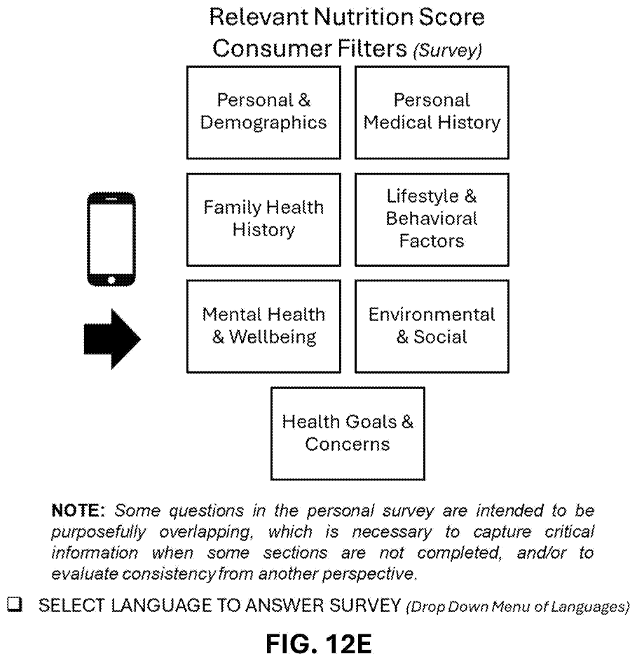

### 1. USER 1: RNS Consumer Apps: Consumer User Example (FIG. 12A-12E, FIG. 3A)

A. RNS Assessment Categories for Consumers: Opt-in Inputs, Disclosures and Objectives
   1. Personal & Demographics
   2. Personal Medical History
   3. Family Health History
   4. Lifestyle & Behavioral Factors
   5. Mental health & Wellbeing
   6. Environmental & Social Influences
   7. Health Goals & Concerns
B. RNS Food, Product or Brand Scores: IRX FOODSCORE—Consumer Scoring Options
   1. General Health & Nutrition Ratings, Scores, Grades, Insights
      a. Accepted Guideline(s) or Prevailing Standard(s): (e.g., US FDA; KETO Diet, etc.)
   2. Relevant Health & Nutrition Rating, Score, Grade and Insights
      a. Macro Health Objective, Affliction or Disease: (e.g., Triathlete or Cancer)
   3. Personal Health & Nutrition Rating, Score, Grade and Insights
      a. Micro Personal or Consumer Target-Specific (e.g., 18 YO Female w/Breast Cancer)
   4. Comparative Health & Nutrition (1, 2 or 3 Above); Comparative Scores, Grades and Insights
      a. Product Side-by Side Attributes; Product vs. Product Scores and Grades
C. RNS Consumer-aligned Actionable Insights: User Application(s) & Software Functionality
   1. Health-relevant Product(s) Finder
      a. Relevant Product, Score, Sourcing or Ranking
   2. Meal Planning/Target Nutrients
      a. Relevant Meal Ingredients, Nutrition and Purposeful Recipes
   3. Store-SCORE Relevant Shopping: Retailer-aligned Product Relevance (SKU's)
      a. Relevant Product Store Locator: General Health or Specific Health
   4. Environmental Impacts of User-Diet (existing or proposed)
      a. Known Environmental or Identified Food-related Factors (Supply Chain)
   5. Product, Packaging, Preservative & Preparational Practices (P5)
      a. Product-relevant P⁵ Factors & Impacts to Target Consumer (Weighted Contributions)
   6. Food-Medication Interaction(s): Over-the-Counter (OTC) or Prescription PHARMA
      a. Tolerances or Intolerances; Interactions, Efficacy & Absorbency Impacts
   7. Relevant Nutrition Advisor—interactive AI (e.g., Google Gemini Interactive Applications)
      a. Conversational AI—Consumer Nutrition Advisory Services and Suggestive Nutrition

Referring now to FIG. 3B there is illustrated the Relevant Nutrition Score Survey for Consumer (user 2 of 12).

### 2. USER 2: RNS Consumer Packaged Goods and Product Management User App(s) (FIG. 3B)

### A. RNS Assessment Categories for Consumer Brands & Product Users: Inputs & Objectives

1. Brand Certification, Purposeful Product Validation and Reformation Objectives
2. Target Consumer Needs & Preferences, Priorities, Preferred Traits and Impacts
3. Subjective or Objective Health Claims, Regulatory & Labeling Compliance & Risks
4. Manufacturing & Processing Factors by Type, Influence, Sustainability and Impacts
5. Target Market Trends/Competitive Landscape; Contrast Product Positioning Drivers
6. Target Consumer Populations and Attributes; Purposeful Product Formulaic Content
7. Sustainability & Sourcing Priorities; Product and Packaging Environmental Impacts

### B. RNS Food, Product or Brand Scores: IRX FOODSCORE—Brand and Product Options

1. Brand or Product(s)' General Health Application & Nutrition Rating, Score, Grade, Insights
   a. Accepted Guideline(s) or Prevailing Standard(s): (e.g., US FDA; Heart Healthy Diet)
2. Target Brand/Product Relevance to Health & Nutrition; Relevant Nutrition/Attribute Coding
   a. Macro Health Objective, Affliction or Disease: (e.g., Aerobic Sports or Diabetes)
3. Brand and Product-aligned Specific Health Prevention or Proactive Nutrition
   a. Micro Health or Consumer Target-Specific (e.g., Hispanic Males w/Prostate Cancer)
4. Purposeful Brand and Functional Product Comparative Health & Nutrition (1, 2 or 3 Above)
   a. Side-by Side Attributes; Brand vs. Brand or Products vs. Products Scores
   b. Brand and Product Risk Assessment & Ranking (Products, ingredients, health claims)

### C. RNS Brand/Product-aligned Actionable Insights: User App(s)-Software Functionality

1. Brand and Product Certification of Purposeful, Health-relevant Product Claims & Positioning
   a. Relevant Product Score, Rankings and Related Health Claims Risk Assessments
2. Branded or Productized Meal Planning/Target Nutrients (existing or hypothetical)
   a. Relevant Meal Ingredients, Nutrition and Purposeful Recipes
3. Store-SCORE Relevance: Brand-Retailer aligned Product Distribution Relevance (SKU's)
   a. Brand/Product-aligned Relevant Product Store Insights: General or Specific Health
4. Brand and Product-aligned Environmental Insights and Impacts (existing/proposed)
   a. Brand and Product-aligned Environmental or Food-related Factors (Supply Chain)
5. Brand and Product, Packaging, Preservative & Preparational Practices (P5)
   a. Product-relevant P⁵ Factors & Impacts to Target Consumer (Weighted Contributions)
6. Brand-aligned Medication Interaction(s): Over-the-Counter (OTC), Prescription PHARMA
   a. Tolerances or Intolerances; Interactions, Efficacy & Absorbency Impacts
7. Brand Relevant Nutrition Advisor—interactive AI (e.g., Google Gemini Interactive APPS)
   a. Conversational AI—Brand/Product Nutrition Advisory Services; Suggestive Nutrition

Referring now to FIG. 3C there is illustrated the Relevant Nutrition Score Survey for Consumer (user 3 of 12).

### 3. USER 3: RNS Retailer and Private Label Brand, Food and Product User App(s) (FIG. 3C).

### A. RNS Assessment Categories for Retail & Private Label Users: Inputs and Objectives

1. Target Shopper and Consumer Demographics, Profile & Preferences
2. Objective Product Formulations, Nutritional Profiles and Lifestyle Impacts
3. Retail and Private Label Target Market(s) Health and Wellness Trends
4. Serving Types, Packaging & Labeling Objectives, Claims and Disclosures
5. Retail Placement, Marketing Strategies and Nutritional Objectives
6. Consumer Education and Engagement Highlights in Support of Health Positions
7. Objective Health Claims, Governing Compliance and Nutritional Quality & Purpose

### B. RNS Retail Food, Product or Brand Scores: IRX FOODSCORE—Brand/Product Options

1. Retail Brand/Product General Health Application & Nutrition Rating, Score/Grade, Insights
   a. Accepted Guideline(s) or Prevailing Standard(s): (e.g., US FDA; Heart Healthy Diet)
2. Retailer-aligned Private Label or National Brand or Product Relevance to Health & Nutrition
   a. Macro Health Objective, Affliction or Disease: (e.g., Aerobic Sports or Diabetes)
3. Retailer Brands or Product-specific Health Prevention or Proactive Nutrition Ratings
   a. Micro Health or Consumer Target-Specific (e.g., Hispanic Males w/Prostate Cancer)
4. Retailer Brands and Products Comparative Health & Nutrition (1, 2 or 3 Above)
   a. Side-by Side Attributes; Brand vs. Brand or Products vs. Products Scores
   b. Brand and Product Risk Assessment & Ranking (Products, ingredients, health claims)

### C. RNS Retailer-aligned Actionable Insights: User App(s)-Software Functionality

1. Retailer Brand and Product Certification of Health-relevant Purpose and Positioning
   a. Relevant Product, Score Sourcing or Ranking
2. Retailer objective-driven Meal Planning/Target Nutrients
   a. Relevant Meal Ingredients, Nutrition and Purposeful Recipes
3. Store-SCORE Relevant Shopping: Retailer-aligned Product Relevance (SKU's)
   a. Relevant Product Store Locator: General Health or Specific Health
4. Retailer Brand and Product-aligned Environmental Insights and Impacts (existing/proposed)
   a. Known Environmental or Identified Food-related Factors (Retailer/Supply Chain)
5. Retailer-aligned Product, Packaging, Preservative & Preparational Practices (P5)
   a. Product-relevant P⁵ Factors & Impacts to Target Consumer (Weighted Contributions)
6. Retail Brand Medication Interaction(s): Over-the-Counter (OTC) or Prescription PHARMA
   a. Tolerances or Intolerances; Interactions, Efficacy & Absorbency Impacts
7. Retailer Relevant Nutrition Advisor—interactive AI (e.g., Google Gemini Interactive APP)
   a. Conversational AI—Retailer Nutrition Advisory Services and Optimized Nutrition

Referring now to FIG. 3D there is illustrated the Relevant Nutrition Score Survey for Consumer (user 4 of 12).

### 4. USER 4: RNS Nutritionist & Naturopath Food, Ingredient, Product User App(s) (FIG. 3D)

A. RNS Assessment Categories for Nutritionists/Naturopath Users: Inputs and Objectives
1. Dietary Intake and Habits
2. Health Status and Medical History
3. Lifestyle Factors
4. Nutritional Knowledge and Attitudes
5. Environmental and Social Influences
6. Goals and Motivations
7. Biometric Data

### B. RNS Food, Product or Brand Score: IRX FOODSCORE Nutritionist/Naturopath Options

1. General Food, Product Health and Purposeful Nutrition Ratings, Scores, Grades, Insights
   a. Specified Guideline(s) or Prevailing Standard(s): (e.g., US FDA; Diabetes)
2. Products and Purpose Relevant Health & Nutrition Ratings, Scores, Grades, and Insights
   a. Macro Nutrient Health Objectives, Efficacy or Interactions (e.g., Athlete or Diabetic)
3. Patient Health and Objective Dietary Nutrition Ratings, Scores, Grades and Insights
   a. Micro Personal or Consumer Patient-specific (e.g., 48 YO Male w/Prostate Cancer)
4. Objective Health and Nutrition (1, 2 or 3 Above); Dietary Scores, Grades and Insights
   a. Specified Nutrition-driven Purposeful Lifestyle and Recommended Product Scores

### C. RNS Nutritionist-aligned Actionable Insights: User App(s) & Software Functionality

1. Specified Lifestyle Life Stage or Designated Health State-relevant Product(s) Finder
   a. Nutrition-relevant Products, Scores, Sourcing or Ranking
2. Patient-aligned Meal Planning with Designated Nutrients, considering Health Objectives
   a. Relevant Meal Ingredients, Nutrition and Purposeful Recipes
3. Patient-specified Store-SCORE Relevant Shopping: Health-state Product Relevance (SKU's)
   a. Patient Condition-relevant Products Store Locator: General Health or Specific Health
4. Environmental Impacts of Patient-specified User-Diet (existing or proposed)
   a. Known Environmental or Identified Food-related Factors (Supply Chain)
5. Patient-aligned Product, Packaging, Preservative & Preparational Practices (P5)
   a. Product-relevant P⁵ Impacts of Specific Patient Treatments (Weighted Contributions)
6. Nutrition-Medication Interaction(s): Over-the-Counter (OTC) or Prescription PHARMA
   a. Nutrition Tolerances or Intolerances; Interactions, Efficacy & Absorbency Impacts
7. Relevant Nutrition Advisor—interactive AI (e.g., Google Gemini Interactive Applications)
   a. Conversational AI—Nutritionist Advisory Services and Patient-aligned Nutrition

Referring now to FIG. 3E there is illustrated the Relevant Nutrition Score Survey for Consumer (user 5 of 12).

### 5. USER 5: RNS Integrated Health/Medical Professional Patient-aligned User App(s) (FIG. 3E).

### A. RNS Assessment Categories for Health/Medical Professional Users: Inputs & Objectives

1. Target Clinical Assessment & Medical History
2. Select Nutritional Status & Dietary Habits
3. Target Physiological & Biochemical Markers
4. Observed Lifestyle & Environmental Factors
5. Target Mental Health & Cognitive Function
6. Objective Treatment Goals and Adherence
7. Specialized Health Considerations

### B. RNS Food, Nutrition/Products Scores: IRX FOODSCORE—Medical Professional Options

1. Patient Nutrition, General Food, Integrated Health Impacts, Ratings, Scores, Grades, Insights
   a. Patient-specified Guideline(s) or Prevailing Standard(s): (e.g., US FDA; Diabetes)
2. Patient-aligned, Purposeful Product and Relevant Nutrition Ratings, Scores, Grades, Insights
   a. Macro Nutrient Health Objectives, Efficacy or Interactions (e.g., Heart Disease)
3. Patient Personalized, Integrated Health & Dietary Nutrition Ratings, Scores, Grades, Insights
   a. Micro Personal or Consumer Patient-specific (e.g., 50 YO Male w/Crohn's Disease)
4. Treatment-related Health & Nutrition (1, 2 or 3 Above); Dietary Scores, Grades and Insights
   a. Patient-aligned Nutrition-driven Purposeful Lifestyle; Recommended Products Scores

### C. RNS Medical Practitioner-aligned Actionable Insights: User App(s) & Software

1. Specified Patient Lifestyle, Life Stage or Designated Health State-relevant Product(s) Finder
   a. Nutrition-relevant Products, Scores, Sourcing or Ranking
2. Patient-aligned Meal Planning with Designated Nutrients, considering Health Objectives
   a. Relevant Meal Ingredients, Intentional Nutrition and Purposeful Recipes
3. Patient-specified Store-SCORE Relevant Shopping: Health-state Product Relevance (SKU's)
   a. Patient Condition-relevant Products Store Locator: General Health or Specific Health
4. Environmental Impacts of Patient-specified User-Diet (existing or proposed)
   a. Known Environmental or Identified Food-related Factors (Supply Chain)
5. Patient-aligned Product, Packaging, Preservative & Preparational Practices (P5)
   a. Product-relevant P⁵ Impacts of Specific Patient Treatments (Weighted Contributions)
6. Nutrition-Medication Interaction(s): Over-the-Counter (OTC) or Prescription PHARMA
   a. Nutrition Tolerances or Intolerances; Interactions, Efficacy & Absorbency Impacts
7. Medical Provider—Patient-relevant Nutrition Advisor—interactive AI (e.g., Google Gemini)
   a. Conversational AI—Medical Provider Advisory Services w/Patient-aligned Nutrition

Referring now to FIG. 3F there is illustrated the Relevant Nutrition Score Survey for Consumer (user 6 of 12).

### 6. USER 6: RNS OTC/Pharmaceutical and Consumer-aligned Product User App(s) (FIG. 3F).

### A. RNS Assessment Categories for OTC/Pharmaceutical Users: Inputs and Objectives

1. Product/Patient Usage or Intent, Combined with Nutritional Interactions & Efficacies
2. Target Consumer Health Profile, Dietary Impacts, Interactions & Opportunity Uses
3. Medication Impacts upon Dietary Habits, Nutritional Interactions and Target Efficacy
4. Product-User Interactive Impacts of Situational or Conditional Lifestyle/Environment
5. Consumer Preferences, Behaviors or Bio-Impairments Related to Food & Medication
6. Brand Drivers, Product Positioning and Development Insights, Impacts and Efficacies
7. Regulatory and Safety Awareness; Food & Drug Interactions, Side-Effects & Claims

### B. RNS Food, Product or Brand Scores: IRX FOODSCORE—Brands and Products Options

1. PHARMA Brand/Product(s)' General Health Applications, Nutrition Interactions & Insights
   a. Governing Guideline(s) or Prevailing Standard(s): (e.g., US FDA and Bio Efficacies)
2. Target PHARMA Brand(s)/Product(s) Relevance to Objective Health & Interactive Nutrition
   a. Macro Health Objective, Efficacies and Absorption: (e.g., Conditional, Chronic Pain)
3. PHARMA Brand and Product-aligned Integrated Health and Nutrition Impacts and Insights
   a. Micro Health and Patient-specific (e.g., Treatment, Food & Drug Interaction Scores)
4. Purposeful Brand & PHARMA Product Comparative Nutrition Interactions (1, 2 or 3 Above)
   a. Actual or Theoretical Product vs. Product Nutrition Interaction and Efficacy Scores.
   b. PHARMA Brand and Product Nutrition-interaction Risk Assessment & Ranking.

### C. RNS PHARMA Brand/Product-aligned Actionable Insights: User App(s)-Software

1. PHARMA Brand or Product Certification of Nutrition-aligned Interactive Health Efficacy
   a. Relevant Product and Health Condition, Weighted Nutrition Interaction Scoring
2. PHARMA Brand or Product Efficacy (existing or hypothetical) and Nutrient Interactions
   a. Relevant Meal Ingredients and Purposeful Nutrition to Optimize Drug Interactions
3. Efficacy-SCORE Relevance and PHARMA Brand-aligned Product Side-Effect Mitigation
   a. Brand/Product & Target Nutrition-aligned Product Insights: General, Specific Health
4. PHARMA Brand, Product-aligned Environmental Insights and Impacts (existing/proposed)
   a. Brand and Product-aligned Environmental or Food-related Factors (Supply Chain)
5. PHARMA Brand and Product, Packaging, Preservative & Preparational Practices (P5)
   a. Product-relevant P⁵ Factors & Impacts to Target Consumer (Weighted Contributions)
6. Brand-aligned Medication Interaction(s): Over-the-Counter (OTC), Prescription PHARMA
   a. FOOD-MED Tolerances or Intolerances; Interactions, Efficacy & Absorbency Impact
7. PHARMA Brand Relevant Nutrition Advisor—interactive AI (e.g., Google Gemini APPS)
   a. Conversational AI—PHARMA Product Complementary Nutrition Advisory Services.

Referring now to FIG. 3G there is illustrated the Relevant Nutrition Score Survey for Consumer (user 7 of 12).

### 7. USER 7: RNS Educational & Institutional-aligned Users App(s) (FIG. 3G)

### A. RNS Assessment Categories for Educational & Institutional Users: Inputs and Objectives

1. Nutritional Knowledge/Impacts Assessment
2. Objective Dietary Habits and Patterns
3. Research and Evidence-Based Practices
4. Educational and Intervention Strategies
5. Nutritional Assessment Skills & Outputs
6. Interdisciplinary Collaboration & Communication
7. Technology and Innovation in Nutrition

### B. RNS Food, Product/Brand Score: IRX FOODSCORE—Educational/Institutional Options

1. Target Study Nutrition, General Food, Integrated Health Impacts, Ratings, Scores & Insights
   a. Research-specified Guideline(s) or Prevailing Standard(s): (e.g., US FDA; Diabetes)
2. Research-aligned, Purposeful Product & Relevant Nutrition Ratings, Scores, Grades, Insights
   a. Macro Nutrient Health Objectives, Efficacy or Interactions (e.g., Heart Disease)
3. Target Patient Personalized, Integrated Health & Dietary Nutrition Ratings, Scores & Insights
   a. Micro Personal or Consumer Patient-specific (e.g., 50 YO Male w/Crohn's Disease)
4. Research Treatment-related Health/Nutrition (1, 2, 3 Above); Dietary Scores, Grades, Insight
   a. Research-aligned Nutrition-driven Purposeful Lifestyle; Recommended Diet Scores

### C. RNS Education Brand/Product-aligned Actionable Insights: User App(s)-Software

1. Specified Research Lifestyle, Life Stage, Designated Health State-relevant Product(s) Finder
   a. Nutrition-relevant Products, Scores, Sourcing, Ranking for Target/Studied Recipients
2. Evidence-aligned Meal Planning w/Designated Nutrients; Research/Health Outcome-aligned
   a. Study Relevant Meal Ingredients, Intentional Nutrition and Purposeful Recipes
3. Study-specified Store-SCORE Relevant Research: Health-state Product Relevance (SKU's)
   a. Research Condition-relevant Products Locator: General Health or Specific Health
4. Environmental Research Impacts of Study-specified User-Diets (existing or proposed)
   a. Identified Environmental or Specified Identified Food-related Factors (Supply Chain)
5. Research Objective-aligned Product, Packaging, Preservative & Preparational Practices (P5)
   a. Study-relevant P⁵ Impacts of Specific Patient Treatments (Weighted Contributions)
6. Product Study-aligned Medication Interaction(s): Over-the-Counter or Prescription Pharma
   a. Tolerances or Intolerances; Interactions, Efficacy & Absorbency Impacts
7. Institutional—Research-relevant Nutrition Advisor—interactive AI (e.g., Google Gemini)
   a. Conversational AI—Educational Advisory Services with Study-aligned Nutrition

Referring now to FIG. 3H there is illustrated the Relevant Nutrition Score Survey for Consumer (user 8 of 12).

### 8. USER 8: RNS Sports, Fitness & Trainer User Application(s) (FIG. 311)

### A. RNS Assessment Categories for Sports, Fitness & Trainer Users

1. Athletic Profile & Performance Goals
2. Nutritional Knowledge & Purposeful Practices
3. Body Composition & Energy Balance
4. Objective Injury Prevention & Recovery Impacts
5. Sports-Specific Nutritional Needs & Caloric Drivers
6. Psychological Factors in Sports Nutrition
7. Environmental & Social Influences on Athletic Nutrition

### B. RNS Food, Product/Brand Scores: IRX FOODSCORE—Sports/Fitness Scoring Options

1. Athlete or Sport-aligned General Health & Applied Nutrition Ratings, Scores, Grades, Insight
   a. Accepted Guideline(s) or Prevailing Standard(s): (e.g., Low Carb, High Protein Diet)
2. Sports' Relevant Health & Nutrition Ratings, Scores, Grades and Insights
   a. Macro Sport Health Objectives, Injuries, Chronic Ailments: (e.g., Triathletes, Golfer)
3. Athlete-aligned Personal Health & Nutrition Rating, Score, Grade and Insights
   a. Micro Personal or Consumer Target-Specific (e.g., 24 YO Female Rugby Player)
4. Comparative Health & Nutrition (1, 2 or 3 Above); Comparative Scores, Grades and Insights
   a. Sport's Nutrition, Product Side-by Side Attributes; Product vs. Product Scores/Grades

### C. RNS Sports & Fitness/Trainer Actionable Insights: User App(s)/Software Functionality

1. Sport or Athlete-aligned Health-relevant Product(s) Finder
   a. Sport(s), Team or Athlete Relevant Product, Score, Sourcing or Ranking
2. Sport-specific or Athlete intentional Meal Planning, Target Nutrients and Dietary Regimens
   a. Sport, Team or Athlete-Relevant Meal Ingredients, Nutrition and Purposeful Recipes
3. Sport-relevant Store-SCORE Sourcing: Sport-aligned Product Relevance (SKU's)
   a. Sport or Regimen-relevant Product Store Locator: General Health or Specific Health
4. Environmental Impacts of Sport, Team or Athlete User Diets and Foods (existing/proposed)
   a. Known Environmental or Target Food-related Factors; Clean Sourcing, Supply Chain
5. Sport or Athlete-specified Product, Packaging, Preservative & Preparational Practices (P5)
   a. Product-relevant P⁵ Factors & Impacts to Target Consumer (Weighted Contributions)
6. Food-Athlete Medication Interaction(s): Over-the-Counter (OTC) or Prescription PHARMA
   a. Tolerances or Intolerances; Interactions, Efficacy, Absorbency, Performance Impacts
7. Sport/Athlete-relevant Nutrition Advisor: Interactive AI (e.g., Google Gemini App)
   a. Conversational AI—Athlete Nutrition Advisory Services and Suggestive Nutrition

Referring now to FIG. 3I there is illustrated the Relevant Nutrition Score Survey for Consumer (user 9 of 12).

### 9. USER 9: RNS Cooks, Chefs & Culinary Enthusiast User Application(s) (FIG. 3I)

### A. RNS Assessment Categories for Cooks, Chefs & Culinary Enthusiast Users

1. Culinary Background and Expertise
2. Nutritional Knowledge & Purposeful Practices
3. Objective Ingredient Selection and Sourcing
4. Culinary Techniques and Health Considerations
5. Objective Menu Planning, Purpose & Nutritional Balance
6. Target Customer Education & Consumer Communication
7. Innovation, Influences & Trends in Healthy Cuisine

### B. RNS Food, Product/Brand Scores: IRX FOODSCORE—Chef/Culinary Scoring Options

1. Menu, Meal or Foods General Health & Nutrition Rating, Scores, Grades, Insights
   a. Accepted Guideline(s) or Prevailing Standard(s): (e.g., US FDA; Diabetic, Vegan)
2. Meal or Recipe-relevant Health & Nutrition Rating, Scores, Grades and Insights
   a. Macro Health Objectives, Caloric or Ingredient Optimizer (e.g., Seafood Chowder)
3. Target Menu or Cuisine Health & Nutrition Ratings, Scores, Grades and Insights
   a. Micro Menu or Cuisine-Specific (e.g., Greek Gyro with non-Fat Greek Yogurt)
4. Comparative Health & Nutrition (1, 2 or 3 Above); Comparative Scores, Grades and Insights
   a. Product Side-by Side Attributes; Product vs. Product Scores and Grades

### C. RNS Chef/Culinary-aligned Actionable Insights: User App(s) & Software Functionality

1. Menu or Cuisine Certification of Purposeful, Health-relevant Meal Claims & Positioning
   a. Relevant Meal/Recipes Scores, Rankings; Related Health Claims Risk Assessments
2. Cuisine or Productized Meal Planning, Target Ingredients, Nutrients (existing/hypothetical)
   a. Relevant Meal Ingredients, Nutrition and Purposeful Recipes
3. Source-SCORE Relevance: Ingredient-Source-aligned Region or Production Relevance
   a. Menu/Recipe-aligned Relevant Ingredient Source Insights: General or Specific Health
4. Menu or Meal ingredients-aligned Environmental Insights and Impacts (existing/proposed)
   a. Menu, Meal or Recipe-aligned Environmental or Food-related Factors (Supply Chain)
5. Menu or Meal Ingredients Product, Packaging, Preservative & Preparational Practices (P5)
   a. Product-relevant P⁵ Factors & Impacts to Target Consumer (Weighted Contributions)
6. Menu & Meal-aligned Dietary Interaction(s): Seasonal, Regional, Situational or Conditional
   a. Tolerances or Intolerances; Interactions, Efficacy & Absorbency Impacts
7. Cuisine, Meal, Recipe-relevant Nutrition Advisor—interactive AI (e.g., Google Gemini App)
   a. Conversational AI—Chef/Cuisine Nutrition Advisory Services; Suggestive Nutrition

Referring now to FIG. 3J there is illustrated the Relevant Nutrition Score Survey for Consumer (user 10 of 12).

### 10. USER 10: RNS Researchers & Scientist User Application(s) (FIG. 3J)

### A. RNS Assessment Categories for Researchers & Scientist Users

1. Nutrition Research Methodology & Design Frameworks
2. Applicable Data Collection, Analyses & Discovery
3. Target Consumer Participant Characteristics & Recruitment
4. Purposeful Nutritional Intervention or Intentional Exposure
5. Outcome Measures, Metrics & Success Endpoints
6. Translational Potential & Nutritional Application(s)
7. Innovative Personal Approaches & Future Directions

### B. RNS Food, Product/Brand Scores: IRX FOODSCORE—Scientific Scoring Options

1. Scientific Study Nutrition, General Food, Integrated Health Impacts, Scores & Insights
   a. Study-specified Guideline(s) or Prevailing Standard(s): (e.g., US FDA; Obesity)
2. Research-aligned, Purposeful Product & Relevant Nutrition Ratings, Scores, Grades, Insights
   a. Macro Nutrient Health Objectives, Efficacy or Interactions (e.g., Lung Cancers)
3. Studied Population Personalized, Integrated Health & Dietary Nutrition, Scores & Insights
   a. Micro Personal, Studied Patient-specific (e.g., 62 YO Male w/Alzheimer's Disease)
4. Research Treatment-related Health/Nutrition (1, 2, 3 Above); Dietary Scores and Insights
   a. Research-aligned Purposeful Nutrition Lifestyle; Benchmarked Nutrition Scores

### C. RNS Education Brand/Product-aligned Actionable Insights: User App(s)-Software

1. Specified Research Lifestyle, Life Stage, Designated Health State-relevant Product(s) Finder
   a. Nutrition-relevant Products, Scores, Sourcing, Ranking for Target/Studied Recipients
2. Evidence-aligned Meal Planning w/Designated Nutrients; Research/Health Outcome-aligned
   a. Study Relevant Meal Ingredients, Intentional Nutrition and Purposeful Recipes
3. Study-specified Store-SCORE Relevant Research: Health-state Product Relevance (SKU's)
   a. Research Condition-relevant Products Locator: General Health or Specific Health
4. Environmental Research Impacts of Study-specified User-Diets (existing or proposed)
   a. Identified Environmental or Specified Identified Food-related Factors (Supply Chain)
5. Research Objective-aligned Product, Packaging, Preservative & Preparational Practices (P5)
   a. Study-relevant P⁵ Impacts of Specific Patient Treatments (Weighted Contributions)
7. Product Study-aligned Medication Interaction(s): Over-the-Counter or Prescription Pharma
   a. Tolerances or Intolerances; Interactions, Efficacy & Absorbency Impacts
8. Institutional—Research-relevant Nutrition Advisor—interactive AI (e.g., Google Gemini)
   a. Conversational AI—Educational Advisory Services with Study-aligned Nutrition

Referring now to FIG. 3K there is illustrated the Relevant Nutrition Score Survey for Consumer (user 11 of 12).

### 11. USER 11: RNS Active Military & Veteran User Application(s) (FIG. 3K)

### A. RNS Assessment Categories for Active Military & Veteran Users

1. Target User and Objective Soldier Military Service Profile
2. Objective Combat Readiness & Performance Level Nutrition
3. Military-Related Health Conditions “Readiness”
4. Preparation, Transition or Reintegration Challenges
5. Objective Physical Fitness & Target Body Composition
6. Environmental Influencers & Resilience Preparation
7. Long-term Health, Stress Management & Wellness Goals

### B. RNS Food, Product or Brand Scores: IRX FOODSCORE—Military Scoring Options

1. Military-aligned General Health & Applied Nutrition Ratings, Scores, Grades, Insight
   a. Accepted Guideline(s) or Prevailing Standard(s): (e.g., High Protein, Low Fat, etc.)
2. Military Situational-relevant Health & Nutrition Ratings, Scores, Grades and Insights
   a. Macro Military Health Objectives, Injuries, Situations (e.g., Desert, Mountain, etc.)
3. Military-aligned Personal Health & Nutrition Product Ratings, Scores, Grades and Insights
   a. Micro Military Personnel Target-Specific (e.g., 28 YO Female Army Ranger)
4. Comparative Health & Nutrition (1, 2 or 3 Above); Comparative Scores, Grades and Insights
   a. Military Nutrition, Product Side-by Side Attributes; Product vs. Product Scores

### C. RNS Military & Veteran Actionable Insights: User App(s)/Software Functionality

1. Military & Veteran-aligned Health-relevant and Objective Product(s) Finder
   a. Military (Active or Inactive Duty) Relevant Products, Scores, Sourcing or Ranking
2. Military & Veteran intentional Meal Planning, Target Nutrients and Dietary Regimens
   a. Military (Active/Inactive) Relevant Meal Ingredients, Nutrition, Purposeful Recipes
3. Military or Veteran-relevant Store-SCORE Sourcing: Military-aligned Product Relevance
   a. Military (Active/Inactive) Relevant Product Store Locator: General or Specific Health
4. Military & Veteran Environmental Impacts of Service or Situational User Diets and Foods
   a. Military (Active/Inactive) Environmental, Situational Food-related Factors; Sourcing
5. Military & Veteran-specified Product, Packaging, Preservative & Preparational Practices (P5)
   a. Product-relevant P⁵ Factors & Impacts to Target Consumer (Weighted Contributions)
6. Military Personnel Food—Medication Interaction(s): OTC or Prescription PHARMA
   a. Tolerances or Intolerances; Interactions, Efficacy, Absorbency, Performance Impacts
7. Military Personnel-relevant Nutrition Advisor: Interactive AI (e.g., Google Gemini App)
   a. Conversational AI—Military Nutrition Advisory Services and Suggestive Nutrition

Referring now to FIG. 3L there is illustrated the Relevant Nutrition Score Survey for Consumer (user 12 of 12).

### 12. USER 12: RNS Governmental, USDA/FDA & Agency Application(s) (FIG. 3L)

### A. RNS Assessment Categories for Governmental, USDA/FDA & Agencies

1. Target Population/Consumers Health Assessment Metrics
2. Target Regulatory Frameworks & Policy(s) Objectives
3. Target Food System, Marketplace & Supply Chain Analyses
4. Target Consumer(s) Behavior & Education Objectives
5. Public Health Concerns & Intentional Nutrition Interventions
6. Food Industry Collaboration & Oversight Objectives
7. Objective Research & Nutritional Science(s) Innovations

### B. RNS Food, Product or Brand Scores: IRX FOODSCORE—Agency Scoring Options

1. Guideline Frameworks General Health & Nutrition Exchange Platform and Scores Insights
   a. Accepted Guideline(s) or Prevailing Standard(s): (e.g., US FDA; Food Agency, etc.)
2. Agency Guideline or Objective-relevant Health & Nutrition Ratings, Scores and Insights
   a. Agency Macro Health Objectives, Afflictions or Diseases: (e.g., Cancer or Diabetes)
3. Agency defined Target Population Health & Nutrition Ratings, Scores, Grades and Insights
   a. Agency Macro, Micro or Consumer Target-Specific (e.g., Teenage Girls with Cancer)
4. Comparative Health & Nutrition (1, 2 or 3 Above); Comparative Scores, Grades and Insights
   a. Target Product Side-by Side Attributes; Product vs. Product Compliance Scores

### C. RNS Governmental, Agency-aligned Actionable Insights: User App(s) & Software

1. Specified Guidelines: General Compliance or Conditional Health State-relevant Food Claims
   a. Nutrition-specific Compliance, Food Policy, Ingredient Scores per Agency Guidelines
2. Agency Guideline and Evidence-aligned Meal Planning against specified Health Outcomes
   a. Specifying Relevant Meal Ingredients, Intentional Nutrition and Purposeful Claims
3. Agency-specified Compliance-SCORE Relevant Research: Health-state Product Relevance
   a. Agency-aligned Target Health and Nutrition-relevant Products Compliance Benchmarks and Standards: Agency-specified General Health or Specific Health
4. Environmental Impacts of Agency-specified Guidelines of User-Diets (existing or proposed)
   a. Identified Environmental or Specified Identified Food-related Factors (Supply Chain)
5. Agency Objective-aligned Products, Packaging, Preservative & Preparational Practices (P5)
   a. Agency-relevant P⁵ Impacts of Specific Patient Treatments (Weighted Contributions)
6. Agency Product-aligned Medication Interaction(s): Over-the-Counter or Prescription Pharma
   a. Tolerances/Intolerances; Interactions, Efficacy & Absorbency Impacts (Transparency)
9. Agency Advisor—Guideline-relevant Nutrition Advisor: Interactive AI (e.g., Google Gemini)
   a. Conversational AI: Agency Compliance Advisory Service: Health-aligned Nutrition

Identifying the user group is beneficial for RNS AI because it immediately narrows the goals and priorities set by RNS when assessing nutrition. This allows RNS AI to provide more accurate scores and feedback to the user before answering any questionnaire questions.

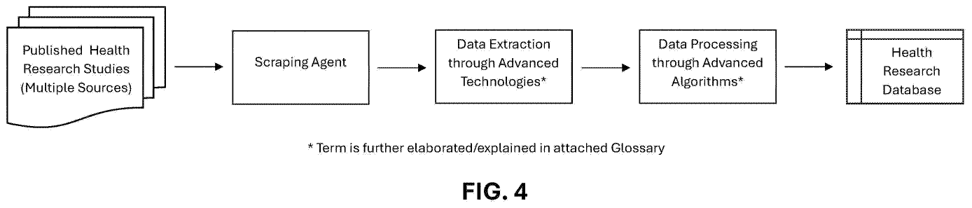

Referring now to FIG. 4, once the User Profile for Calculation is prepared as shown above in FIG. 2, the user's survey answers are cross-referenced by RNS AI with an internally stored Health Research Database compiled by IRX. This database is dynamically compiled from various public and private sources including PubMed Research through methods further described under Advanced Technologies (2) as defined in the Glossary. This amalgamation of disparate datasets is processed and combined through Advanced Algorithms as defined in the Glossary (see Term 3 in Glossary) to create a singular IRX Reference Data Lake, or Internal Health Research Database. This process is summarized in FIG. 4.

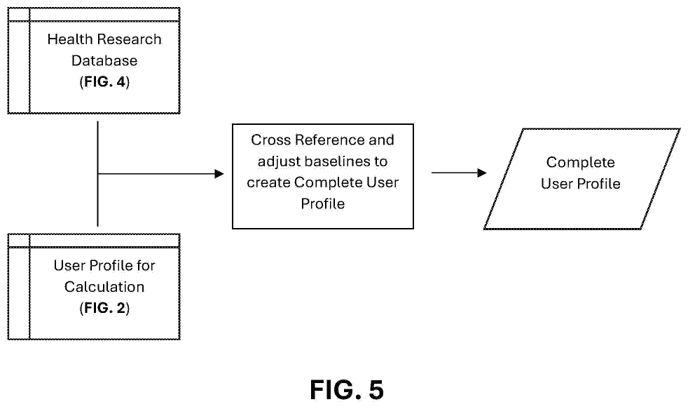

Referring now to FIG. 5, the Health Research Database is referenced by RNS AI to create weighted values for a dynamic list of health variables that is subject to change over time given available research. Currently, RNS AI assesses 167+ variables of food products including Basic Components, Vitamins, Minerals, Saturated Fats, Monounsaturated Fats, Polyunsaturated Fats, Amino Acids, Other Nutrition, Nutrient Exchanges, and Other Contributing Health Impact Factors (see Table 1 below). The system then creates a baseline for each variable given the average/standard individual or group and then adjusts each baseline as needed given the user's responses to the survey questions and available health research. This completes the user Profile, and it is ready to be used by RNS AI to score/grade food items.

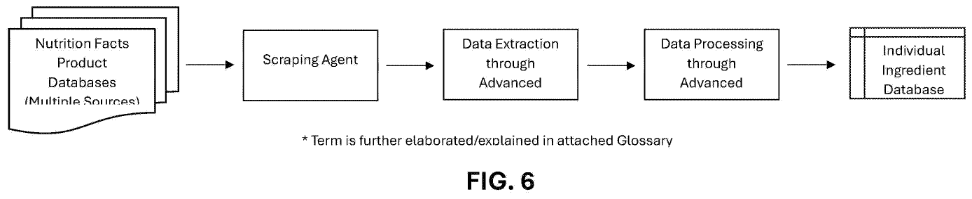

Referring now to FIG. 6, in order to initiate food profile preparation by the system, IRX RNS AI curates an in-house Food Ingredient Database that is dynamically compiled from various public and private sources through methods further described under Advanced Technologies (2) as defined in the Glossary. Each ingredient is stored with relevant, available and specific information including but not limited to source location, growing processes, processing techniques, storage methods, harvest date, packaging, etc. For example, freeze dried unsweetened Organic blueberries from Supplier A in Oregon, which were harvested in July 2023 are saved separately from oven dried blueberries from Supplier B in California that were harvested in September 2023. These differences are important to note because there are numerous variables that influence the nutrition inside a food item including location grown/raised, pesticides/herbicides used, seed source, feed source, harvesting techniques, and processing methods. All the ingredient datasets are then processed and combined through Advanced Algorithms (Term 3 in Glossary) to create a singular IRX Ingredient Reference Data Lake, or Internal Food Ingredient Database. This process is summarized in FIG. 6.

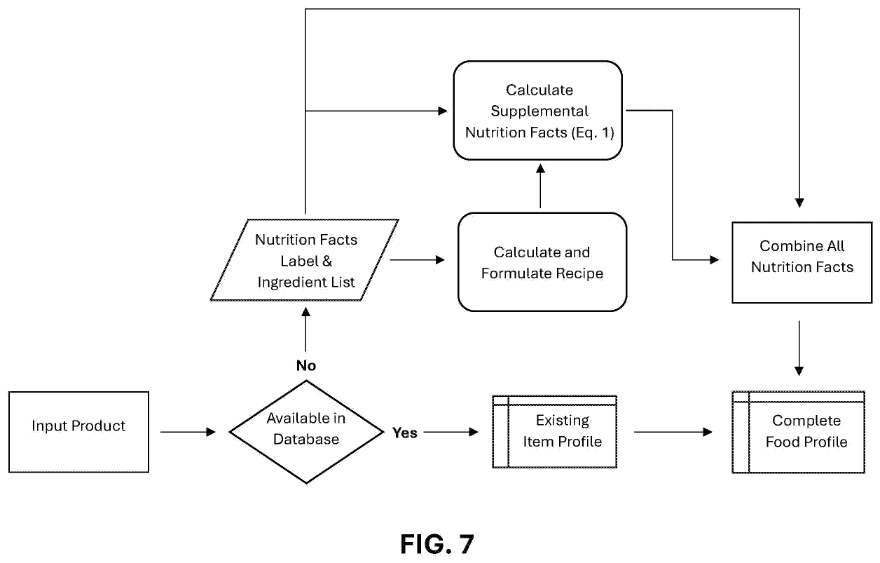

Referring now to FIG. 7, when the user wants to score/grade a product, they can simply scan the UPC Code on the product, search for the product in our database, or add an ingredient/product that is missing. For products existing in our database, the stored information is referenced by RNS AI as the Complete Food Profile for grading. For products not existing in the database, RNS AI takes in variables from the food product starting with the Nutrition Facts Label and ingredient list and cross-references them to calculate the most likely recipe of the given food item if the recipe is not available/submitted. Supplemental nutrition facts (Nutrients not listed on the Nutrition Facts Label) are generated using this recipe and average density values of nutrients per ingredient referenced from our Food Ingredient Database. A non-limiting example is if 10% of a 100 g recipe is raw blueberries, and the average Vitamin C in 100 g of raw blueberries is 9.7 mg, we can calculate the Vitamin C added to the recipe from the blueberries using Reference Equation 1 (below):

$$( 10 \% \times 100 g ) \times ( 9.7 mg / 100 g ) = 0.97 mg of Vitamin C \qquad Reference Equation 1$$

$$( Recipe \% \times Serving Size ) \times ( Nutrient Amount per 100 g / 100 g ) = Nutrient per Serving Size$$

This process is repeated for each nutrient in each ingredient in the ingredient statement that does not appear in the Nutrition Facts Label. These values are utilized to create a Food Profile for the given food product that contains values and sources for each of the listed nutrients in the Complete Food Profile List (below). Each nutrient is listed in the Appendix and further described/defined in the Glossary. Once the Supplemental Nutrition Facts are completed, they are appended to the base Nutrition Facts to create the Complete Food Profile. This process is illustrated in FIG. 7.

Next, Relevant Nutrition Score (RNS) is calculated. With the Completed User Profile and Food Profile, RNS AI has everything it needs to calculate the Relevant Nutrition Score of the food item for the user. Referencing the User Profile's target amounts and ranges for each food variable, RNS AI compares the information in the Complete Food Profile to assign a coefficient value for each food variable. These values are combined via IRX Proprietary Formulations to create an overall Health Grade/Score that ranges 0-100 for the item being graded. These formulations are represented by Equations 2-3B below.

$$RNS = {\sum }_{i = 1}^{9} ( {δ}_{i} x {G}_{i} ) \qquad Equation 2$$

Where in Equation 2 Gi can be substituted for any of:
G₁=Basic Components (40)
G₂=Vitamins (29)
G₃=Minerals (17)
G₄=Saturated Fats (13)
G₅=Monounsaturated Fats (8)
G₆=Polyunsaturated Fats (10)
G₇=Amino Acids (18)
G₈=Other Components (24)
G₉=Exchanges (8)

TABLE 1
BASIC COMPONENTS

| | |
|---|---|
| 1. | Gram Weight |
| 2. | Calories |
| 3. | Calories from Fat |
| 4. | Calories from Saturated Fat |
| 5. | Calories from Trans Fat |
| 6. | Protein |
| 7. | Carbohydrates |
| 8. | Total Dietary Fiber |
| 9. | Total Soluble Fiber |
| 10. | Total Insoluble Fiber |
| 11. | Dietary Fiber |
| 12. | Soluble Fiber |
| 13. | Insoluble Fiber |
| 14. | Other Dietary Fiber |
| 15. | Other Soluble Fiber |
| 16. | Other Insoluble Fiber |
| 17. | Total Sugars |
| 18. | Added Sugars |
| 19. | Monosaccharides |
| 20. | Fructose |
| 21. | Galactose |
| 22. | Glucose |
| 23. | Disaccharides |
| 24. | Lactose |
| 25. | Maltose |
| 26. | Sucrose |
| 27. | Other Carbs |
| 28. | Net Carbs |
| 29. | Starch |
| 30. | Carbohydrates (available) |
| 31. | Fat-Total |
| 32. | Saturated Fat |
| 33. | Monounsaturated fat |
| 34. | Polyunsaturated fat |
| 35. | Trans Fatty Acids |
| 36. | Salatrims |
| 37. | Cholesterol |
| 38. | Water |
| 39. | Ash |
| 40. | Kilojoules |

TABLE 2
VITAMINS

| | |
|---|---|
| 41. | Vitamin A (IU) |
| 42. | Vitamin A (RE) |
| 43. | Vitamin A (RAE) |
| 44. | Retinol RE |
| 45. | Carotenoid RE |
| 46. | Alpha Carotene |
| 47. | Beta Carotene |
| 48. | Beta Carotene Equiv. |
| 49. | Vitamin B1 Thiamin |
| 50. | Vitamin B2 Riboflavin |
| 51. | Vitamin B3 Niacin |
| 52. | Niacin Equivalents |
| 53. | Vitamin B6 |
| 54. | Vitamin B12 |
| 55. | Biotin |
| 56. | Folate (Total) |
| 57. | Folic Acid |
| 58. | Folate (DFE) |
| 59. | Folate (Food) |
| 60. | Pantothenic Acid |
| 61. | Vitamin C |
| 62. | Vitamin D (IU) |
| 63. | Vitamin D (mcg) |
| 64. | Vitamin E (IU) |
| 65. | Vitamin E (α-tocopherol) |
| 66. | Vitamin E (α-tocopherol equivalent) |
| 67. | Vitamin E (mg) |
| 68. | Cryptoxanthin |
| 69. | Vitamin K |

TABLE 3
MINERALS

| | |
|---|---|
| 70. | Boron |
| 71. | Calcium |
| 72. | Chloride |
| 73. | Chromium |
| 74. | Copper |
| 75. | Fluoride |
| 76. | Iodine |
| 77. | Iron |
| 78. | Magnesium |
| 79. | Manganese |
| 80. | Molybdenum |
| 81. | Phosphorus |
| 82. | Potassium |
| 83. | Selenium |
| 84. | Sodium |
| 85. | Salt |
| 86. | Zinc |

TABLE 4
SATURATED FATS

87. 4:0-Butyric
88. 6:0-Caprioc
89. 8:0-Caprylic
90. 10:0-Capric
91. 12:0-Lauric
92. 14:0-Myristic
93. 15:0-Pentadecanoic
94. 16:0-Palmitic
95. 17:0-Heptadecanoic
96. 18:0-Stearic
97. 20:0-Arachidic
98. 22:0-Behenic
99. 24:0-Tetracosanoic

TABLE 5
MONOUNSATURATED FATS

100. 14:1-Myristoleic
101. 15:1-Pentadecenoic
102. 16:1-Palmitoleic
103. 17:1-Heptadecenoic
104. 18:1-Oleic
105. 20:1-Gadoleic
106. 22:1-Erucic
107. 24:1-Nervonic

TABLE 6
POLYUNSATURATED FATS

108. 18:2-Linoleic
109. 18:3-Linolenic
110. 18:4-Stearidonic
111. 20:3-Eicosatrienoic
112. 20:4-Arachidonic
113. 20:5-EPA
114. 22:5-DPA
115. 22:6-DHA
116. Omega 3 Fatty Acids
117. Omega 6 Fatty Acids

TABLE 7
AMINO ACIDS

118. Alanine
119. Arginine
120. Aspartic Acid
121. Cystine
122. Glutamic Acid
123. Glycine
124. Histidine
125. Isoleucine
126. Leucine
127. Lysine
128. Methionine
129. Phenylalanine
130. Proline
131. Serine
132. Threonine
133. Tryptophan
134. Tyrosine
135. Valine

TABLE 8
OTHER

136. Alcohol
137. Caffeine
138. Artificial Sweeteners
139. Artificial Preservatives
140. Artificial Colors
141. Colorants
142. Aspartame
143. Saccharin
144. Sugar Alcohols-Total
145. Erythritol
146. Glycerol
147. Inositol
148. Mannitol
149. Sorbitol
150. Xylitol
151. Organic Acids-Total
152. Acetic Acid
153. Citric Acid
154. Lactic Acid
155. Malic Acid
156. Choline
157. Taurine
158. Lutein & Zeaxanthin
159. Lycopene
160. Cost
161. Glycemic Index
162. Glycemic Load
163. Flavor Enhancers
164. Pesticides
165. Herbicides
166. Insecticides
167. Fungicides
168. Heavy Metals
169. Dioxins
170. PCBs
171. Other Environmental
Contaminants
172. Packaging
173. BPA-Packaging
174. HDPE-Packaging
175. LDPE-Packaging
176. PP-Packaging
177. PS-Packaging
178. PVC-Packaging
179. PLA-Packaging
180. PHA-Packaging
181. Other Bioplastic-
Packaging
182. Paperboard-Packaging
183. Corrugated Cardboard-
Packaging
184. Paper-Packaging
185. Molded Fiber-
Packaging
186. Aluminum-Packaging
187. Steel/Tin-Packaging
188. Metal-Packaging
189. Glass-Packaging
190. Plastic Films &
Laminates
191. EPS-Packaging
192. PFAS-Packaging
193. Phthalates
194. Other Packaging
Chemicals
195. Natural Toxins
196. Acrylamide
197. Other Processing
Contaminants

TABLE 9
EXCHANGES

198. Ex-Starch
199. Ex-Other Carb
200. Ex-Vegetable
201. Ex-Fruit
202. Ex-Nonfat Milk
203. Ex-Lean Meat
204. Ex-Alcohol
205. Ex-Fat

While Equation 2 represents the most summarized mathematical explanation of this calculation, Equations 3A-3B expand each nutrient group into their individual nutrients.

$$RNS = {\sum }_{i = 1}^{N} ( {δ}_{i} x {N}_{i} ) \qquad Equation 3 A$$

$$RNS = {δ}_{1} ( GrantWeight ) + {δ}_{2} ( Calories ) + {δ}_{3} ( CaloriesFromFat ) + … + {δ}_{167} ( ExFat ) \qquad Equation 3 B$$

$$EQUATION TERM DEFINITION : Where {δ}_{i} ( delta - i ) represents the nutrient coefficient calculated by RNS AI using the Complete User profile and Food Profile .$$

This Health Grade represents the first half of the Relevant Nutrition Score and for the second half, RNS AI assesses and calculates Food Density Factors for several key nutrient groups. These groups are including but not limited to: Fats, Sugars, Proteins, and Sodium. RNS assesses key ratios for each group and creates a density factor for each. This is a key step in the process because the balance between the subparts of each group is just as important, if not more than the sum. For example, not all protein sources are created equal as some are identified as complete proteins versus incomplete proteins. Complete proteins contain all nine essential amino acids which the human body cannot produce on its own. These amino acids play vital roles in muscle growth, tissue repair, immune function, hormone production, and neurotransmitter synthesis. If any of these amino acids are missing, these various processes can become less efficient or inoperable. This relationship highlights the importance of having not only a variety of these various subcomponents, but also specific relationships between them. All of this is considered by RNS AI when calculating a Density Factor for each group and then combining all group factors into one Food Density Factor.

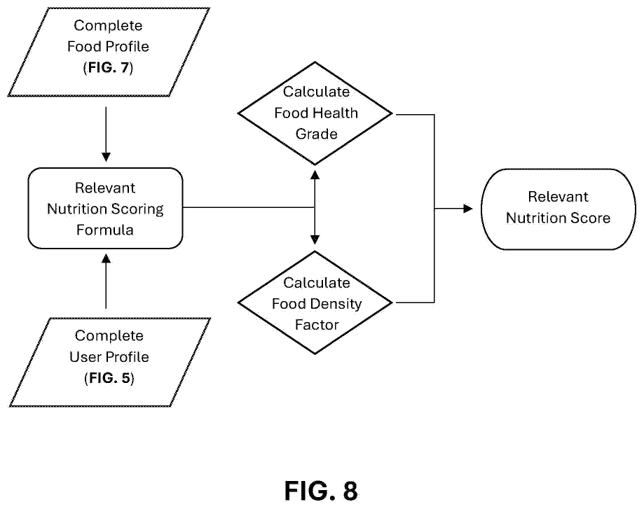

Referring now to FIG. 8, there is illustrated the calculation of Relevant Nutrition Score (RNS). RNS AI combines the Food Health Grade and the Food Density Factor to create the final Relevant Nutrition Score which is a score 0-100 that is accompanied by Letter Grade (A-E) that represents how well the scored food item aligns with the user's profile. This final process is illustrated in FIG. 8.

One anticipated function of the present Systems and Methods for Artificial Intelligence Analyses and Scoring of Consumables is to use RNS AI values to optimize and create new foods. Thus, by using the system described above, RNS AI can go way beyond just scoring foods and can function to optimize existing foods and food formulations, and help create new foods as well as new food formulations.

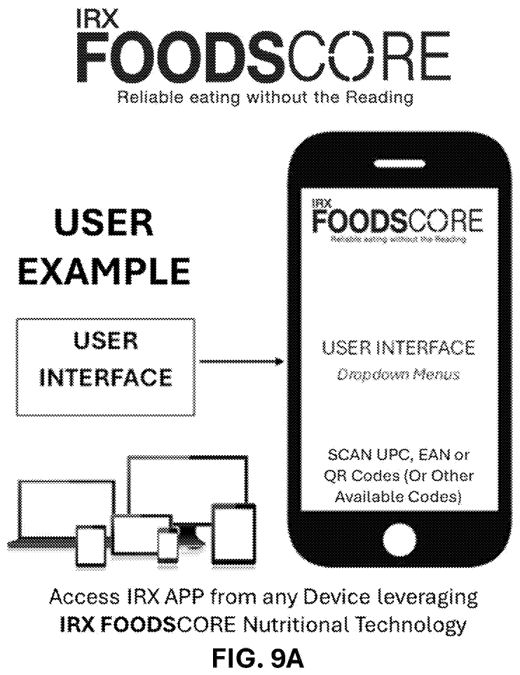

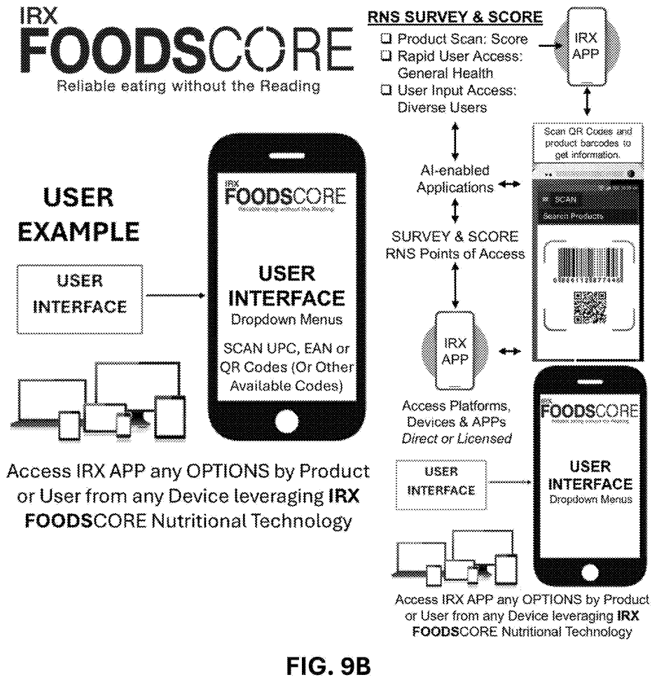

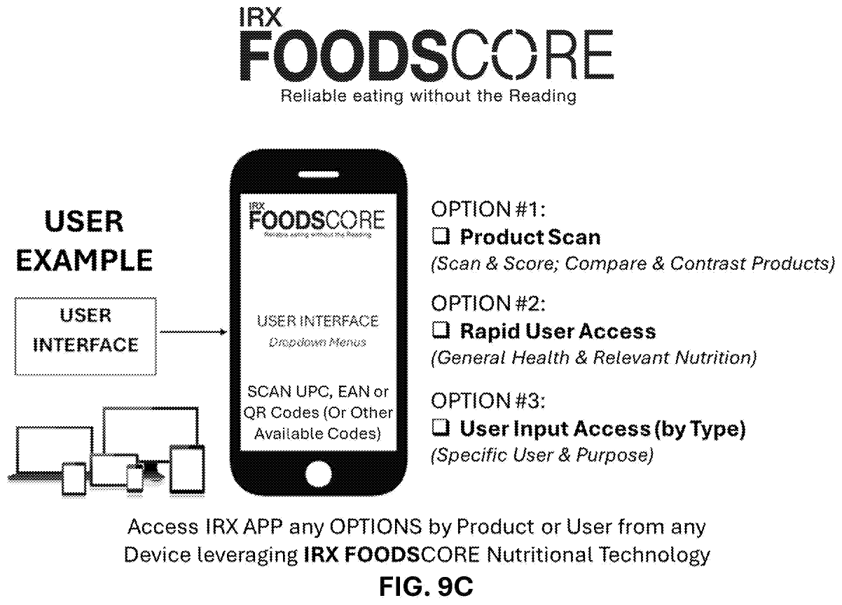

Furthermore, the present Systems and Methods for Artificial Intelligence Analyses and Scoring of Consumables and using RNS AI is designed to be accessible and user friendly on all platforms. For a user to access/use RNS they will use an app (application) on any device able to leverage the technology and access the internet. This app is the IRX FOODSCORE App and the most popular option for access will most likely be a smartphone or other smart device. This user interface is illustrated on a smartphone/mobile device in FIG. 9A.

When the user first opens the app, they will be greeted with three options: Product Scan, Rapid User Access, and User Input Access. These three options are shown in FIG. 9B-9C. If the user selects the first option, it allows them to immediately scan a product code and score it using the general health baseline in the RNS database. To explain, the user declines the option to provide any information in order to quickly score any product and the score will be generated referencing the general health baseline at the core of RNS. It will not be adjusted for any variables because the system does not possess any information about the user.

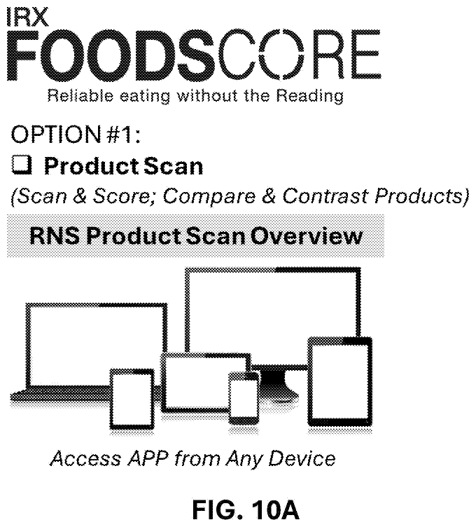

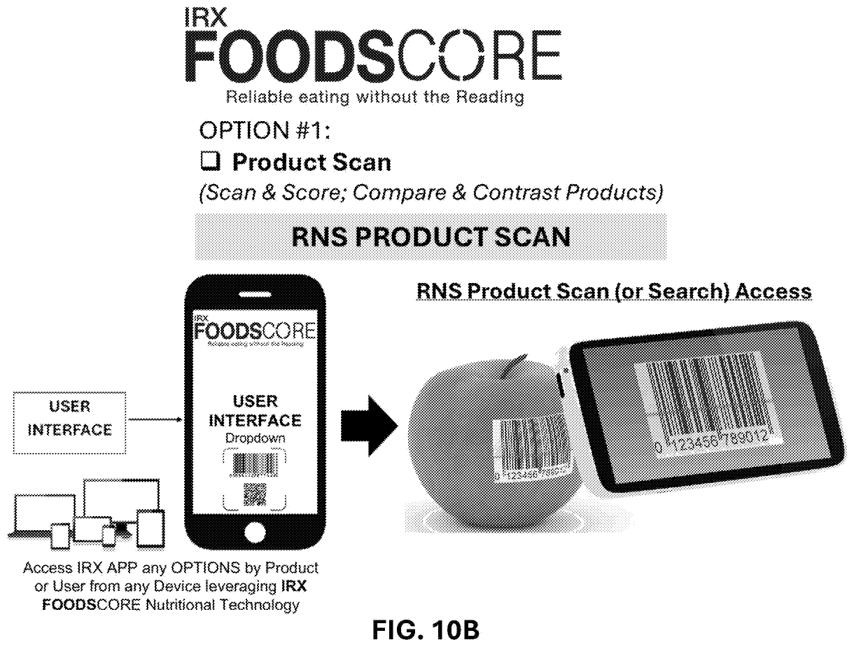

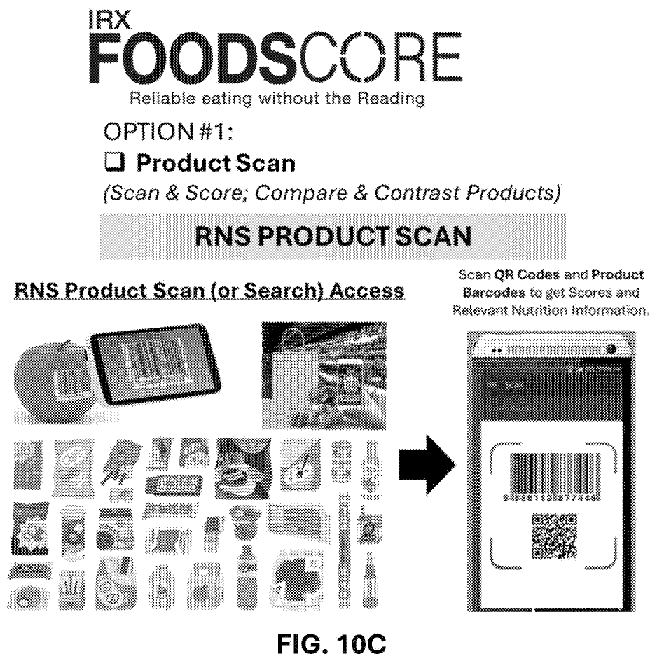

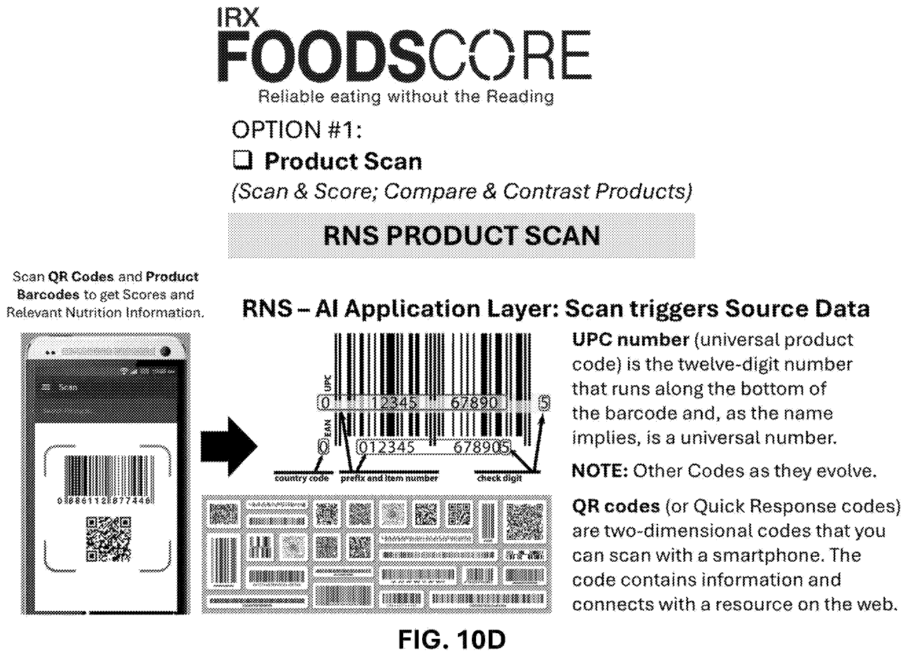

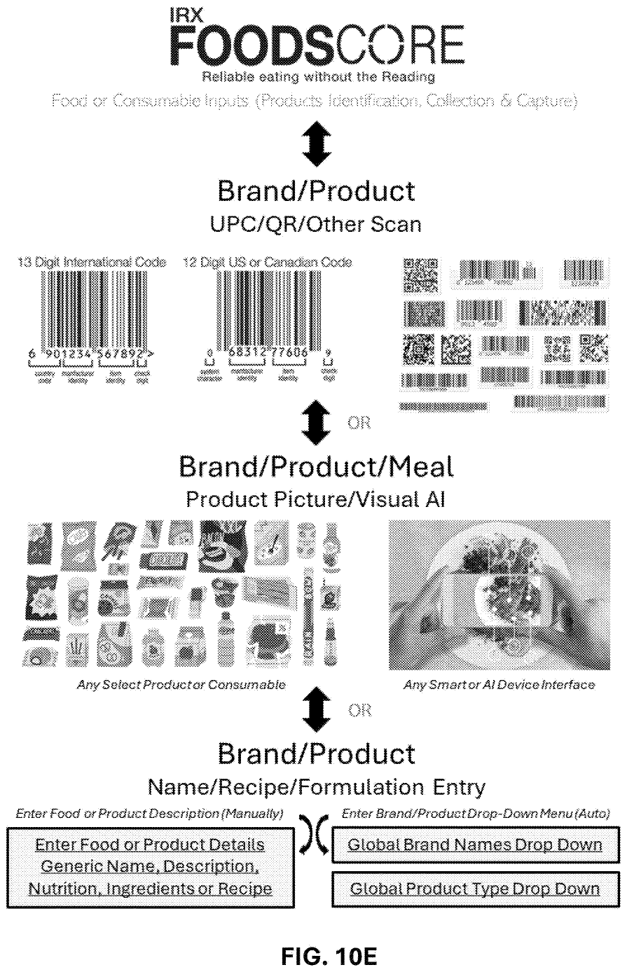

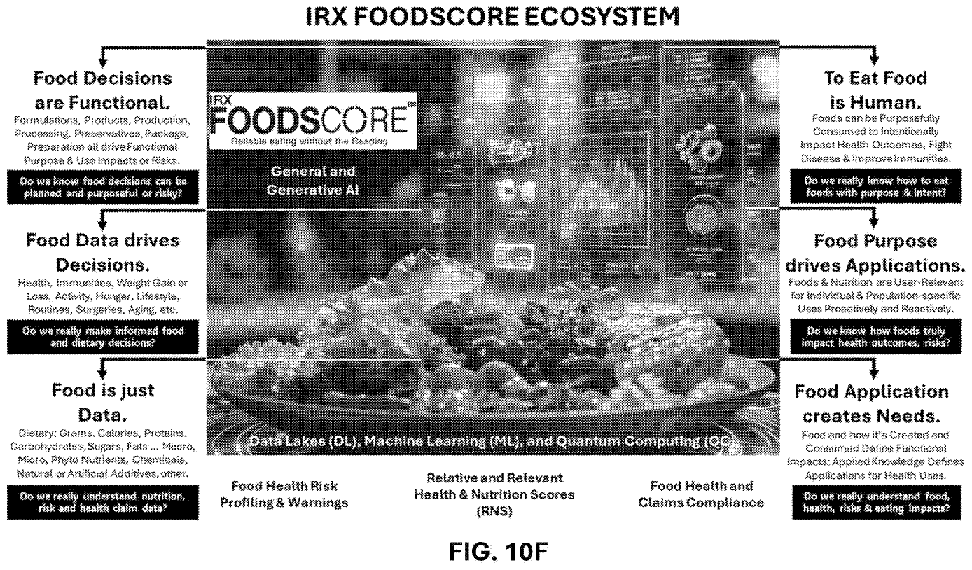

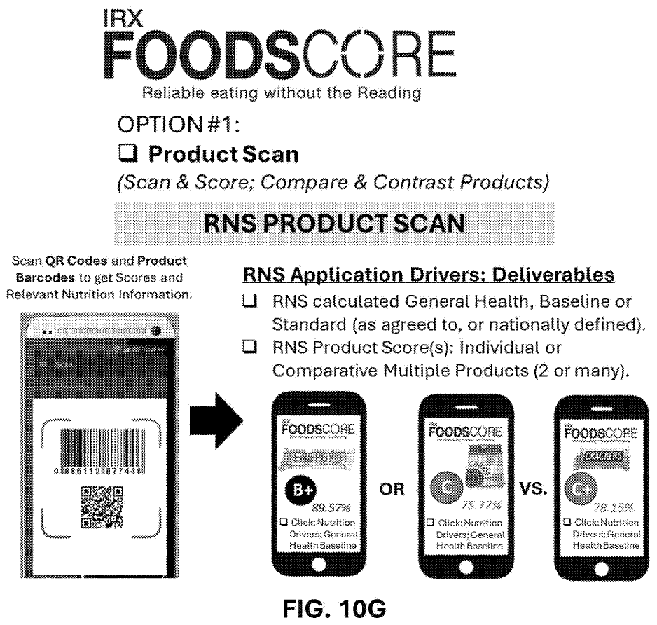

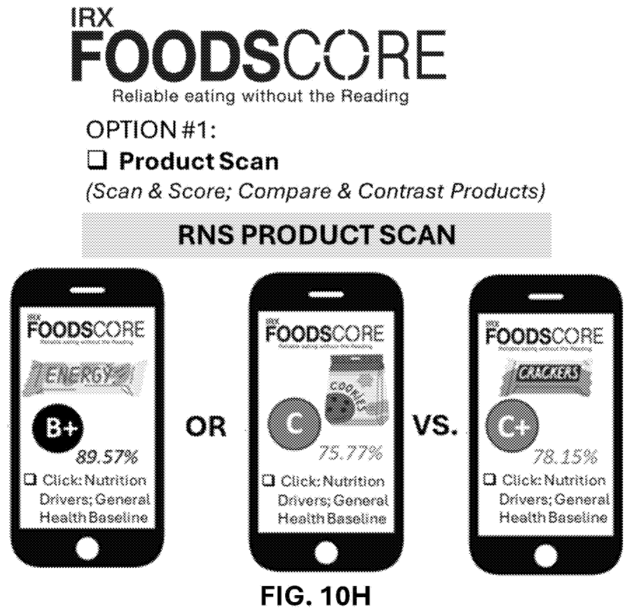

When scanning a product, the user can use their device's camera to scan any identifying codes on the product (or enter it manually), e.g., UPC codes, QR Codes, or other codes available or not yet created. This process of selecting option 1 and scanning a product are illustrated in FIG. 10A-D. Alternatively, if there are not any identifying codes on the product, the user can search the IRX product database and select the matching product or manually add details if not available. Additionally, the user will have the option to use a camera interface device to identify the product through visual AI and any other camera and image recognition technology available.

These options are depicted in FIG. 10E. Another benefit of capturing information with visual AI is the ability to capture and store additional information related to the product, meal, or consumable such as: color, texture, shape and size, symmetry and arrangement, individual ingredients, cuisine type, processing level, preparation methods, eating environment, plate characteristics, temporal data, volume, weight, known allergen, and other comparative metrics.

After the product or consumable is identified and captured, RNS AI system collects and digitizes all available and relevant health impact information as summarized in FIG. 10F, and then scores product or consumable as previously described. This score is displayed to the user on their device as depicted in FIG. 10G-H. The figure further shows the app's ability to grade an additional product and display two or more scores at once for comparison. This feature allows user to compare foods to see which is scored higher and why, based upon user defined relevance.

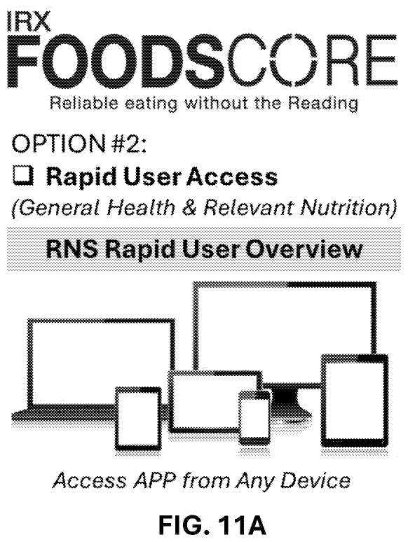

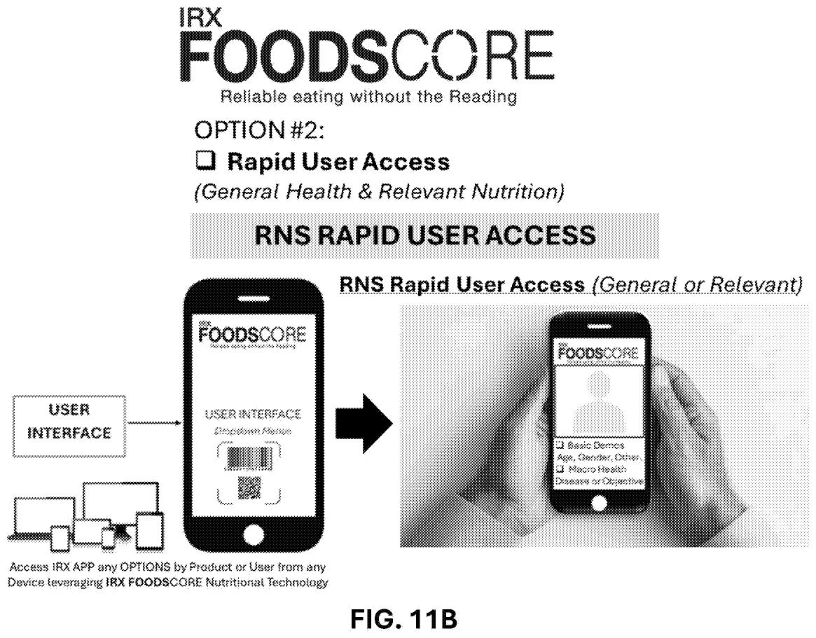

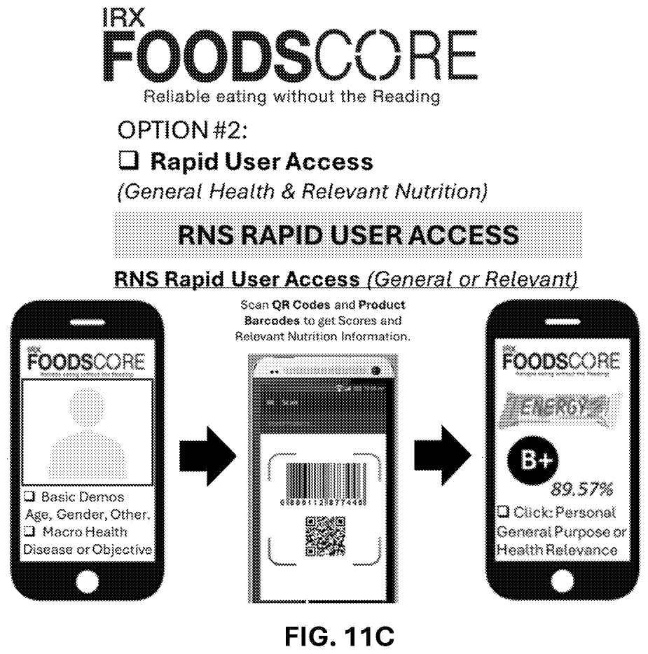

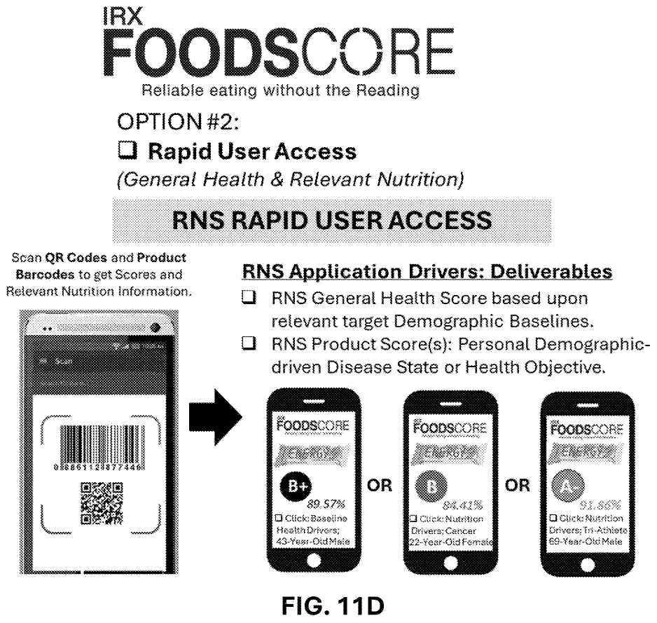

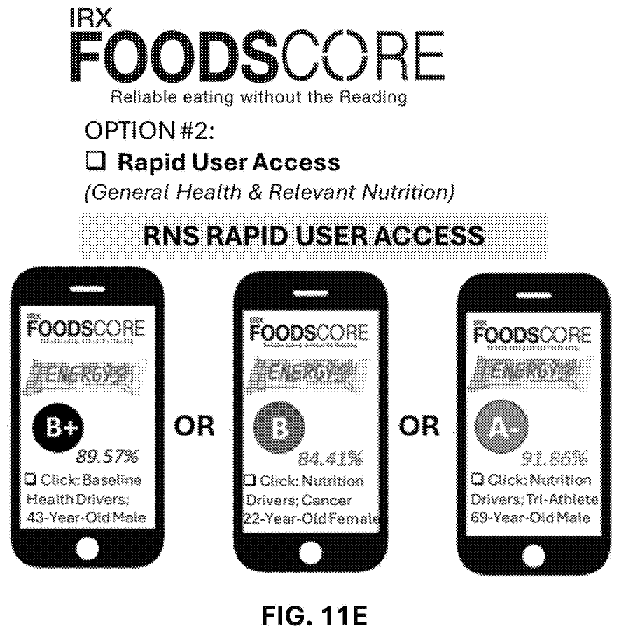

Alternative to Option 1 where a user can immediately score products for general health, they can select Option 2 for Rapid User Access. This option provides the user with a quick way to score a product and receive a more relevant score to them. This is achieved by presenting the user with a brief survey asking a few key questions to establish important baselines. These questions initially include base demographics such as age, sex, ethnicity, home address, weight, activity level, and disease state. This is illustrated in FIG. 11A-B. Given this limited information, RNS can provide the user with a more relevant nutrition score because it is able to more accurately estimate the user's health needs. To explain, a 43-year-old male office worker, a 22-year-old female with cancer, and a 69-year-old male triathlete all require significantly different nutrition to optimize their health. This is illustrated in FIG. 11C-11E.

The final option available to the user is User Input Access (by Type) illustrated in FIG. 12A. In this option, they are presented with the 12 initial user groups and are required to select a group to start in (FIG. 12B shows an example for Consumer). Upon selecting a group, user will be presented with question groups to open and review demographic questions (FIG. 12C-E). As previously discussed, the user can answer as many or as few questions as they want, but more answers provided will allow system to provide more personalized Relevant Nutrition Score.

Referring now to FIG. 12A through FIG. 12E, there is illustrated the various elements for RNS input to access food relevance, here depicting a Consumer User Example. Initially, IRX FOODSCORE utilizes seven key elements (+/− elements in the future) from an individual consumer's unique perspective. Individual consumers can assess existing or prospective food(s), drug interactions and optimized efficacies, or other consumables, and their respective impacts in support of an informed, objective or imposed life stage or lifestyle, or for calculating an average, purposeful or conditional Relevant Nutrition Score (RNS):

### Ranges and Weights of RNS Algorithmic Influence*:

*Utilizing all available, ever-evolving ArtificialIntelligence (AI), Artificial General Intelligence (AGI) and Artificial Superintelligence (ASI): e.g., Utilizing Data-driven Generative AI

### RNS User Example (1 of 12 Current Users)

### Individual Consumer

NOTE: User ability to OPT IN/OUT of any Questions; completing any or all questions desired or option to download relevant records (e.g., Medical History, Military Service, Athletics, etc.) Consumer Filters: Seven (7) Initial Elements (weights and influences are dynamic over time)

| | Dynamically |
|---|---|
| | Weighted Influences: |
| 1.) Personal & Demographics | |
| a) RNS Starting Weight of Influence: | 13% |
| b) RNS Algorithm Dynamic Range: | 8%-17% |
| 2.) Personal Medical History | |
| a) RNS Starting Weight of Influence: | 21% |
| b) RNS Algorithm Dynamic Range: | 14%-25% |
| 3.) Family Health History | |
| a) RNS Starting Weight of Influence: | 14% |
| b) RNS Algorithm Dynamic Range: | 10%-20% |
| 4.) Lifestyle & Behavioral Factors | |
| a) RNS Starting Weight of Influence: | 14% |
| b) RNS Algorithm Dynamic Range: | 7%-17% |
| 5.) Mental Health & Wellbeing | |
| a) RNS Starting Weight of Influence: | 11% |
| b) RNS Algorithm Dynamic Range: | 7%-17% |
| 6.) Environmental & Social Influences | |
| a) RNS Starting Weight of Influence: | 12% |
| b) RNS Algorithm Dynamic Range: | 6%-18% |
| 7.) Health Goals & Concerns | |
| a) RNS Starting Weight of Influence: | 15% |
| b) RNS Algorithm Dynamic Range: | 5%-18% |
| TOTAL of Weighted Initial Element Influence: | 100% |

NOTE:
Key elements continuously evolve (increase or decrease); weighted influence is dynamic.

Each element contributes uniquely to an individual consumer user's nutritional needs, food preferences and (in)tolerances.

Sample clarification of applications of inputs:
1) Age and gender influence metabolic rates, nutrient requirements and health drivers.
2) Personal and family medical histories may indicate predispositions to certain health conditions that dietary and proactive nutritional choices may mitigate or exacerbate.
3) Lifestyle factors: e.g., physical activity or daily routine affect energy needs, meal timing, etc.
4) Mental health/wellbeing are linked to diet; certain foods impact mood and cognitive function.
5) Environmental and social factors, such as cultural and regional food practices and access to fresh produce, shape dietary habits and availability.
6) Individual health goals/concerns impact nutrition requirements, caloric intake, weight targets and management, muscle gain, disease prevention; guiding specific applied dietary strategies.

In summary and by integrating diverse user (e.g., individual consumer) input elements, RNS dynamically scores selected, targeted or available food products. User-aligned and specific food recommendations are individually tailored to holistic health and preventative well-being, while also identifying the relevant functional, reactive or required nutrition intake necessary to achieve health objectives or to assist in a fight against specific diseases, disorders or chronic health states, while addressing immediate nutritional needs, opportunities and desired long-term health outcomes and objectives.

Importantly, RNS beginning weights provide initial and directional baselines for assessing unique influences of each element upon relevant, functional & purposeful food scores:
1) RNS AI applications and algorithms consider unique and dynamic weight ranges, ever-adapting, changing & evolving over time.
2) RNS elements and factorials uniquely consider each one's weighted relevancy for a target consumer/group, attributing weights to individual influential factors and impacts to health.
3) RNS factors adapt and change dynamically based upon inputs and direct feedback, allowing for ongoing, personalized and purposeful dietary adjustments or updated product relevancy scores, consistent with health.
4) RNS elements and factors are persistently redefined, updated and re-prioritized persistently based upon research, medical data and nutritional studies, all considering target populations, unique cultures and impacts.

OPTIONAL OPT-IN USER SURVEY(s): INDIVIDUAL CONSUMER EXAMPLE Range and Weights of Algorithmic Influence: Dynamically adapted as/if answers completed. (Consumer example for illustration Only; Actual Questions, responses and weights may differ)

### 1.A. Personal/Demographic Inputs: Initial 10 Dynamic Questions/Weighted Influence (More Questions Answered, More Relevant RNS FOODSCORE, HIPPA, Regulatory Compliant)

| QUESTIONS: (1-10) | ANSWERS; options, opt-ins, opt-outs, custom, etc. |
|---|---|
| 1) What is your current age? | Drop-down Menu (0 to 150) |
| 2) What is your height & weight? | Select Imperial or Metric (Dials) |
| 3) What is your biological gender? | M or F or N/A |
| 4) What is your socio-economic class? | Drop-down Menu (Range of Classes) |
| 5) What is your primary ethnicity? | Drop-down Menu (All Ethnicities) |
| 6) What is your secondary ethnicity? | Drop-down Menu (All Ethnicities) |
| 7) What is your level of health literacy? | Drop-down Range (None to Extreme) |
| 8) What are religious food influences? | Drop-down Menu (All Religions) or N/A |
| 9) What is your occupation? | Drop-down Menu (All Occupations) |
| 10) What is your living environment? | Drop-down Menu (Living Environment Types) |

### 1.B. Personal & Demographic Information: Consumer User-Related Inputs

| QUESTIONS: (1-10) | ANSWERS: | WEIGHT RANGE: | START: |
|---|---|---|---|
| 1) What is your current age? | Age: | Range: 10%-20% | 16% |
| 2) What is your height & weight? | Height & Weight: | Range: 11%-20% | 18% |
| 3) What is your biological gender? | Biological Gender: | Range: 7%-16% | 12% |
| 4) What is your socio-economic class? | Socio Status: | Range: 9%-17% | 13% |
| 5) What is your primary ethnicity? | Primary Ethnicity: | Range: 6%-15% | 10% |
| 6) What is your secondary ethnicity? | Secondary Ethnicity: | Range: 2%-7% | 5% |
| 7) What is your level of health literacy? | Health Literacy: | Range: 6%-13% | 9% |
| 8) What are religious food influences? | Religious Food Pref: | Range: 0%-5% | 2% |
| 9) What is your occupation? | Occupation Impacts: | Range: 4%-11% | 7% |
| 10) What is your living environment? | Environment Impact: | Range: 2%-12% | 8% |

| | | |
|---|---|---|
| TOTAL OF 10 INITIAL DYNAMIC QUESTIONS: | Range: 0%-20% | 100% |

### Questions for RNS Input to Assess Food Relevance (Continue to IIA.):

OPT OUT: (OPT out of Any or All Questions; Please fill in any questions that you Choose)
OPT IN: (OPT in to share Any Personal Health History; HIPPA Compliant Confidential Uses)

### II.A. Personal Medical History: Initial 15 Dynamic Questions & Weighted Influence (More Questions Answered, More Relevant RNS FOODSCORE, HIPPA & Regulatory Compliant)

| | ANSWERS; options, opt-ins, |
|---|---|
| QUESTIONS: (1-15) | opt-outs, custom, etc. |
| 1) Do you have any pre-existing medical conditions? | Drop-down Menu |
| | (By Types) |
| 2) Are you currently taking any medications or supplements? | Drop-down Menu |
| | (By Types) |
| 3) Have you experienced any recent changes in your health? | Drop-down Menu |
| | (By Types) |
| 4) Do you have food allergies, sensitivities or intolerances? | Drop-down Menu |
| | (By Types) |
| 5) Current condition/prior diagnoses; any following conditions? | (Select all that apply) |
| a) Diabetes | |
| b) High blood pressure or High cholesterol, other High levels | (Drop-down) |
| c) Celiac disease | |
| d) Inflammatory bowel disease or Other digestive disorders | |
| e) Pre-natal or post-natal or other related concerns | (Drop-down) |
| | Mother and/or Child |
| 6) Are you currently taking any medications? | Drop-down Menu |
| | (By Brand/Class) |
| 7) Do you follow any specific diet or eating pattern? | (Select all that apply) |
| a) Vegetarian or Vegan | |
| b) Pescetarian | |
| c) Gluten-free | |
| d) Mediterranean | |
| e) Low carbohydrates | |
| f) High Protein | |
| g) Paleo, Keto, other | |
| h) Low Fat, Low Sodium | |
| i) Other TBD | |
| 8) Any digestive system surgeries or medical procedures? | No or Drop-down List |
| 9) Any of the following symptoms after eating certain foods? | (Select all that apply) |
| a) Bloating (Drop-down frequency, intensity and triggers) | Drop-down Menu |
| b) Abdominal pain or Acid Reflux (Drop-down paid level and frequency) | |
| c) Diarrhea or Frequent Bowel Movements (Drop-down menu of frequency/intensity) | |
| d) Constipation (Drop-down menu of intensity and frequency) | |
| e) Heartburn (Drop-down menu of intensity score) | |
| f) Nausea or upset stomach (Drop-down menu of intensity, frequency, color) | |
| g) Other TBD (Drop-down list of other side-effects and symptoms) | |
| 10) Have you ever been diagnosed with nutrient deficiencies? | No or Drop-down List |
| 11) Have family history of food-related medical conditions? | Yes or No, If so; |
| | Drop-down List |
| 12) Currently pregnant or breastfeeding? | No or Select Term |
| 13) Have you experienced any weight changes in the past 6 months? | 0 or provide +/− % |
| 14) Do you have any chronic health issues impacting daily life? | Yes or No, If so; |
| | Drop-down Class |
| 15) How do you describe your overall eating habits / dietary pattern? | Drop-down Menu |

### II.B. Personal Medical History: Consumer User-Related Inputs

| | | | |
|---|---|---|---|
| QUESTIONS: (1-15) | ANSWERS: | WEIGHT RANGE: | START: |

| | | |
|---|---|---|
| 1) Do you have any pre-existing medical conditions? | Range: 6%-18% | 11% |
| 2) Are you currently taking any medications or supplements? | Range: 4%-15% | 9% |
| 3) Have you experienced any recent changes in your health? | Range: 2%-12% | 7% |
| 4) Do you have food allergies, sensitivities or intolerances? | Range: 3%-15% | 8% |
| 5) Current condition/prior diagnoses; any following conditions? | Range: 5%-16% | 10% |
| 6) Are you currently taking any medications? | Range: 0%-8% | 3% |
| 7) Do you follow any specific diet or eating pattern? | Range: 3%-15% | 8% |
| 8) Any digestive system surgeries or medical procedures? | Range: 0%-8% | 4% |
| 9) Any of the following symptoms after eating certain foods? | Range: 1%-12% | 6% |
| 10) Have you ever been diagnosed with nutrient deficiencies? | Range: 1%-10% | 5% |
| 11) Have family history of food-related medical conditions? | Range: 1%-8% | 4% |
| 12) Currently pregnant or breastfeeding? | Range: 0%-7% | 3% |
| 13) Have you experienced weight change in the past 6 months? | Range: 4%-17% | 10% |
| 14) Do you have any chronic health issues impacting daily life? | Range: 1%-11% | 6% |
| 15) How you describe your overall eating habits/dietary pattern? | Range: 1%-11% | 6% |
| TOTAL OF 15 INITIAL DYNAMIC QUESTIONS: | Range: 0%-18% | 100% |

### Questions for RNS Input to Assess Food Relevance (Continue to IIIA.):

OPT OUT: (OPT out of Any or All Questions; Please fill in any questions that you Choose)
OPT IN: (OPT in to share Any Personal Health History; HIPPA Compliant Confidential Uses)

### III.A. Family Health History: 10 Initial Dynamic Questions/Weights of Influence (More Questions Answered, More Relevant RNS FOODSCORE, HIPPA and Regulatory Compliant)

| | ANSWERS; options, opt-ins, |
|---|---|
| QUESTIONS: (1-10) | opt-outs, custom, etc. |
| 1) Have you/any close blood relatives had specific chronic diseases? | (Select all that apply) |
| a) Heart Disease (coronary artery disease, heart attacks, and high blood pressure) | |
| b) Type 2 diabetes | |
| c) Cancer (Particularly breast, colon, prostate cancers) | |
| d) Stroke | |
| e) High Cholesterol | |
| f) Osteoporosis | |
| g) Alzheimer's disease / dementia disorders | |
| h) Asthma / other respiratory | |
| i) Hereditary hemochromatosis (Iron Disorders) | |
| 2) Is/Has any family member been diagnosed with Cancer? Age? | Drop-down Menu |
| Please complete all applicable | (By Types/Ages) |
| 3) Are there hereditary conditions or family genetic disorders? | Drop-down Menu |
| Please complete all applicable | (By Types/Ages) |
| 4) Any relatives had multiple cancer diagnoses, same cancer type? | No or Yes; |
| Please complete all applicable | Drop-down Menu |
| | (By Types/Ages) |
| 5) Does anyone in the family have food allergies or intolerances? | No or Yes; |
| | Drop-down Menu |
| 6) What are your maternal and paternal family's ethnic backgrounds? | Drop-down Menus: |
| | Mother/Father |
| 7) Have any family members had/have autoimmune disorders? | No or Yes; |
| | Drop-down Menu |
| 8) Any family history of digestive issues, gastrointestinal diseases? | No or Yes; |
| | Drop-down Menu |
| 9) Have any relatives been diagnosed with nutrient deficiencies? | No or Yes; |
| | Drop-down Menu |
| 10) What are typical eating habits or dietary patterns in our family? | Drop-down Menu |

### III.B. Family Health History:

| | | | |
|---|---|---|---|
| QUESTIONS: (1-10) | ANSWERS: | WEIGHT RANGE: | START: |

| | | |
|---|---|---|
| 1) Have you/close blood relatives had specific chronic diseases? | Range: 8%-22% | 15% |
| 2) Is/Has any family member been diagnosed with Cancer? Age? | Range: 7%-22% | 15% |
| 3) Are there hereditary conditions or family genetic disorders? | Range: 6%-20% | 14% |
| 4) Any relatives had multiple cancer diagnoses, cancer types? | Range: 5%-18% | 12% |
| 5) Does anyone in the family have food allergies or intolerances? | Range: 4%-17% | 10% |
| 6) What is your maternal/paternal family's ethnic backgrounds? | Range: 2%-16% | 9% |
| 7) Have any family members had/have autoimmune disorders? | Range: 2%-15% | 8% |
| 8) Any family history: digestive issues, gastrointestinal diseases? | Range: 0%-13% | 6% |
| 9) Have any relatives been diagnosed with nutrient deficiencies? | Range: 0%-12% | 5% |
| 10) What are typical eating habits or dietary patterns in our family? | Range: 0%-13% | 6% |
| TOTAL OF 10 INITIAL DYNAMIC QUESTIONS: | Range: 0%-22% | 100% |

### Questions for RNS Input to Assess Food Relevance (Continue to IVA.):

OPT OUT: (OPT out of Any or All Questions; Please fill in any questions that you Choose)
OPT IN: (OPT in to share Any Personal Health History; HIPPA Compliant Confidential Uses)
   IV.A. Lifestyle & Behavioral Factors: 10 Initial Dynamic Questions/Influence Weights (More questions answered, more relevant an RNS FOODSCORE; HIPPA regulatory compliant):

| | ANSWERS; options, opt-ins, |
|---|---|
| QUESTIONS: (1-10) | opt-outs, custom, etc. |
| 1) Main health and wellness goals related to your lifestyle? | (Please select all that apply) |
| a) physical fitness | |
| b) reducing stress | |
| c) mental health | |
| d) managing a chronic condition | |
| e) disease prevention | |
| f) fighting a specific disease (Drop-down menu) | |
| g) other goals (Drop-down menu) | |
| 2) How would you describe your current eating habits? | Drop-down Menu (By Types) |
| a) regular meals | |
| b) frequent snacking | |
| c) emotional eating | |
| d) irregular/other eating patterns (Drop-down options) | |
| e) majority drive-thru convenience | |
| 3) Do you experience barriers to maintaining a healthy diet? Please complete all applicable | |
| a) time constraints | |
| b) financial limitations | |
| c) lack of cooking skills | |
| d) limited access to healthy foods | |
| e) health issues & limitations (Drop-down menu) | |
| f) medication side-effects (Drop-down menu) | |
| g) lifestyle constraints (e.g., Frequent Travel, other?) | (Drop-down menu) |
| 4) Consumption of meals prepared outside of home | (e.g., restaurants, takeout) |
| | Drop-down Menus: |
| | # and Frequency |
| 5) What role does stress play in your eating habits? | Stress Influence? |
| | Drop-down Menus: |
| | Degree of Impact (% meter) |
| a) stress-induced overeating | |
| b) loss of appetite | |
| c) no impact | |
| 6) How much sleep do you get each day/night? Sleep Impact? | Drop-down Menus: |
| | Time Meter (Dial) |
| 7) How much and what type of physical activity do you get daily? | Drop-down Menus: |
| | Time/Intensity |
| 8) What habits or routines influence your food choices or routines? | Drop-down Menu: Habit List |
| a) meal prepping | |
| b) dining out | |
| c) social eating | |
| d) other (list) | |
| 9) Primary motivations for making dietary changes? | Drop-down Menus: |
| a) health improvement | |
| b) weight loss | |
| c) ethical reasons | |
| d) other? | |
| 10) Confidence in ability to make/maintain dietary changes? | Drop-down Menu: |
| | Confidence % Meter |

### IV.B. Lifestyle & Behavioral Factors:

| | | | |
|---|---|---|---|
| QUESTIONS: (1-10) | ANSWERS: | WEIGHT RANGE: | START: |

| 1) Main health and wellness goals related to your lifestyle? | Range: 3%-11% | 7% |
|---|---|---|
| 2) How would you describe your current eating habits? | Range: 2%-13% | 9% |
| 3) Do you experience barriers to maintaining a healthy diet? | Range: 10%-20% | 12% |
| 4) Consumption of meals prepared outside of home | Range: 10%-21% | 15% |
| 5) What role does stress play in your eating habits? Stress? | Range: 3%-13% | 7% |
| 6) How much sleep do you get each day/night? Sleep Impact? | Range: 5%-13% | 8% |
| 7) How much, what type of physical activity do you get daily? | Range: 10%-18% | 13% |
| 8) What habits influence your food choices or routines? | Range: 3%-11% | 5% |
| 9) Primary motivations for making dietary changes? | Range: 5%-14% | 11% |
| 10) Confidence in ability to make/maintain dietary changes? | Range: 8%-16% | 13% |
| TOTAL OF 10 INITIAL DYNAMIC QUESTIONS: | Range: 2%-21% | 100% |

### Questions for RNS Input to Assess Food Relevance (Continue to VA.):

OPT OUT: (OPT out of Any or All Questions; Please fill in any questions that you Choose)
OPT IN: (OPT in to share Any Personal Health History; HIPPA Compliant Confidential Uses)
   V.A. Mental Health & Wellbeing: 5 Initial Dynamic Questions/Weights of Influence (More questions answered, more relevant RNS FOODSCORE, HIPPA and regulatory compliant)

| | ANSWERS; options, opt-ins, |
|---|---|
| QUESTIONS: (1-5) | opt-outs, custom, etc. |
| 1) Does your current mental health influence eating habits? | (Select all that apply) |
| a) positive outlook | |
| b) depression | |
| c) anxiety | |
| d) no issues | |
| e) proactive healthy eating | |
| f) comfort foods | |
| g) impulse snacking | |
| h) skipping meals | |
| i) binge eating | |
| j) starving | |
| k) never thin enough | |
| 2) What role does stress play in your dietary decisions? | (Select all that apply) |
| a) tendency to overeat | |
| b) tendency to undereat | |
| c) impulse sweet treats | |
| d) irregular eating patterns | |
| e) late night eating binges | |
| f) secret eating | |
| g) self-image eating | |
| h) eating to benefit others | |
| i) calorie obsessed | |
| j) normal, no issues | |
| k) skip then binge patterns | |
| 3) How does your mood fluctuate with changes in your diet? | Drop-down Menu: Impact |
| 4) Any change in mental clarity/focus based on what you eat? | Drop-down Menus: Food |
| a) improve function | |
| b) impair function | |
| c) guilt | |
| d) optimism | |
| e) positive outlook | |
| f) negative outlook | |
| g) stability | |
| 5) Confidence in dietary changes to support mental health? | Drop-down Menu: |
| | Confidence % |

### V.B. Mental Health & Wellbeing:

| | | | |
|---|---|---|---|
| QUESTIONS: (1-5) | ANSWERS: | WEIGHT RANGE: | START: |

| | | |
|---|---|---|
| 1) Does your current mental health influence eating habits? | Range: 15%-28% | 22% |
| 2) What role does stress play in your dietary decisions? | Range: 13%-27% | 22% |
| 3) How does your mood fluctuate with changes in your diet? | Range: 14%-26% | 20% |
| 4) Any change in mental clarity, focus based on what you eat? | Range: 13%-22% | 18% |
| 5) Confidence in dietary changes to support mental health? | Range: 16%-22% | 18% |
| TOTAL OF 5 INITIAL DYNAMIC QUESTIONS: | Range: 13%-28% | 100% |

Questions for RNS Input to Assess Food Relevance (Continue to VLA.):
OPT OUT: (OPT out of Any or All Questions; Please fill in any questions that you Choose)
OPT IN: (OPT in to share Any Personal Health History; HIPPA Compliant Confidential Uses)
   VI.A. Environmental & Social Factors: 10 initial dynamic Questions/Weights of Influence. (The more questions answered, the more relevant an RNS FOODSCORE, HIPPA and regulatory compliant)

| | ANSWERS; options, opt-ins, |
|---|---|
| QUESTIONS: (1-10) | opt-outs, custom, etc. |
| 1) Does local food environment (grocery stores, farmers' markets) influence | Yes, or No?; Drop-down |
| your food choices? | Selection |
| 2) Do social interactions affect your eating patterns? | Yes, or No?; Low to High % Dial |
| e.g., Family, Friends, Co-workers, Teammates, Spouse, Children, Trainor | |
| 3) Do economic factors, such as food prices and your budget, influence the types and quality of | Drop-down Menu: Impact Range |
| foods you consume? | |
| 4) To what extent do cultural norms and traditions shape your food | Drop-down Menu: Impact Range % |
| preferences and choices? | If 50%+, Drop-down Cultural Foods |
| 5) How accessible are healthy food options to you? | Drop-down Menu: Very to Not |
| Does this affect and impact your diet? | Yes, or No; Good to Bad Scale. |
| 6) Do you have reliable transportation to access a variety of food outlets? | Yes, or No; Distance, convenience? |
| 7) Frequency of eating out at restaurants or order takeout; Does this | Drop-down Menu: Frequency Meter |
| affect your nutritional intake? | Drop-down: Low-High Impact |
| 8) Does food marketing or advertising impact your food purchasing habits? | Yes, or No; Impact Drop- |
| | down % |
| 9) How do your work or school environments affect eating habits? | Options, Time Constraints, Quality? |
| 10) Do community/social events influence your food choices? | Yes, or No; Bad to Good Meter |

### VI.B. Environmental & Social Factors:

| | | | |
|---|---|---|---|
| QUESTIONS: (1-10) | ANSWERS: | WEIGHT RANGE: | START: |

| | | |
|---|---|---|
| 1) Does your food environment (grocery stores, farmers' markets) | Range: 15%-26% | 21% |
| influence your food choices? | | |
| 2) Do your social interactions affect your eating patterns? | Range: 11%-20% | 15% |
| 3) Do economic factors, such as food prices and your budget, | Range: 12%-23% | 18% |
| influence the types and quality of foods you consume? | | |
| 4) Do cultural norms/traditions shape food preferences/choices? | Range: 8%-15% | 10% |
| 5) How accessible are healthy food options to you? | Range: 10%-16% | 12% |
| 6) Do you have reliable transportation, access to food outlets? | Range: 4%-10% | 6% |
| 7) Frequency of eating out at restaurants or order takeout; | Range: 4%-9% | 6% |
| Does this affect your nutritional intake? | | |
| 8) Does food marketing/advertising impact your food purchasing? | Range: 3%-8% | 5% |
| 9) How do your work/school environments affect eating habits? | Range: 2%-8% | 4% |
| 10) Do community or social events influence your food choices? | Range: 1%-8% | 3% |
| TOTAL OF 10 INITIAL DYNAMIC QUESTIONS: | Range: 1%-26% | 100% |

### Questions for RNS Input to Assess Food Relevance (Continue to VIA.):

OPT OUT: (OPT out of Any or All Questions; Please fill in any questions that you Choose)
OPT IN: (OPT in to share Any Personal Health History; HIPPA Compliant Confidential Uses)

VII.A.Health Goals & Concerns: 20 initial dynamic Questions/Weights of Influence (More questions answered, more relevant RNS FOODSCORE, HIPPA and regulatory compliant)

| | ANSWERS; options, opt-ins, |
|---|---|
| QUESTIONS: (1-20) | opt-outs, custom, etc. |
| 1) What are your primary health goals? | Drop-down Selection |
| (e.g., weight loss, muscle gain, improved energy levels, improved mental health, etc.) | |
| 2) Do you have any specific dietary restrictions or allergies? | Yes, or No? |
| | Drop-down Selection |
| 3) Are you managing chronic diseases or health conditions? | Yes, or No? |
| (e.g., diabetes, hypertension, heart disease) | Drop-down Selection |
| 4) What is your target blood pressure range? | Drop-down Selection |
| 5) Do you have specific cholesterol level goals? | Yes, or No? |
| | Drop-down Selection |
| 6) Are you aiming to improve your gut health or digestion? | Yes, or No? |
| | Drop-down Options |
| 7) How many meals do you eat in a day? | Drop-down Selection |
| 8) What types of foods do you currently consume frequently? | Drop-down Selection |
| 9) How often do you eat fruits and / or vegetables? | Yes, or No? |
| | Drop-down Selections |
| 10) Do you follow a specific diet plan? If so, which one? | Yes, or No? |
| | Drop-down Selection |
| 11) What is your level of physical activity? | Drop-down Selection |
| 12) How often do you consume processed or fast food? | Drop-down Selections |
| 13) How do you track your nutrition and dietary intake? | Drop-down Options |
| 14) What are your challenges for health and nutrition goals? | Drop-down Options |
| 15) Are you taking medications that affect diet/nutrient absorption? | No, or Drop-down Menu |
| 16) Do you have specific nutrient deficiencies you want addressed? | No, or Drop-down Selection |
| 17) Are you concerned about food intolerances or sensitivities? | No, or Drop-down Selection |
| 18) How does stress impact your eating habits? | Drop-down Selection/Meter |
| 19) Do you have any specific fitness performance goals? | No, or Drop-down Selection |
| 20) Are you trying to work key superfoods/supplements into diet? | No, or Drop-down Selection |

### VII.B. Health Goals & Concerns:

| | | | |
|---|---|---|---|
| QUESTIONS: (1-20) | ANSWERS: | WEIGHT RANGE: | START: |

| | | |
|---|---|---|
| 1) What are your primary health goals? | Range: 8%-18% | 11% |
| 2) Do you have any specific dietary restrictions or allergies? | Range: 8%-15% | 9% |
| 3) Are you managing chronic diseases or health conditions? | Range: 9%-16% | 9% |
| 4) What is your target blood pressure range? | Range: 4%-10% | 5% |
| 5) Do you have specific cholesterol level goals? | Range: 4%-10% | 5% |
| 6) Are you aiming to improve your gut health or digestion? | Range: 3%-9% | 4% |
| 7) How many meals do you eat in a day? | Range: 2%-8% | 4% |
| 8) What types of foods do you currently consume frequently? | Range: 4%-11% | 6% |
| 9) How often do you eat fruits and / or vegetables? | Range: 4%-10% | 7% |
| 10) Do you follow a specific diet plan? If so, which one? | Range: 2%-8% | 5% |
| 11) What is your level of physical activity? | Range: 3%-9% | 6% |
| 12) How often do you consume processed or fast food? | Range: 2%-8% | 4% |
| 13) How do you track your nutrition and dietary intake? | Range: 1%-4% | 3% |
| 14) What are your challenges for health and nutrition goals? | Range: 1%-5% | 3% |
| 15) Are you taking medications that affect diet/nutrient absorption? | Range: 3%-8% | 4% |
| 16) Do you have specific nutrient deficiencies you want addressed? | Range: 2%-7% | 4% |
| 17) Are you concerned about food intolerances or sensitivities? | Range: 2%-6% | 4% |
| 18) How does stress impact your eating habits? | Range: 1%-5% | 3% |
| 19) Do you have any specific fitness performance goals? | Range: 1%-5% | 2% |
| 20) Are you trying to work key superfoods/supplements into diet? | Range: 0%-4% | 2% |
| TOTAL OF 20 INITIAL DYNAMIC QUESTIONS: | Range: 0%-18% | 100% |

### END OF SURVEY SUMMARY OPTION: Disclosure Confirmation

OPT OUT: (OPT out of Any or All Questions; Please fill in any questions that you Choose)
OPT IN: (OPT in to share Any Personal Health History; HIPPA Compliant Confidential Uses)

In summary, the above sample Consumer User Survey is a dynamic, ever-evolving AI-enabled process and systematic environment, created to assess all available data, research and inputs (directly or indirectly) impacting the past, current or future health of individual consumers (users), which can be addressed or affected through food, medication and nutrition consumption.

The IRX FOODSCORE creates a relevant nutrition score (RNS) for any existing or prospective product(s) which speaks specifically to purposeful consumption of said product and its likely health impact upon designated users. Initially, there are twelve (12) distinct USERS or groups defined, each having its own commensurate and correlating surveys and questions, which likewise result in the creation of relevant nutrition scores for purposes and health uses defined.

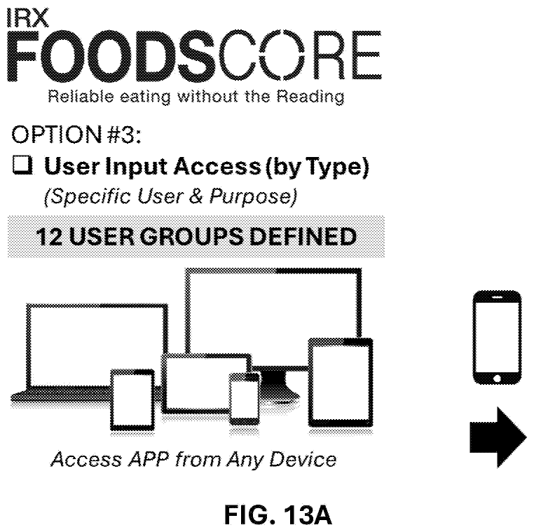

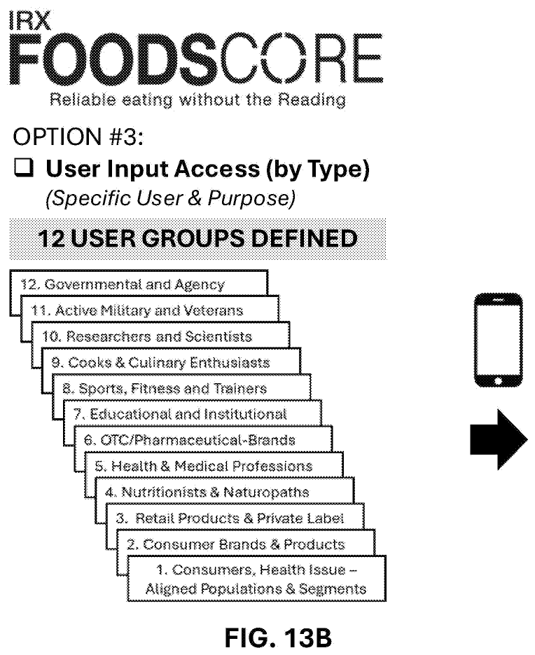

FIG. 13A-B illustrate how any device can be used to access the app and address any of the 12 initial user groups.

In summary, the Systems and Methods for Artificial Intelligence Analyses and Scoring of Consumables disclosed herein will include the following primary features as well as prominent design and operational features:
1. An integrated IRX FOODSCORE ecosystem according to the present invention, comprising:
   (a) advanced artificial intelligence (AI)-empowered system, processing platform and data refinery, which dynamically sources, scientifically applies and purposefully analyzes the entire authoritative body of human knowledge, encompassing any and all research, documentation and disparate data relevant to health influencers, impacts or impositions, and pertinent to any and all possible health issues, impairments or opportunities encountered, or for any defined health issues incurred by or objective outcomes defined by user designates, accessible by any interface device.
   (b) advanced artificial intelligence (AI)-enabled methodologies, dynamic algorithms, scalable scoring techniques, risk profiling and compliance assessments that transform the authoritative body of human knowledge, research, documentation and disparate data into seamlessly integrated, user-aligned, actionably relevant and purposefully planned consumption of a food, beverage or consumable in any form, necessary to strategically support, tactically target and distinguish dietary drivers and efficacies, which are population-specific or personally purposeful, actionable and functional in support of user-aligned or defined objective health outcomes, optimized nutrition or issue-centric opportunities, applied health & wellness and dietary applications.
   (c) advanced artificial intelligence (AI)-enriched software, evolutionary and interactive applications, leveraging the aforementioned integrated AI-empowered system and applied, generative methodologies to deliver user-aligned, actionable and health-relevant consumable supply chains' content through smart devices, appliances or apparatuses, mobile applications, shopping assistants, e-Health platforms, dietary technologies, computer programs, cloud-based service portals (e.g., SaaS, others, etc.), wireless or hard-wired accessories, and whether directly accessed by, or indirectly defined on behalf of user-specific individuals, groups or communities, either proactively or reactively to support and optimize realized health outcomes.
2. The ecosystem according to the present invention, wherein user input is received explicitly or implicitly by survey or inputs of actual health records, inferences, or insights from medical observations or behaviors, required to define or refine user-aligned health relevance filters and content criteria.
3. The ecosystem according to the present invention, wherein user-aligned, defined or relevant health, dietary or consumable data may be provided, collected or distributed directly to or through said ecosystem tools and technologies as available, or directly, indirectly or alternatively through dependent 3^{rd} party data collection or observation tools, technologies, platforms, and devices used for delivering user-aligned or specified and relevant AI-enriched consumable and nutrition outputs, insights and health impacts, proactively, situationally or reactively, not limited by or to:
   (a) AI-powered nutrition apps and health tracking platforms: January AI, Nutrigenomix, That Clean Life, BetterMeal.AI, MealMate, Mealmind, Foodvisor, EatLove, Nutrino, GenoPalate, Nutripal, The Drop, FITBIT, Apple Watch, and others, etc.
   (b) AI-driven food analysis tools: Image recognition and food scanning technologies, Barcode scanners for packaged foods, Voice and text search capabilities, and others, etc.
   (c) Wearable devices and sensors: Continuous glucose monitors (CGMs), Heart rate monitors (HRMs), Smartwatches with health tracking features, and others, etc.
   (d) Genetic testing platforms: DNA analysis tools for personalized nutrition recommendations
   (e) Machine learning (ML) algorithms: Predictive analytics for blood sugar levels, Food recognition and nutrient composition analysis, Personalized meal planning and recipe generation, and others, etc.
   (f) Natural language (NL) processing: AI chatbots, interactive AI and virtual health coaches, and others, etc.
   (g) Data integration platforms: Systems that combine multiple data sources (e.g., food logs, activity data, genetic information, etc.) and others, etc.
   (h) Cloud-based data storage and processing: For handling large dynamic, disparate datasets and complex computations, and other activities, etc.
   (i) Alternative devices: On-site computers, tablets, i-pads, portals, gateways, other hardware or fixed site collection devices, and others, etc.
   (i) Mobile devices: Smartphones for app usage, virtual engagements, data collection and inputs, smart shopping carts, e-carts, on-site, in-store or on-floor devices, and others, etc.
   (j) IoT-enabled kitchen appliances: Smart refrigerators, smart scales, connected cooking devices, virtual pantries, active AI area consumption, tracking and dietary tools, others, etc.
4. The ecosystem according to the present invention, wherein certain scientific databases and research institutions provide dependent data necessary to deliver user-relevant health impacts, functional and actionable nutrition insights, expected food effects and efficacies of consumption:
   (a) PubMed: a dynamic, evolving and comprehensive 37 million citation database providing access to full content research and studies on PubMed Central and publisher websites, including but not limited to biomedical literature, health impacts and nutrition studies.
5. The ecosystem according to the present invention, wherein certain scientific databases and research institutions provide dependent data related to global health, including but not limited to World Health Organization (WHO) Databases:
   (a) Global Database on Child Growth and Malnutrition.
   (b) Evaluations of the Joint FAO/WHO Expert Committee on Food Additives (JECFA).
   (c) Global Environment Monitoring System (GEMS), other environmental health influencers.
   (d) Food Contamination Monitoring and Assessment Programme.
   (e) Global database on the Implementation of Food and Nutrition Action (GIFNA)
   (f) WHO Global Data Bank on Infant and Young Child Feeding.
6. The ecosystem according to the present invention, wherein certain research studies provide dependent data necessary to deliver user-relevant health impacts, purposeful nutrition insights, and expected food effects & efficacies of consumption; e.g., Global Burden of Disease (GBD) Study: Providing comprehensive data, dietary risks, health impacts across 195 countries.
7. The ecosystem according to the present invention, wherein certain global databases provide dependent data necessary to deliver user-relevant health and nutrition impacts, expected food effects and efficacies of consumption, and specifically Food and Agriculture Organization (FAO) Databases: FAOSTAT, providing free access to food and agriculture data in over 245 countries & territories.
8. The ecosystem according to the present invention, wherein certain governmental agency databases provide dependent data necessary to deliver user-relevant health and nutrition impacts, functional insights, expected food effects and efficacies of consumption, and specifically as an example, the USDA Food Data Central: Offering detailed nutritional information on a wide range of foods.
9. The ecosystem according to the present invention, wherein certain International Research Projects and Reports provide dependent data necessary to deliver user-relevant health impacts, functional and actionable nutrition insights, expected food effects and efficacies of consumption, not limited to:
   (a) Global Nutrition Report: Comprehensive food system, nutrition analyses, health outcomes.
   (b) EAT-Lancet Commission: Science-based healthy diet targets; sustainable food production.
   (c) Nutrition Landscape Information System (NLiS): Web-based tool providing nutrition and nutrition-related health and development data.
   (d) Nature Journal Studies: Quality research, global diets, environmental and health impacts.
   (e) Lancet Studies: Offering in-depth analyses of global dietary risks and health impacts.
10. The ecosystem according to the present invention, wherein certain Academic Institutions and Libraries provide dependent data necessary to deliver user-relevant health impacts, as well as functional and actionable nutrition insights, expected food effects and efficacies of consumption, such as:
   (a) University Research Guides, not limited to: LSU Libraries' Research Guide for Nutrition & Food Sciences, which provides access to specialized databases and resources.
11. The ecosystem according to the present invention, wherein implementation of methodologies and dynamic algorithms are dependent upon utilization and incorporation of advanced artificial intelligence (AI) languages, tools, frameworks and technologies, including but not limited to:
   (a) Programming Languages: Python, R, Java, C++, Julia, others, etc.
   (b) Deep Learning (DL) Frameworks: TensorFlow, PyTorch, Keras, others, etc.
   (c) Machine Learning Libraries (ML): scikit-learn, NumPy, SciPy, Pandas, others, etc.
   (d) Natural Language Processing: NLTK, spaCy, Gensim, others, etc.
   (e) Computer Vision; OpenCV, SimpleCV, others, etc.
   (f) Data Visualization: Matplotlib, ggplot2 (for R), others, etc.
   (g) Cloud Platforms: Google Cloud AI, Amazon Web Services (AWS) AI, Microsoft Azure AI or others, etc.
   (h) Model Deployment & Scalability: Docker, Kubernetes, others, etc.
   (i) Version Control: Git, other, etc.
   (j) Integrated Development Environment (IDEs): Jupyter Notebooks, PyCharm, RStudio, etc.
12. The ecosystem according to present invention, wherein secure scalable deployment of methodologies, user-aligned smart content and dynamic deliverables are dependent upon the incorporation and utilization of certain AI-powered tools and technologies, not limited to:
   (a) Programming Languages: Python, R, JavaScript, and others, etc.
   (b) AI Machine Learning (ML) Frameworks: TensorFlow, PyTorch, scikit-learn, others, etc.
   (c) Natural Language (NL) Processing: NLTK, spaCy, and others, etc.
   (d) Data Analysis and Visualization: Pandas, Matplotlib, D3.js, and others, etc.
   (e) Cloud Platforms: Google Cloud AI, Amazon Web Services (AWS), Microsoft Azure, etc.
   (f) Mobile Development: React Native, Flutter, and others, etc.
   (g) AR/VR (Augmented/Visual Reality) Development: Unity, Unreal Engine, and others, etc.
   (h) Database Management: MongoDB, PostgreSQL, and others, etc.
   (i) API Integration: RESTful APIs, GraphQL, and others, etc.
   (j) Version Control: Git, and others, etc.
   (k) Deployment and Scaling: Docker, Kubernetes, and others, etc.
   (l) User Authentication and Security: OAuth 2.0, JSON Web Tokens (JWT), and others, etc.
13. The ecosystem according to the present invention, wherein advanced artificial intelligence (AI)-enriched software, evolutionary and interactive applications are utilized to directly or indirectly deliver user-relevant health impacts, functional and actionable nutrition insights, expected food effects and efficacies of conspicuous consumption, available to or through, but not limited by:
   (a) Smart Health and Nutrition Solutions: Personalized Meal Planning Systems, Real-Time Dietary Monitoring and Feedback Applications, AI-Powered Nutritional Recommendation Engines, Interactive Generative AI or Virtual Health Assistants, Augmented Reality (AR) Nutrition Education Tools, Virtual Reality (VR) Diet Simulation Environments, others, etc.
14. The ecosystem according to the present invention, wherein advanced artificial intelligence (AI)-enriched software, evolutionary and interactive applications are utilized to directly or indirectly deliver user-relevant health impacts, functional and actionable nutrition insights, expected food effects & efficacies of conspicuous consumption, is dependent upon 3^{rd} party technologies, tools and security protocols to develop and deploy robust, scalable, and secure AI-enabled applications and software for user-aligned health and nutrition purposes, updated ongoing with latest security practices and regulatory requirements to develop or maintain applications, not limited to:
   (a) Programming Languages: Python used as primary language for AI and machine learning (ML) development, JavaScript used for web-based front-end development, Java used for cross-platform compatibility and integration with existing systems, and others, etc.
   (b) AI and Machine Learning (ML) Frameworks: TensorFlow or PyTorch used for developing and training AI models, scikit-learn used for implementing machine learning (ML) algorithms, Natural Language Processing (NLP) libraries used for processing text inputs and generating responses, and others, etc.
   (c) Mobile App Development: React Native or Flutter used for cross-platform mobile app development, Swift (iOS) and Kotlin (Android) used for native mobile app development as required, and others, etc.
   (d) Web Development: React.js or Vue.js used for building interactive web interfaces, ode.js used for server-side development, or others, etc.
   (e) Database Management: PostgreSQL or MongoDB used for storing and managing user data, dietary information, and health records, and others, etc.
   (f) Cloud Services: AWS, Google Cloud or Azure used for scalable, global APP deployment, including Hybrid Cloud, which involves a mix of public/private clouds and on-premises resources, while cloud-edge computing is a hybrid model that processes data at the network's edge to reduce latency for real-time applications, such as from IoT devices. The two are often combined: a hybrid cloud can include edge locations as part of its mixed environment, enabling applications to leverage both high-performance edge processing and scalable cloud resources for different tasks.
   (g) Security Protocols and Data Encryption: Implement AES-256 encryption for data at rest and TLS for data in transit; Secure key management practices to safeguard encryption keys.
   (h) Authentication and Authorization: Implementation of multi-factor authentication (MFA) for user accounts; Use of OAuth2 or JWT for secure API authentication; Applied principle of least privilege for access controls, and others, etc.
   (i) Compliance with Regulations: Ensuring compliance with HIPAA (for US) and GDPR (for EU) regulations for handling health data, and other country compliance standards, etc.
   (j) Implementation of agency or country-required features for user consent, data access, and the “right to be forgotten” as required by GDPR, and any and all other required standards.
   (k) Secure Development Practices: Following OWASP Mobile Application Security Checklist; Conducting regular security audits and penetration testing; Implementing input validation and sanitization to prevent injection attacks, and others, etc.
   (l) AI-Specific Security Measures: Customizing general and generative AI architecture with built-in security features; Implementing robust prompt handling and input sanitization for AI interactions; Establishing an AI-specific threat intelligence feed, and others, etc.
   (m) Consideration of User Interface and Experience: Implementing data masking for sensitive information on UI fields; Designing an intuitive interface for food logging and nutritional analyses, and others, etc.
   (n) Integrating with External Devices: Developing APIs for integration for wearable devices, IoT-enabled kitchen appliances and personal shopping carts and technologies, and others, etc.
   (o) Scalability and Performance: Using containerization (e.g., Docker) and orchestration (e.g., Kubernetes) for scalable deployments; Implementing caching mechanisms and content delivery networks (CDNs) for improved performance, and others, etc.
   (p) Continuous Integration/Continuous Deployment (CI/CD): Setting up automated testing and deployment pipelines for regular updates and security patches, and other activities, etc.
   (q) Data Analytics and Reporting: Implementing data visualization libraries (e.g., D3.js or Chart.js) for presenting nutritional insights; Using big data processing tools (e.g., Apache Spark) for handling large-scale data analysis, and others, etc.

The Systems and Methods for Artificial Intelligence Analyses and Scoring of Consumables shown in the drawings and described in detail herein disclose arrangements of elements of particular construction and configuration for illustrating preferred embodiments of structure and method of operation of the present application. It is to be understood, however, that elements of different construction and configuration and other arrangements thereof, other than those illustrated and described may be employed for providing the Systems and Methods for Artificial Intelligence Analyses and Scoring of Consumables in accordance with spirit of this disclosure, and such changes, alternations and modifications as would occur to those skilled in the art are considered to be within the scope of this design as broadly defined in the appended claims.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. For example, one portion of one of the embodiments described herein can be substituted for another portion in another embodiment described herein. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can”, “could”, “might”, or “may”, unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z”, unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately”, “about”, “generally”, and “substantially” as used herein represent a value, amount, or characteristic close to the stated value, amount, or characteristic that still performs a desired function or achieves a desired result. For example, the terms “approximately”, “about”, “generally”, and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Further, the purpose of the foregoing abstract is to enable the U.S. Patent and Trademark Office, foreign patent offices worldwide and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

1. A computer implemented artificial intelligence (AI)-enabled data refinery system comprising at least one processor and a non-transitory computer-readable memory storing instructions that, when executed by the at least one processor, cause the data refinery system to perform AI-based analyzation, scoring, and reporting of consumables, the data refinery system comprising:
a) a data ingestion component implemented by said at least one processor and configured to collect, validate, and standardize disparate input data from a plurality of structured and unstructured sources;
b) a data preprocessing component, operatively connected to and in communication with said ingestion component implemented by said at least one processor and configured to prepare, convert, integrate, and aggregate input data into datasets suitable for downstream processing;
c) a dynamic analyzation and modeling application structured to employ AI infrastructures to dynamically generate Relevant Nutrition Scores (RNS) and Relevant Risk Scores (RRS) through adaptive, iterative interactions with user-defined parameters in the generation of user-aligned and purpose-aligned information for personalized nutrition and consumables related recommendations, reports, and notifications based on the analyzed data;
d) a report generating application and output interface configured to generate, format, and deliver real-time reports, analytics dashboards, and personalized nutrition and consumables recommendations to users through cross-platform interfaces; and
e) an AI organization, feedback, and training component implemented by said at least one processor and configured to monitor data refinery system workflows, manage data refinery task execution, and incorporate user and system feedback to continuously improve data refinery data processing and data analytics performance through machine learning (ML) and deep learning (DL) techniques, including neural networks and large language model (LLM)-based natural language processing;
wherein the AI-enabled data refinery system continuously transforms, incorporates and integrates diverse input data into structured formats, enabling AI-based and adaptive health-driven assessments, consumables formulations, user-aligned purposes and personalized nutrition recommendations based on user defined and specified inputs, and user objective and subjective consumable products, health queries, and real-time physiological feedback; and
further wherein the AI-enabled data refinery system dynamically updates Relevant Nutrition Scoring (RNS) and Relevant Risk Scores (RRS) and recommendations in response to existing, new and speculative consumables and target user data, securely maintaining encrypted health profiles, and organizing information by creating data points and building connections between them that link consumption patterns and published research to specified, aligned and objective health outcomes of actual and intended users.

2. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, wherein said data ingestion module is further configured to:
a) receive input data from user devices, including smartphones, tablets, computers, and wearable sensors;
b) interface with third-party application programming interfaces (APIs) for real-time acquisition of health, nutrition, and demographic data;
c) access and retrieve data from external databases, including governmental, institutional, consumable, pharmacological, pharmaceutical, nutritional and commercial repositories; and
d) receive continuous physiological data from internet-of-things (IoT) devices and health sensors.

3. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, wherein said data preprocessing engine is further configured to:
a) perform automated data cleansing, including removal of duplicates and correction of inconsistencies;
b) normalize and harmonize input data using statistics, machine learning (ML), and deep learning (DL) based normalization and harmonization techniques including z-score normalization, min-max scaling, and neural network-based data harmonization;
c) transform input data into standardized formats compatible with downstream analytics applications;
d) extract characteristics from input data, including identification of key nutritional, demographic, medical, and behavioral variables; and
e) assign quality scores to input data based on source reliability, completeness, recency, and frequency, and to prioritize higher-quality data for analytics.

4. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, wherein said analyzation and modeling application and output interface is further configured to employ existing AI infrastructure to generate Relevant Nutrition Scores (RNS) and Relevant Risk Scores (RRS) and is further configured to:
a) apply adaptive machine learning (ML) algorithms that update model parameters in real time in response to dynamically applied data inputs;
b) employ deep learning (DL) models for pattern recognition in multi-dimensional nutritional and health datasets; and
c) utilize machine learning (ML) models that optimize recommendations based on user feedback and real-world outcomes.

5. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, wherein said analyzation and modeling application is configured to employ existing AI infrastructure to generate Relevant Nutrition Scores (RNS) and Relevant Risk Scores (RRS) and is further structured to:
a) include a real-time compliance certification module that validates nutrition claims and recommendations against regulatory standards from multiple regulatory agencies and jurisdictions;
b) generate real-time scoring, reports and user-insights, making each context transparent, and detailing, explaining and validating all factors and data sources influencing each Relevant Nutrition Score (RNS), Relevant Risk Score (RRS) and recommendations;
c) customize its recommendations, reports, and notifications according to the type of vantage point, intended user and benefactor, including consumers, product manufacturers, healthcare professionals, government agencies and researchers; and
d) customize and evaluate contextual what-if dietary scenarios via probabilistic sampling methods comprising genomic profiling, microbiome sequencing data, and real-time wearable-derived health markers.

6. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, wherein the report generating application and output interface is further configured to:
a) deliver reports and analytics via web-based and desktop interfaces;
b) deliver reports and analytics via mobile applications and IoT devices;
c) deliver reports and analytics through API endpoints for integration with third-party platforms and portals;
d) generate customized reports, dashboards, and user-interfaces (UIs) aligned to user roles, including consumers, clinicians, researchers, and administrators;
e) include optimized nutrient ratios and impact assessments derived from temporal analyses of consumable interactions, coupled with real-time intervention protocols;
f) generate comparative risk profiling via Relevant Risk Scores (RRS), assessing health risk tolerance probabilities; and
g) generate dynamically adaptive alerts when real-time consumable intake data indicates deviation from user-defined thresholds based on established nutritional guidelines.

7. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, further wherein:
a) user profiles are dynamically updated based upon ongoing data inputs, behavioral patterns, and feedback, enabling real-time adjustment of recommendations and risk assessments;
b) all data transactions and modifications follow global data standards to ensure data provenance, system transparency, and regulatory compliance;
c) the computer-implemented AI-enabled data refinery system includes mechanisms for obtaining, managing, and revoking user consent for data collection, processing, and retention, in compliance with applicable data privacy laws; and
d) the computer-implemented AI-enabled data refinery system employs secure data-access protocols, encryption, and error-handling mechanisms to enable robust internal-external interoperability.

8. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, further wherein:
a) said data preprocessing engine uses automated methods to select and aggregate incoming data from existing and real-time data sources, so the computer-implemented AI-enabled data refinery system can process information more efficiently;
b) said data preprocessing engine uses artificial intelligence (AI), machine learning (ML), deep learning (DL), and large language models (LLMs) search techniques to quickly find matching patterns in databases; and
c) said dynamic analyzation and modeling application comprises machine learning (ML) and deep learning (DL) models that adjust nutrient recommendations based on biochemical markers and generate alerts indicating that nutritional absorption patterns exceed defined thresholds.

9. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, further wherein:
a) said data preprocessing component includes external nutrition taxonomies and ontologies that are integrated for cross-system data normalization and harmonization;
b) said dynamic analyzation and modeling application comprises an analytics orchestration engine configured to execute advanced search protocols on specialized computing units to identify multi-dimensional nutrient-deficiency patterns in population-level datasets and to optimize matching between user health parameters and consumable phytochemical profiles; and
c) deep learning (DL) models are trained on consumable-health and pharmaceutical-health interaction matrices accounting for age, gender, drug interactions, and metabolic states, with feedback loops triggered by biochemical marker deviations from defined thresholds.

10. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, further wherein:
a) the computer implemented AI-enabled data refinery system architecture is modular, supporting plug-in components for third-party analytics, data sources, and reporting modules;
b) the computer implemented AI-enabled data refinery system supports deployment on cloud, cloud-edge computing, hybrid cloud, private and public cloud, and on-device infrastructures to optimize performance and accessibility; and
c) the computer implemented AI-enabled data refinery system supports Anything as a Service (XaaS) including Infrastructure as a Service (IaaS), Platform as a Service (PaaS), Back-up as a Service (BaaS), Software as a Service (SaaS), Back-end as a Service (BaaS), Desktop as a Service (DaaS).

11. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, further comprising functionality that enables the computer implemented AI enabled a data refinery system to update and improve its recommendations for all users by learning from feedback, patterns, and results across many users' data, while keeping each user's personal information private and secure, consistent with governmental privacy policies and global data protection standards, and the computer implemented AI enabled a data refinery system further includes an isolated testing environment for evaluation of new algorithms, features, and data sources.

12. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, wherein said analyzation and modeling application is configured to utilize algorithms and models including clustering, classification, regression, and natural language processing for analyzing and interpreting structured and unstructured health, nutrition, and consumable data.

13. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, wherein said analyzation and modeling application is configured to calculate a Relevant Nutrition Score (RNS) utilizing the following equation:
$$R N S = {\sum }_{i = 1}^{9} ( {δ}_{i} \times {G}_{i} )$$
where:
δi is a weighting coefficient determined for each nutrient group;
Gi is the group score for nutrient group i; and
Gi may represent any of: Basic Components, Vitamins, Minerals, Saturated Fats, Monounsaturated Fats, Polyunsaturated Fats, Amino Acids, Other Components, and Exchanges.

14. The computer implemented AI-enabled data refinery system comprising at least one processor and a non-transitory computer-readable memory storing instructions that, when executed by the at least one processor, cause the data refinery system to perform AI-based analyzation, scoring, and reporting of consumables according to claim 1, wherein said analyzation and modeling application is further configured to compute a Relevant Nutrition Score (RNS) utilizing the following equation:
$$R N S = {\sum }_{i = 1}^{N} ( {δ}_{i} \times {N}_{i} )$$
where:
N is the total number of nutritional variables considered;
δi is a nutrient coefficient calculated using AI-based comparison of the Complete User Profile and Food Profile parameters, wherein the comparison evaluates target nutrient amounts and ranges against actual food composition values to derive weighted coefficients; and
Ni is the value of each nutritional variable.

15. The computer implemented AI-enabled data refinery system comprising at least one processor and a non-transitory computer-readable memory storing instructions that, when executed by the at least one processor, cause the data refinery system to perform (AI)-based analyzation, scoring, and reporting of consumables according to claim 1, wherein Relevant Risk Scores (RRS) are generated to assess product-specific health-related risks and impacts associated with food and consumable products, and enable individuals, commercial entities, medical providers, and governmental agencies to address issues related to packaging, ingredient sourcing, storage, preparation practices, additives, preservatives, and other contaminants in order to mitigate health risk exposure for targeted populations and uses.

16. A computer implemented AI-enabled method for calculating a Relevant Nutrition Score (RNS) comprising the steps of:
a) engaging an AI-enabled use and user profiling application configured to collect and calculate user impacts and relevancy based upon disclosed, defined and extrapolated physiological, demographic, health and other data, and determining a use and user-specific profile;
b) engaging an AI-enabled food and consumable profiling module configured for specified products and uses, by compiling all available information, and imputes and adds missing data through analytic inference, and determining a food and consumable use and impact profile;
c) engaging an AI-enabled RNS calculation subsystem and modeling engine for the identified consumable and intended uses by combining the user profile, Relevant Risk Score (RRS), and designated food and consumable uses profiles, calculating an RNS in real-time; and
d) recalculating the RNS in response to dynamically updated user information, foods, pharmaceuticals, and consumables data, uses, treatments, and research.

17. The computer implemented AI-enabled method for calculating a Relevant Nutrition Score (RNS) according to claim 16, wherein Relevant Risk Scores (RRS) are generated to assess product-specific health-related risks and impacts associated with food and consumable products, and enable individuals, commercial entities, medical providers, and governmental agencies to address issues related to packaging, ingredient sourcing, storage, preparation practices, additives, preservatives, and other contaminants in order to mitigate health risk exposure for targeted populations and uses, and user-customized weighting coefficients related to risk.

18. The computer implemented AI-enabled method for calculating a Relevant Nutrition Score (RNS) according to claim 16, wherein said AI-enabled food and consumable profiling module generates a food profile, comprising a set of scores for consumables and health, which dynamically accommodate additional nutritional parameters, user variables, and intended uses, as system data evolves and applies user-aligned vantage points and use-dependent weighting coefficients to each score.

19. The computer implemented AI-enabled method for calculating a Relevant Nutrition Score (RNS) according to claim 16, wherein recalculating the RNS initiates whenever additional use and user inputs, health indicators, and food and consumable composition data and research are made available, ensuring the persistent relevance and personalization of the nutrition score over time to the intended user and defined uses.

20. The computer implemented AI-enabled method for calculating a Relevant Nutrition Score (RNS) according to claim 16, wherein an AI-enabled food and consumable profiling module imputes and adds missing data by applying deductive reasoning, statistical modeling, and domain-aligned algorithms using related product profiles, ingredient information, health impact studies, ingredient research, updated data and research, recognized scientific and regulatory standards, to produce a comprehensive food profile.

21. The computer implemented AI-enabled method for calculating a Relevant Nutrition Score (RNS) according to claim 16, further wherein nutrient categories are used for food and consumable profiling and calculations including two or more of: basic components, vitamins, minerals, saturated fats, monounsaturated fats, polyunsaturated fats, amino acids, ingredient impacts, published side-effects and interactions, lab testing and case studies, health and pharmaceutical research, other nutritional components, and exchanges.

22. The computer implemented AI-enabled method for calculating a Relevant Nutrition Score (RNS) according to claim 16, wherein the RNS is calculated by deriving intended end-user-aligned and use-weighted nutritional variable values according to the following equation:
$$R N S = {\sum }_{i = 1}^{N} ( {δ}_{i} \times {N}_{i} )$$
where δi is the user-customized weighting coefficient; and Ni is the value of the nutritional variable for each category, as determined by the combined provided AI-enabled user profile and provided AI-enabled food and consumable profile.
